(19) United States
(12) Patent Application Publication
(10) Pub. No.: US 2020/0098457 A1
(43) Pub. Date: Mar. 26, 2020

(54) SYSTEM AND METHOD FOR VIAL AMOUNT SELECTION AND DOSAGE VERIFICATION FOR MODULARLY ASSEMBLED MEDICAMENTS

(71) Applicant: Mikael NAYGAUZ, Forrest Hills, NY (US)

(72) Inventor: Mikael NAYGAUZ, Forrest Hills, NY (US)

(21) Appl. No.: 16/298,760

(22) Filed: Mar. 11, 2019

Related U.S. Application Data
(60) Provisional application No. 62/735,023, filed on Sep. 22, 2018.
(60) Provisional application No. 62/744,341, filed on Oct. 11, 2018.
(60) Provisional application No. 62/747,624, filed on Oct. 18, 2018.
(60) Provisional application No. 62/787,340, filed on Jan. 1, 2019.
(60) Provisional application No. 62/797,945, filed on Jan. 29, 2019.

(51) Int. Cl. G16H 20/10; G16H 10/60; G06K 7/14

(52) U.S. Cl. CPC G16H 20/10; G06K 7/1413; G16H 10/60

(57) ABSTRACT

Systems and methods for selecting vial amounts for a modular format medicament assembly and verifying contents of a medicament containers for a modular format medicament assembly including using scanning data-containing tags and unique serial numbers in order to verify that the medicaments match information entered in a software system to provide a quality control of medicament information administered to patients.

### CROSS REFERENCE TO RELATED APPLICATIONS

The present application claims priority to U.S. Provisional Patent Application Ser. No. 62/735,023 filed Sep. 22, 2018; U.S. Provisional Patent Application Ser. No. 62/744,341, entitled SOFTWARE FOR DOSING ASSEMBLY and filed on Oct. 11, 2018; U.S. Provisional Patent Application Ser. No. 62/747,624, entitled SOFTWARE FOR DOSAGE VERIFICATION OF A MODULAR DOSING ASSEMBLY OF MEDICAL SUBSTANCES and filed on Oct. 18, 2018; U.S. Provisional Patent Application Ser. No. 62/787,340, entitled SYSTEM AND METHOD FOR VIAL AMOUNT SELECTION AND DOSAGE VERIFICATION FOR MODULARLY ASSEMBLED MEDICAMENTS and filed on Jan. 1, 2019; and U.S. Provisional Patent Application Ser. No. 62/797,945 filed Jan. 29, 2019 entitled SYSTEM AND METHOD FOR VIAL AMOUNT SELECTION AND DOSAGE VERIFICATION FOR MODULARLY ASSEMBLED MEDICAMENTS, the entire contents of which are expressly incorporated herein by reference.

All of the following United States Patents Applications are hereby incorporated herein by reference in their entireties: Ser. Nos. 16/102,635; 16/102,651; 16/102,657; 16/102,666; 16/102,681; 16/102,698; 16/102,704; 16/102,713; 16/102,719; and 16/233,923.

### FIELD OF THE INVENTION

The embodiments described herein relate generally to pharmacy and nursing software and systems and more specifically to systems, methods, and modes for selecting and verifying that prescribed medicaments that are entered into a patient profile are selected and/or verified prior to administering to that particular patient.

### BACKGROUND OF THE INVENTION

There are many inefficiencies and safety concerns pertaining to processes that involve the preparation and administration of customized amounts of injectable medicaments to patients.

Typically, customized amounts of injectable medicaments are prepared by a pharmacist or other healthcare provider in a pharmacy setting. The pharmacy setting typically has a cleanroom, intravenous (IV) hoods, barrier isolators and other equipment which provides a sterile environment under which the healthcare provider may intermix sterile injectable medicaments. The customized amounts of the sterile injectable medicament are typically measured visually and intermixed by hand and a final dosage amount is provided to a nurse or patient in a bag, a bottle, or a syringe with a pharmacy generated label on the bag, bottle, or syringe that contains the patient's name, drug name, final dosage amount, expiration date, and other information. The process of measuring and intermixing the injectable medicament is prone to human error as the healthcare provider may measure an inaccurate amount of the medicament and/or may mistakenly intermix a wrong medicament. Additionally, when devices such as intravenous pumps are used to intermix injectable pharmaceuticals, errors may still occur in the final dispensing of the product. The bag, bottle, or syringe containing the final dosage amount of the injectable medicament may be mislabeled by a member of the pharmacy staff (a wrong label may be affixed to the incorrect bag, bottle, or syringe). Still additionally, injectable medicaments, particularly injectable chemotherapeutic substances, may be toxic, carcinogenic and/or teratogenic thus an error on the part of the pharmacy staff or healthcare provider involved in the preparation of an injectable medicament may result in disastrous consequences.

Current software used in institutional settings, such as in hospitals, is adapted for checking standard amounts of a medicament to a patient. A standard amount of an injectable medicament is provided in one commercially packaged container. Upon administering the standard amount of the medicament to a patient, the nurse typically scans, using a scanner, the barcode on the single container or scans the barcode on a pharmacy generated label that has been affixed to the container. The pharmacy-nursing software then verifies/checks if the medicament name and the amount of the medicament in the container is the same (matches) the medicament and the amount of the medicament on the patient specific profile and/or on the patient's (patient specific) medicament administration record (MAR). If the software verifies that the medicament and the amount of the medicament provided to the nurse/end-user is the same as the medicament and the amount of the medicament on the patient's profile and/or MAR then the software proceeds to alert the nurse that it is safe to proceed to administer the medicament to the patient. The software may also provide an electronic record on the patient's MAR which stores the medicament name, medicament amount, time medicament administered to patient, and nurse's initials.

Existing systems include U.S. Pat. Nos. 9,449,296; 8,386,070; 8,231,749; 8,212,677; 8,195,328; 8,165,929; 8,111,159; 8,099,339; 8,032,397; 7,933,780; 7,905,832; 7,860,583; 7,835,927; 7,783,383; 7,706,915; 7,645,258; 7,610,115; 7,343,224; 7,317,967; 7,175,081; 7,155,306; 7,107,106; 7,072,840; 7,006,214; 6,935,560; 6,790,198; 6,421,650; 6,202,923; 6,112,182; 607076; 6,021,392; 6,014,631; 5,845,255; 5,781,442; 5,511,594; 5,431,201; 5,317,506; US20170262614A1; US20140142971A1; US20090043253A1; US20070186923A1; US20060265247A1; US20050171815A1; US20040172283A1; US20040162835A1; US20040088187A1; US20040087546A1; US20030140928A1; US20020099334A1; US20010049608A1; US20010001144A1; and WO2012122347A1.

There is currently no method, however, of verifying if a correct medicament or an accurate amount of a medicament is prepared and administered to a patient when the final dosage amount of the medicament is a customized dosage amount since customized dosage amounts of injectable medicaments are typically prepared by pharmacy staff and the commercially provided vials have been discarded after intermixing of the final dosage amount. There is thus a long felt and unmet need for software systems and methods that provide for the verification of customized amounts of injectable medicaments prior to administering the customized amount of the medicament to a patient. There is a need for reliable, user friendly and cost-effective solutions for allowing final product and final dosage amount verification/double-checks.

Additionally, when provided with a modularly assembled final amount of an injectable beneficial substance, there is no method of double-checking and/or verifying if the correct amount of the beneficial substance is to be administered to a patient. Commercially available containers that contain a beneficial substance, such as a medicament, typically have barcodes with the same information embedded in the barcodes such as the identity of the medicament in the container, such as drug name, and the amount of the medicament contained in the container. There is no method of preventing the accidental double-scanning, or repeat scanning, of the same container which introduces safety issues.

Still additionally, when entering customized amounts of beneficial substances the prescriber entering an electronic prescription typically enters an exact customized dose. This inconveniences the pharmacy department and healthcare personnel that intermix the custom amount of the beneficial substance as the amount ordered/entered by the prescriber is usually different than standard commercially packaged/prepackaged amounts. Therefore, after intermixing the customized amount of the beneficial substance, or medicament, there is usually wastage of extra amounts of the medicament. This may be a burden to hospitals and pharmacy departments as chemotherapeutics which are usually given in customized amounts tend to be expensive. Therefore, there is a need in the art for methods to reduce waste of chemotherapeutics and other beneficial substances. Additionally, there is no current system/method in place for verifying at the point-of-care what the pharmacist/pharmacy department has intermixed/prepared since the commercially packaged/prepackaged containers containing the medicament have been discarded by the pharmacist/pharmacy staff and the label affixed to the container housing the customized final dosage supplied to the patient is a pharmacy generated label. Therefore, there is potential for error during the preparation of the custom dosage amount (accidentally using the wrong medicament and/or measuring the wrong amount during preparation) and there is additional potential for error in accidentally mislabeling the container containing the final custom dosage amount with the wrong pharmacy generated label.

Thus, there are certain problems with conventional systems and means for preparing, administering, and double checking (verifying) customized amounts of medicaments to patients. Accordingly, it would be desirable to provide systems, methods, and modes for selecting and/or verifying that prescribed customized amounts of medicaments are accurately selected and dispensed to patients and also verified prior to administration to patients.

### SUMMARY OF THE INVENTION

An object of the embodiments is to substantially solve at least the problems and/or disadvantages discussed above, and to provide at least one or more of the advantages described below.

It is therefore a general aspect of the embodiments to provide systems, methods, and modes for verifying that prescribed customized medicaments that are entered into a profile, entered into a patient specific profile, entered into a medication list, entered into a patient specific medication list and/or displayed/stored in a medication administration record (MAR) for a particular patient are verified/double-checked prior to administering to that particular patient that will obviate or minimize problems of the type previously described.

It is therefore a general aspects of the embodiments to provide systems, methods, and modes for verifying customized final dosages of beneficial substances prepared using commercially packaged/pre-packaged containers in a modular assembly medicament format.

It is therefore a general aspects of the embodiments to provide systems, methods and modes for storing/keeping a record of customized final dosages of beneficial substances that were prepared using commercially packaged/pre-packaged containers in a modular medicament assembly format.

It is also a general aspect of the embodiments to provide systems, methods, and modes for verifying that prescribed customized medicaments intended to be assembled in a modular format that are entered into a profile, entered into a patient specific profile, entered into a medication list, entered into a patient specific medication list and/or displayed/stored in a medication administration record (MAR) are verified/double-checked prior to administering to that particular patient that will obviate or minimize problems of the type previously described.

It is also a general aspect of the embodiments to provide systems, methods, and modes for determining and/or selecting the appropriate amounts and/or quantities of containers containing a beneficial substance that will supply a customized and/or individualized amount of a beneficial substance to a patient.

It is also a general aspect of the embodiments to provide systems, methods, and modes for determining and/or selecting the appropriate amounts and/or quantities of containers containing a medicament that will supply a customized and/or individualized and/or nonstandard amount of the medicament for a modular assembly of the medicament.

It is also a general aspect of the embodiments to provide systems, methods, modes, containers, and/or labels for providing, identifying, comparing, and/or double checking/cross-checking unique identifiers that distinguish containers and/or beneficial substances and/or medicaments from each other.

It is also a general aspect of the embodiments to provide systems, methods, modes, software, and/or computer implemented systems/methods that double-check unique identifiers, scan unique identifiers, cross-check unique identifiers, and/or prevent the duplicate scanning of the same container by a scanner and/or other acquisition device and/or alerting a user if a duplicate scan has occurred or not.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Further features and advantages of the aspects of the embodiments, as well as the structure and operation of the various embodiments, are described in detail below with reference to the accompanying drawings. It is noted that the aspects of the embodiments are not limited to the specific embodiments described herein. Such embodiments are presented herein for illustrative purposes only. Additional embodiments will be apparent to persons skilled in the relevant art(s) based on the teachings contained herein.

According to aspects of the embodiments, a system is provided that provides for the verification of a dosage amount of a beneficial substance provided in a modular assembly format. According to aspects of the embodiments, a system is provided that provides for the verification of a final dosage amount of a beneficial substance provided in a modular assembly format. According to aspects of the embodiments, software and/or computer is provided that provides a double-check/verification of medicament identity and/or medicament final dosage amount provided to a patient. According to aspects of the embodiments, software and/or computer is provided that provides a double-check/verification of medicament identity and/or medicament final customized dosage amount provided to a patient. According to aspects of the embodiments, software and/or computer is provided that provides a double-check/verification of a plurality of medicament identities provided to a patient. According to aspects of the embodiments, software and/or computer is provided that provides a double-check/verification of medicament final dosage amount provided to a patient. According to aspects of the embodiments, software and/or a computer is provided that provides a double-check/verification of a plurality of medicament identities for a final customized dosage amount provided to a patient. According to aspects of the embodiments, software and/or a computer is provided that provides a double-check/verification of a plurality of medicament dosage amounts for a final customized dosage amount provided to a patient. According to aspects of the embodiments, software and/or a computer is provided that provides a double-check/verification of a plurality of medicament dosage amounts packaged in commercially packed containers for a final customized dosage amount provided to a patient. According to aspects of the embodiments, software and/or a computer is provided that provides a double-check/verification of a plurality of medicament dosage amounts packaged in commercially prepacked containers for a final customized dosage amount provided to a patient. According to aspects of the embodiments, software and/or a computer is provided that provides a double-check/verification of a plurality of medicament identities packaged in commercially packed containers for a final customized dosage amount provided to a patient. According to aspects of the embodiments, software and/or a computer is provided that provides a double-check/verification of a plurality of medicament identities packaged in commercially prepacked containers for a final customized dosage amount provided to a patient. According to aspects of the embodiments, software and/or a computer is provided that provides a double-check/verification of a plurality of medicament identities packaged in a plurality of containers. According to aspects of the embodiments, software and/or a computer is provided that provides a double-check/verification of a plurality of medicament identities packaged in a plurality of commercially packaged/prepackaged containers intended for a modular assembly format.

According to aspects of the embodiments, software and/or a computer is provided that provides a double-check/verification of an injectable medicament provided in a modular assembly format. According to aspects of the embodiments, a processor and/or computer is provided to cross-check a plurality of data elements disposed on a plurality of containers housing a beneficial substance against a stored data element. According to aspects of the embodiments, a processor and/or computer is provided to cross-check a plurality of data elements disposed on a plurality of containers housing a beneficial substance against a plurality of stored data elements.

According to aspects of the embodiments, a processor and/or a computer is provided to cross-check a plurality of data elements disposed on a plurality of containers housing a beneficial substance against a patient specific stored data element. According to aspects of the embodiments, a processor and/or a computer is provided to cross-check a plurality of data elements disposed on a plurality of containers housing a beneficial substance against a plurality of stored patient specific data elements. According to aspects of the embodiments, systems and methods are provided for verification of a plurality of data elements disposed on a plurality of containers against patient specific and/or non-patient specific data elements stored in/on software and/or a computer. According to aspects of the embodiments, computers, software, processors, systems, methods and combinations thereof are provided for the prevention of double-scanning (duplicate scanning) of the same container by an end-user.

According to aspects of the embodiments, a processor and/or a computer is provided to add a plurality of data elements and compare the added amount against a patient specific data element. According to aspects of the embodiments, a processor and/or computer is provided to add a plurality of data elements and compare the added amount against a patient specific data element stored and/or displayed on a patient profile. According to aspects of the embodiments, a processor and/or a computer is provided to add a plurality of data elements and compare the added amount against a patient specific data element stored and/or displayed on a medication administration record MAR. According to aspects of the embodiments, a processor and/or a computer is provided to add a plurality of data elements and compare the added amount against a patient specific data element stored and/or displayed and/or linked to a patient medication administration record MAR. According to aspects of the embodiments, a processor and/or a computer is provided to add a plurality of data elements and compare the added amount against a patient specific data element stored and/or displayed and/or linked to a patient specific medication administration record MAR and/or patient medication list and/or a patient specific prescription and/or a prescription.

According to aspects of the embodiments, a processor and/or a computer is provided to add a plurality of data elements and compare the added amount against a patient specific data element stored on a patient specific medication list and/or displayed on a patient specific medication list. According to aspects of the embodiments, a processor and/or computer is provided to add a plurality of data elements and compare the added amount against a patient specific data element stored on a patient specific profile and/or displayed on a patient specific display and/or stored on/in a patient specific prescription and/or linked to a patient specific prescription and/or linked to a prescription. According to aspects of the embodiments, a processor and/or computer is provided to add a plurality of data elements and compare the added amount against a patient specific data element stored on a patient specific medication administration record (MAR). According to aspects of the embodiments, the plurality of data elements are amounts of a beneficial substance. According to aspects of the embodiments, the plurality of the data elements are dose amounts of a beneficial substance. According to aspects of the embodiments, the plurality of the data elements are dosage amounts of a medicament. According to aspects of the embodiments, the plurality of the data elements are dosage amounts of a beneficial substance and/or medicament stored in commercially packaged/prepackaged containers. According to aspects of the embodiments, a processor is provided to cross-check a unique identifier against a list of stored unique identifiers. According to aspects of the embodiments, a processor is provided to cross-check a plurality of unique identifiers against a list of stored unique identifiers.

According to aspects of the embodiments, a processor and/or a computer is provided to cross check a plurality of unique identifiers located on a plurality of containers against a stored list of unique identifiers. According to aspects of the embodiments, the plurality of containers store a beneficial substance/nutritional substance/medicament in the containers. According to aspects of the embodiments, the plurality of containers contain a beneficial substance, a nutritional substance, a medicament and combinations thereof inside the containers. According to aspects of the embodiments, the processor and/or the computer can be adapted for a modular dosing assembly (modular administered medicament) of injectable medical substances. The processor and/or the computer can be adapted to provide a final dosage verification, double-check, and/or electronic verification (double-check) prior to administration of the final amount of the beneficial substance to a patient. According to aspects of the embodiments, after the processor and/or the computer has provided a final dosage verification, it is then safe to proceed to administering the final dosage amount to a patient. According to aspects of the embodiments, after the processor and/or the computer has provided a final dosage verification, it is then safe to proceed to administering the final customized dosage amount to a patient. According to aspects of the embodiments, after the processor and/or the computer has provided a final dosage verification, it is then safe to proceed to administering the final individualized dosage amount to a patient.

According to aspects of the embodiments, a processor and/or computer is provided to add a plurality of data elements and compare the added amount against a patient specific data element. According to aspects of the embodiments, a processor and/or a computer is provided to add a plurality of data elements and compare the added amount against a patient specific data element stored and/or displayed on a patient profile. According to aspects of the embodiments, a processor and/or a computer is provided to add a plurality of data elements and compare the added amount against a patient specific data element stored and/or displayed on a medication administration record MAR. According to aspects of the embodiments, a processor and/or a computer is provided to add a plurality of data elements and compare the added amount against a patient specific data element stored and/or displayed and/or linked to a patient medication administration record MAR linked to a patient specific prescription, linked to a patient specific electronic prescription, and/or linked to a patient specific digital prescription. According to aspects of the embodiments, a processor and/or a computer is provided to add a plurality of data elements and compare the added amount against a patient specific data element stored and/or displayed and/or linked to a patient specific medication administration record MAR and/or patient medication list.

According to aspects of the embodiments, a processor and/or a computer is provided to add a plurality of data elements and compare the added amount against a patient specific data element stored on a patient specific medication list and/or displayed on a patient specific medication list and/or stored on/with a patient specific prescription. According to aspects of the embodiments, a processor and/or a computer is provided to add a plurality of data elements obtained from a modular medicament assembly (modular administered medicament) and compare the added amount against a patient specific data element stored on a patient specific profile and/or displayed on a patient specific profile and/or stored/displayed on a prescription. According to aspects of the embodiments, a processor and/or computer is provided to add a plurality of data elements and compare the added amount against a patient specific data element stored on a patient specific medication administration record (MAR). According to aspects of the embodiments, the plurality of data elements are amounts of a beneficial substance. According to aspects of the embodiments, the plurality of the data elements are dose amounts of a beneficial substance. According to aspects of the embodiments, the plurality of the data elements are dosage amounts of a medicament. According to aspects of the embodiments, the plurality of the data elements are dosage amounts of a beneficial substance and/or medicament stored in commercially packaged/prepackaged containers. According to aspects of the embodiments, a processor and/or a computer is provided to cross-check a unique identifier against a list of stored unique identifiers. According to aspects of the embodiments, a processor is provided to cross-check a plurality of unique identifiers against a list of stored unique identifiers. According to aspects of the embodiments, a computer is provided to cross-check a plurality of unique identifiers against a list of stored unique identifiers. According to aspects of the embodiments, a computer is provided to compare and/or cross-check a plurality of unique identifiers obtained from a modular medicament assembly (modular administered medicament) and to determine if any of the unique identifiers are the same. According to aspects of the embodiments, a computer is provided to compare and/or double-check a plurality of unique identifiers obtained from a plurality of containers used for a modular medicament assembly (MAM) and to determine if a duplicate scan has occurred of the same container.

According to aspects of the embodiments, a processor and/or a computer and/or software is provided to cross check a plurality of unique identifiers located on a plurality of containers against a stored list of unique identifiers. According to aspects of the embodiments, the plurality of containers store a beneficial substance/nutritional substance/medicament in the containers. According to aspects of the embodiments, the plurality of containers contain a beneficial substance, a nutritional substance, a medicament and combinations thereof inside the containers. According to aspects of the embodiments, software can be adapted for a modular dosing assembly of injectable medical substances. The software can be adapted to provide a final dosage verification, double-check, and/or electronic verification (double-check) prior to administration of the final amount of the beneficial substance to a patient. According to aspects of the embodiments, after the software has provided a final dosage verification, it is then safe to proceed to administering the final dosage amount to a patient. According to aspects of the embodiments, after the software has provided a final dosage verification, it is then safe to proceed to administering the final customized dosage amount to a patient. According to aspects of the embodiments, after the software has provided a final dosage verification, it is then safe to proceed to administering the final individualized dosage amount to a patient.

According to aspects of the embodiments, a system is provided for the verification of beneficial substances, comprising: a plurality of containers having a data element on the containers; and software is adapted to compare the data elements from the plurality of the containers against a stored data element. According to aspects of the embodiments, the plurality of containers house a medicament. According to aspects of the embodiments, the plurality of the containers contain a medicament. According to aspects of the embodiments, the plurality of the containers have a medicament disposed inside the containers. According to aspects of the embodiments, the plurality of the containers house a nutritional substance. According to aspects of the embodiments, the plurality of the containers contain a nutritional substance. According to aspects of the embodiments, the nutritional substance may be a protein. According to aspects of the embodiments, the nutritional substance may be an amino acid. According to aspects of the embodiments, the nutritional substance may be a vitamin. According to aspects of the embodiments, the nutritional substance may be a multivitamin. According to aspects of the embodiments, the nutritional substance may be a mineral. According to aspects of the embodiments, the nutritional substance may be a trace element. According to aspects of the embodiments, the nutritional substance may be a lipid. According to aspects of the embodiments, the nutritional substance may be a fat. According to aspects of the embodiments, the nutritional substance may be any substance used in parenteral nutrition that is prepared for patients. According to aspects of the embodiments, the parenteral nutrition may be a total parenteral nutrition formulation. According to aspects of the embodiments, the parenteral nutrition may be for central line administered parenteral nutrition and/or for a peripheral line administered parenteral nutrition. According to aspects of the embodiments, the data element is a barcode. According to aspects of the embodiments, the barcode is adapted to be scanned by a scanning device. According to aspects of the embodiments, the data element is adapted to be readable by a scanning device. According to aspects of the embodiments, the scanning device is a handheld device. According to aspects of the embodiments, the data element is a numeric code. According to aspects of the embodiments, the data element is an alphabetical code. According to aspects of the embodiments, the data element is a numeric code and an alphabetical code.

According to aspects of the embodiments, the stored data element is non-patient specific. According to aspects of the embodiments, the stored data element is patient specific. According to aspects of the embodiments, the stored data element is linked to a patient specific profile. According to aspects of the embodiments, the patient specific profile comprises a medication list. According to aspects of the embodiments, the patient specific profile comprises a list of beneficial substances that have been prescribed to the patient. According to aspects of the embodiments, the patient specific profile is a medication administration record MAR. According to aspects of the embodiments, the stored data element is linked to a patient identifier. According to aspects of the embodiments, the patient identifier is the patient's name. According to aspects of the embodiments, the patient identifier is a medical record number. According to aspects of the embodiments, the patient identifier is the patient's date of birth. According to aspects of the embodiments, the patient identifier may be the patient's weight and/or height. According to aspects of the embodiments, the patient identifier may be any identifier that identifies and distinguishes the patient from other patients. According to aspects of the embodiments, the stored data element is a final dosage amount. According to aspects of the embodiments, the stored data element is a final dosage amount and linked to a patient specific identifier. According to aspects of the embodiments, the patient specific identifier is selected from the group consisting of a name, a medical record number, a room number, a hospital unit number, date of birth and combinations thereof. According to aspects of the embodiments, the final dosage amount is digitally linked to the patient specific identifier. According to aspects of the embodiments, the scanning device is a hand-held scanning device.

According to aspects of the embodiments, the data element is a series of numbers, letters, characters and combinations thereof displayed on the plurality of the containers and/or embedded/encoded on the plurality of the containers. According to aspects of the embodiments, the plurality of the containers are pre-assembled in a modular assembly format, and the plurality of containers are pre-attached in a modular assembly format. According to aspects of the embodiments, the plurality of the containers are not pre-assembled in a modular assembly format, and the plurality of containers are not pre-attached in a modular assembly format. According to aspects of the embodiments, the plurality of the containers are supplied to the end-user, such a nurse, separately and are assembly at the point-of-care.

According to aspects of the embodiments, the plurality of the containers are commercially packaged containers. According to aspects of the embodiments, the plurality of the containers are commercially prepackaged containers. According to aspects of the embodiments, the commercially packaged containers and/or commercially prepackaged containers are supplied to a pharmacy or end user by a drug manufacturer and/or drug distributor. According to aspects of the embodiments, the stored data element is a prescription. According to aspects of the embodiments, the stored data element is an electronic prescription. According to aspects of the embodiments, the data element is patient specific.

According to aspects of the embodiments, the stored data element is linked to a patient specific identifier. According to aspects of the embodiments, the patient specific identifier is a patient's name, a patient's medical record number, a patient's identification code, a patient's date of birth and combinations thereof. According to aspects of the embodiments, the stored data element is electronically and/or digitally linked to a patient specific identifier. According to aspects of the embodiments, the stored data element is electronically and/or digitally linked to a patient specific profile, a patient specific medication list, and/or a patient specific medication administration record.

According to aspects of the embodiments, the stored data element comprises at least one of a medicament amount and a medicament name. According to aspects of the embodiments, the stored data element is a plurality of medicament amounts. According to aspects of the embodiments, the stored data element is one medical amount. According to aspects of the embodiments, the stored data elements are a plurality of medicaments. According to aspects of the embodiments, the stored data elements are a plurality of amounts of the same medicament or the same beneficial substance. According to aspects of the embodiments, the stored data elements are a plurality of amounts of different medicaments or different beneficial substances. According to aspects of the embodiments, the stored data elements are a plurality of amounts of the same medicaments and of different medicaments. According to aspects of the embodiments, the stored data elements are a plurality of amounts of the same beneficial substance. According to aspects of the embodiments, the stored data elements are a plurality of amounts of different beneficial substances. According to aspects of the embodiments, the stored data elements are a plurality of amounts of the same beneficial substance and of different beneficial substances. According to aspects of the embodiments, the stored data element is a plurality of medicament amounts. According to aspects of the embodiments, the stored data element is stored in a computer, and in further aspects of the embodiments, the stored data element is stored in the computer's memory. According to aspects of the embodiments, the stored data element is stored in the cloud, and in further embodiments, the stored data element is stored on a server. According to aspects of the embodiments, the stored data element is stored on a mobile and/or portable device, and in further embodiments the mobile and/or portable device is a handheld device, and in still further embodiments, the handheld device is a phone. According to aspects of the embodiments, the stored data element is stored in an app (application) on a mobile device, and in some embodiments, the mobile device is a phone. According to aspects of the embodiments, the phone is a smartphone.

According to aspects of the embodiments, the medicament amount is a dosage amount. According to aspects of the embodiments, the plurality of the containers contain a beneficial substance, and in embodiments, the plurality of the containers house a beneficial substance. According to aspects of the embodiments, the beneficial substance is a medicament. According to aspects of the embodiments, the plurality of the containers houses the same beneficial substance, and in some embodiments, the plurality of the containers house different beneficial substances. According to aspects of the embodiments, the plurality of the containers house the same medicament, and in some embodiments, the plurality of the containers house different medicaments.

According to aspects of the embodiments, the data elements are adapted to be scanned by an acquisition device, and in some embodiments, the acquisition device is a scanning device. According to aspects of the embodiments, the scanning device is a handheld scanning device. In some embodiments, the containers are vials, and in some embodiments, the containers are syringes. According to aspects of the embodiments, the software is adapted to perform a mathematical calculation of addition, and in some embodiments, the software is adapted to add the data elements on the plurality of the containers. According to aspects of the embodiments, the software is further adapted to add a plurality of the data elements on the plurality of the containers.

According to aspects of the embodiments, the software is adapted to compare the added amount against the stored data element. According to aspects of the embodiments, the stored data element is patient specific, and in some embodiments, the stored data element is linked to a patient profile and/or patient administration record and/or a patient medication list and/or a patient specific prescription. According to aspects of the embodiments, the software is adapted to compare the plurality of the data elements of the containers against a list of stored data elements. According to aspects of the embodiments, the list of stored data elements is patient specific. According to aspects of the embodiments, the stored data element is linked to a patient profile and/or a patient administration record and/or a patient medication list and/or a patient specific prescription and/or a prescription.

According to aspects of the embodiments, the plurality of the containers further comprises a unique identifier, and in some embodiments, the unique identifier is displayed on the plurality of the containers. According to aspects of the embodiments, the unique identifier is embedded in and/or on a data element of the containers. According to aspects of the embodiments, the unique identifier is embedded in a barcode of the containers, and in some embodiments, the unique identifier is encoded in a barcode of the containers. According to aspects of the embodiments, the unique identifier is adapted to prevent repeat (double) scanning of the same vial more than once by a scanning device. According to aspects of the embodiments, the unique identifier is characterized in that it uniquely identifies a container as being different from another container. According to aspects of the embodiments, the unique identifier is characterized in that it differentiates at least two containers containing the same beneficial substance from each other. According to aspects of the embodiments, the unique identifier is characterized in that it differentiates at least two containers containing the same medicament from each other. According to aspects of the embodiments, the unique identifier is characterized in that it differentiates at least two containers containing the same beneficial substance in the same amount from each other. According to aspects of the embodiments, the unique identifier is characterized in that it differentiates at least two containers containing the same medicament in the same amount from each other. According to aspects of the embodiments, the unique identifiers are characterized in that the unique identifiers provide the software, computer, and/or processor with information necessary to prevent duplicate scanning of the same container more than one time.

According to aspects of the embodiments, the software can be used in an institutional setting, and in some embodiments, the institutional setting can be a hospital, a nursing home, a clinic, any combinations thereof, among other institutions. According to aspects of the embodiments, a system is provided for the verification of beneficial substances, comprising: a plurality of containers having a data element on the containers; and software adapted to compare the data elements from the plurality of the containers against a stored patient specific data element. According to aspects of the embodiments, the patient specific data element is a dosage amount intended for a particular patient, and in some embodiments, the patient specific data element is a final dosage amount for a particular patient.

According to aspects of the embodiments, the patient specific data element comprises a plurality of dosage amounts for a particular patient, and in some embodiments, the patient specific data element comprises a list of dosage amounts displayed on a patient profile. According to aspects of the embodiments, the patient profile is a patient's medication list, and in one or more embodiments, the patient profile is a medication administration record (MAR). According to aspects of the embodiments, the patient specific data elements comprises a plurality of patient specific data elements, and in some embodiments, the plurality of patient specific data elements comprises a list of container amounts. According to aspects of the embodiments, the plurality of containers house a medicament, and in some embodiments, the plurality of the containers contain a medicament. According to aspects of the embodiments, the plurality of containers house a nutritional substance, and in some embodiments, the plurality of the containers container a nutritional substance. According to aspects of the embodiments, the nutritional substance is a protein, an amino acid, a lipid, a fat, a vitamin, a multivitamin, a mineral, a trace element, an electrolyte and combinations thereof.

According to aspects of the embodiments, the data element can be a barcode, and in some embodiments, the barcode is adapted to be scanned by a scanning device. According to aspects of the embodiments, the data element is adapted to be readable by a scanning device, and in some embodiments, the scanning device is a handheld device. According to aspects of the embodiments, the stored data element can be non-patient specific, and in some embodiments, the stored data element is patient specific. According to aspects of the embodiments, the stored data element is a final dosage amount, and in some embodiments, the stored data element is a final dosage amount and linked to a patient specific identifier. According to aspects of the embodiments, the patient specific identifier is selected from the group consisting of a name, a medical record number, a room number, a hospital unit number, and a date of birth, and combinations thereof. According to aspects of the embodiments, the final dosage amount is digitally linked to the patient specific identifier. According to aspects of the embodiments, the scanning device is a hand-held scanning device, and in some embodiments, the data element is a series of numbers, letters, characters and combinations thereof displayed on the plurality of the containers.

According to aspects of the embodiments, the plurality of the containers can be pre-assembled in a modular assembly format, and in some embodiments, the plurality of containers are pre-attached in a modular assembly format. According to aspects of the embodiments, the plurality of the containers are not pre-assembled in a modular assembly format, and in some embodiments, the plurality of containers are not pre-attached in a modular assembly format. According to aspects of the embodiments, the plurality of containers can be supplied to the end-user, such as a nurse, as separated containers/devices, such that they can be assembled at the point-of-care.

According to aspects of the embodiments, the plurality of containers can be commercially packaged containers, and in some embodiments, the plurality of containers can be commercially pre-packaged containers. According to aspects of the embodiments, the commercially packaged containers are supplied by drug manufacturers. According to aspects of the embodiments, the commercially packaged containers are manufactured and/or produced by drug manufacturers. According to aspects of the embodiments, the commercially prepackaged containers are supplied to distributors and/or healthcare institutions by pharmaceutical manufacturers. According to aspects of the embodiments, the commercially prepackaged containers are manufactured/produced and/or filled with beneficial substances by pharmaceutical manufacturers. According to aspects of the embodiments, the stored data element is a prescription, and in some embodiments, the stored data element is an electronic prescription. According to aspects of the embodiments, the electronic prescription contains an amount of a beneficial substance. According to aspects of the embodiments, the electronic prescription contains a plurality of dosage amounts of the same beneficial substance. According to aspects of the embodiments, the electronic prescription contains a plurality of dosage amounts of different beneficial substances. According to aspects of the embodiments, the stored data element is patient specific, and in some embodiments, the stored data element is linked to a patient specific identifier. According to aspects of the embodiments, the patient specific identifier is a name, medical record number, an identification code, a date of birth, and combinations thereof. According to aspects of the embodiments, the stored data element is electronically and/or digitally linked to a patient specific identifier. According to aspects of the embodiments, the stored data element is electronically and/or digitally linked to a patient specific profile, patient specific medication list, and/or patient specific medication administration record.

According to aspects of the embodiments, the stored data element comprises at least one of a medicament amount, and a medicament name. According to aspects of the embodiments, the stored data element is a plurality of medicament amounts, and in some embodiments, the stored data element is one medical amount. According to aspects of the embodiments, the stored data element is a plurality of medicament amounts. According to aspects of the embodiments, the stored data element is stored in a computer, and in some embodiments, the stored data element is stored in the computer's memory. According to aspects of the embodiments, the stored data element is stored in the cloud, and in some embodiments, the stored data element is stored on a server. According to aspects of the embodiments, the stored data element is stored on a mobile and/or portable device, and in some embodiments the mobile and/or portable device is a handheld device, and in still further aspects of the embodiments, the handheld device is a phone.

According to aspects of the embodiments, the stored data element is stored in an app (application) on a mobile device, and in some embodiments, the mobile device is a phone, and in still further aspects of the embodiments, the phone is a smartphone. According to aspects of the embodiments, the stored data element is stored on a computer, the computer may be a desktop computer or a laptop computer. According to aspects of the embodiments, the stored data element is stored on or in a server, on or in the cloud, and/or combinations thereof. According to aspects of the embodiments, the medicament amount is a dosage amount. According to aspects of the embodiments, the plurality of the containers contain a beneficial substance, and in some embodiments, the plurality of containers can house a beneficial substance. According to aspects of the embodiments, the beneficial substance is a medicament, and in some embodiments, the plurality of containers house the same beneficial substance. According to aspects of the embodiments, the plurality of the containers house different beneficial substances, and in some embodiments, the plurality of containers house the same medicament, while in still further aspects of the embodiments, the plurality of the containers house different medicaments. According to aspects of the embodiments, the beneficial substance is a nutritional substance. According to aspects of the embodiments, the plurality of the containers house and/or container the same nutritional substance, while in other embodiments the plurality of the containers house and/or contain different nutritional substances. According to aspects of the embodiments, the nutritional substance may be a protein, an amino acid, a lipid, a fat, a vitamin, a multivitamin, a mineral, a trace element and combinations thereof. According to aspects of the embodiments, the beneficial substance may be a diagnostic substance.

According to aspects of the embodiments, the data elements are adapted to be scanned by an acquisition device. According to aspects of the embodiments, the acquisition device is a scanning device, and in some embodiments, the scanning device is a handheld scanning device. According to aspects of the embodiments, the containers are vials, and in some embodiments, the containers are syringes though other containers are contemplated such as cartridges, bottles, etc. According to aspects of the embodiments, the software is adapted to perform a mathematical calculation of addition. According to aspects of the embodiments, the software is adapted to add the data elements on the plurality of the containers. In According to aspects of the embodiments, the software is further adapted to add a plurality of the data elements on the plurality of the containers. According to aspects of the embodiments, the software is adapted to compare the added amount against the stored data element. According to aspects of the embodiments, the stored data element is patient specific, and in some embodiments, the stored data element is linked to a patient profile, a patient medication list, a patient specific prescription, an electronic prescription, a medication administration record and/or a patient administration record.

According to aspects of the embodiments, the software is adapted to compare the plurality of the data elements of the containers against a list of stored data elements. According to aspects of the embodiments, the list of stored data elements is patient specific. According to aspects of the embodiments, the list of stored data elements is linked to a patient profile, a patient's medication list, a patient administration record and/or a patient medication administration record. According to aspects of the embodiments, the list of stored data elements is linked to a patient specific profile, a patient specific medication list, a patient specific patient administration record, and/or a patient specific medication administration record MAR. According to aspects of the embodiments, the plurality of containers further comprises a unique identifier. According to aspects of the embodiments, the unique identifier is displayed on the plurality of the containers. According to aspects of the embodiments, the unique identifier is embedded in and/or on a data element of the containers. According to aspects of the embodiments, the unique identifier is embedded in a barcode of the containers. According to aspects of the embodiments, the unique identifier is encoded in a barcode of the containers. According to aspects of the embodiments, the unique identifier is adapted to prevent repeat (double) scanning of the same vial more than once by a scanning device. According to aspects of the embodiments, a unique identifier is adapted/configured to differentiate one container from another container. According to aspects of the embodiments, the unique identifier is adapted/configured to differentiate each container supplied in a modular assembly format. According to aspects of the embodiments, the unique identifier is adapted/configured to differentiate containers housing the same beneficial substance. According to aspects of the embodiments, the unique identifier is adapted/configured to differentiate containers housing the same medicament and/or nutritional substance. According to aspects of the embodiments, the unique identifier is adapted/configured to differentiate containers housing the same medicament in the same amount. According the aspects of the embodiments, the unique identifier is adapted/configured to differentiate containers housing the same beneficial substance in the same amount. According to the aspects of the embodiments, the beneficial substance may be a nutritional substance such as an amino acid, a protein, a lipid, a vitamin, a multivitamin, a mineral, and/or a trace element. According to aspects of the embodiments, the software can be used in an institutional setting. According to aspects of the embodiments, the institutional setting can be a hospital, a nursing home, a clinic, and combinations thereof. According to aspects of the embodiments, the software can be used at the point of care. According to aspects of the embodiments, the point of care can be a patient's home.

According to aspects of the embodiments, a system is provided for the verification of an amount of a medicament, comprising: a plurality of containers housing a medicament, the containers having a data element disposed on the containers, the data element characterized in that it represents about an amount of the medicament stored in the containers; and software that is adapted to compare the data elements from the plurality of containers against a stored data element. According to further aspects of the embodiments, a system is provided for the verification of an amount of a medicament, comprising: a plurality of containers housing a medicament, the containers having a data element disposed on the containers, the data element characterized in that it represents about an amount of the medicament stored in the containers; and software is adapted to compare the data elements from the plurality of containers against a stored patient specific data element. According to aspects of the embodiments, the patient specific data element is linked to a patient specific profile, a patient specific medication list, a patient specific administration record, a patient specific prescription, a patient specific electronic prescription, a patient specific digital prescription, a patient specific medication administration record MAR, a patient's patient name, a patient's date of birth, a patient's medical record number, a patient's room number, a patient's unit/floor number, a patient assigned bed, a patient specific identifying code and combinations thereof.

According to further aspects of the embodiments, a system is provided for the verification of a modular format medicament assembly, comprising: a modular format medicament assembly having a plurality of containers, wherein the plurality of the containers having a data element disposed on the containers; an acquisition device for acquiring the data elements from the plurality of containers; and a processor and/or a computer that is adapted to compare the data elements from the plurality of the containers against a stored data element. According to aspects of the embodiments, the acquisition device is a scanning device, and in some embodiments, the scanning device is handheld. According to aspects of the embodiments, the processor and/or the computer is further adapted to receive a plurality of data elements from the plurality of the containers. According to aspects of the embodiments, the stored data element it a patient specific data element. According to aspects of the embodiments, the processor and/or the computer is further adapted to receive a plurality of data elements from a memory element. According to aspects of the embodiments, the memory element may be a stored data element. According to aspects of the embodiments, the memory element may be a patient specific memory element. According to aspects of the embodiments, the modular format medicament assembly is characterized in that a plurality of containers each housing/containing a beneficial substance are attached to a bag/bottle/syringe having a plurality of ports, the ports configured to engage/couple with the plurality of the containers. According to aspects of the embodiments, the modular format medicament assembly is characterized in that a plurality of containers housing/containing a beneficial substance are configured to engage/couple with each other and to establish a fluidic communication with each other.

According to aspects of the embodiments, the processor and/or computer is further adapted to receive a data element from a patient specific profile, a patient specific medication list, a patient specific prescription, a patient specific medication administration record, and combinations thereof. According to aspects of the embodiments, the patient specific profile is a patient's medicament profile. According to aspects of the embodiments, the patient specific profile is a patient's medication list. According to aspects of the embodiments, the patient specific profile is a patient's medication administration record. According to aspects of the embodiments, the processor and/or the computer is further adapted to receive a plurality of data elements from the plurality of the containers and adapted to receive a plurality of data elements from a memory element. According to aspects of the embodiments, the processor and/or the computer is further adapted to receive a plurality of data elements from the plurality of the containers and adapted to receive a plurality of data elements from a patient specific memory element. According to aspects of the embodiments, the processor and/or the computer is further adapted to receive a plurality of data elements from the plurality of the containers and adapted to receive a plurality of data elements from a patient specific profile. According to aspects of the embodiments, the patient specific profile is a patient specific medication list, a patient specific administration record, a patient specific medication administration record, and combinations thereof. According to aspects of the embodiments, the processor and/or the computer is further adapted to receive a plurality of data element from the plurality of the containers and adapted to receive a plurality of data elements from a patient specific prescription.

According to aspects of the embodiments, the processor and/or the computer is further adapted to receive a plurality of data elements from the plurality of the containers and adapted to receive a plurality of data elements from a patient specific profile and/or a patient specific electronic prescription. According to aspects of the embodiments, the processor and/or the computer is further adapted to receive a plurality of data elements from the plurality of the containers and adapted to receive a plurality of data elements from a patient specific medication profile. According to aspects of the embodiments, the processor and/or the computer is further adapted to receive a plurality of data elements from the plurality of the containers and adapted to receive a plurality of data elements from a patient specific medication list. According to aspects of the embodiments, the processor and/or the computer is further adapted to receive a plurality of data elements from the plurality of the containers and adapted to receive a plurality of data elements from a patient specific medication administration record (MAR). According to aspects of the embodiments, the processor can be located on a server, inside the computer, inside portable device, in the cloud, and combinations thereof. According to aspects of the embodiments, the computer may be a desktop computer, a laptop computer, a handheld device such as a tablet, a smartphone, etc.

According to aspects of the embodiments, comparing the data elements from the plurality of the containers against a stored data element comprises cross-checking the data elements from the plurality of the containers against the stored data element. According to aspects of the embodiments, the stored data element is one data element. According to aspects of the embodiments, the stored data elements can comprise a plurality of data elements. According to aspects of the embodiments, cross-checking is double-checking. According to aspects of the embodiments, double-checking is comparing if at least one data element of the containers is the same as at least one stored data element. According to aspects of the embodiments, double-checking is comparing if a plurality of data elements from the plurality of the containers is the same as the plurality of the stored data elements. According to aspects of the embodiments, double-checking is comparing if at least one data element of the containers is different than at least one stored data element. According to aspects of the embodiments, double-checking is comparing if a plurality of data elements from the plurality of the containers is different than the plurality of the stored data elements. According to aspects of the embodiments, the comparing is determining. According to aspects of the embodiments, the plurality of the containers house a medicament. According to aspects of the embodiments, the plurality of the containers contain a medicament. According to aspects of the embodiments, the data element is a barcode. According to aspects of the embodiments, the barcode is adapted to be scanned by a scanning device. According to aspects of the embodiments, the data element is adapted to be readable by a scanning device. According to aspects of the embodiments, the scanning device is handheld.

According to aspects of the embodiments, the stored data element is non-patient specific. According to aspects of the embodiments, the stored data element is patient specific. According to aspects of the embodiments, the stored data element is a final dosage amount. According to aspects of the embodiments, the stored data element is a final dosage amount and linked to a patient specific identifier. According to aspects of the embodiments, the patient specific identifier is selected form the group a name, a medical record number, a room number, a hospital unit number, date of birth, and combinations thereof. According to aspects of the embodiments the final dosage amount is digitally linked to the patient specific identifier. According to aspects of the embodiments, the scanning device is a hand-held scanning device. According to aspects of the embodiments, the data element is a series of number, letters, characters and combinations thereof displayed on the plurality of the containers. According to aspects of the embodiments, the containers are vials, bottles, cartridges, syringes, and combinations thereof.

According to aspects of the embodiments, the plurality of the containers are pre-assembled in a modular assembly format. According to aspects of the embodiments, the plurality of containers are pre-attached in a modular assembly format. According to aspects of the embodiments, the plurality of the containers are not pre-assembled in a modular assembly format. According to aspects of the embodiments, the plurality of containers are not pre-attached in a modular assembly format. According to aspects of the embodiments, the plurality of the containers are supplied to the end-user, such a nurse, separately and are assembled at the point-of-care by the patient, nurse, or other healthcare provider. According to aspects of the embodiments, the plurality of the containers are commercially packaged containers. According to aspects of the embodiments, the plurality of the containers are commercially prepackaged containers. According to aspects of the embodiments, the stored data element is a prescription. According to aspects of the embodiments, the stored data element is an electronic and/or a digital prescription. According to aspects of the embodiments, the stored data element is patient specific. According to aspects of the embodiments, the prescription is written by a healthcare provider. According to aspects of the embodiments, the electronic prescription is entered in a computer and/or software by a doctor, nurse practitioner, physician assistant, pharmacist, or other healthcare provider.

According to aspects of the embodiments, the stored data element is linked to a patient specific identifier. According to aspects of the embodiments, the identifier is a name, medical record number, an identification code, a date of birth and combinations thereof. According to aspects of the embodiments, the stored data element is electronically and/or digitally linked to a patient specific identifier. According to aspects of the embodiments, the stored data element is electronically and/or digitally linked to a patient specific profile and/or medication administration record. According to aspects of the embodiments, the stored data element comprises at least one of a medicament amount and a medicament name. According to aspects of the embodiments, the stored data element is a plurality of medicament amounts. According to aspects of the embodiments, the stored data element is one medical amount. According to aspects of the embodiments, the stored data element is a plurality of medicament amounts.

According to aspects of the embodiments, the stored data element is stored in a computer. According to aspects of the embodiments, the stored data element is stored in the computer's memory. According to aspects of the embodiments, the stored data element is stored in the cloud. According to aspects of the embodiments, the stored data element is stored on a server. According to aspects of the embodiments, the stored data element is stored on a mobile and/or portable device. According to aspects of the embodiments, the mobile and/or portable device is a handheld device. According to aspects of the embodiments, the handheld device is a phone.

According to aspects of the embodiments, the stored data element is stored in an app (application). According to aspects of the embodiments, the app (application) stores the stored data element on a mobile device. According to aspects of the embodiments, the mobile device is a phone. According to aspects of the embodiments, the phone is a smartphone. According to aspects of the embodiments, the app (application) stores the stored data element in the cloud. According to aspects of the embodiments, the app (application) stores the stored data element on a server. According to aspects of the embodiments, the app (application) stores a plurality of stored data elements. According to aspects of the embodiments, the stored data element is a medicament amount. According to aspects of the embodiments, the stored data element is a dosage amount. According to aspects of the embodiments, the plurality of the containers contain a beneficial substance. According to aspects of the embodiments, the plurality of the containers house a beneficial substance. According to aspects of the embodiments, the beneficial substance is a medicament. According to aspects of the embodiments, the beneficial substance is a nutritional substance such as an amino acid, a protein, a lipid, a fat, a multivitamin, a vitamin, a mineral, a trace element and combinations thereof. According to aspects of the embodiments, the beneficial substance is a diagnostic substance. According to aspects of the embodiments, the plurality of the containers houses the same beneficial substance. According to aspects of the embodiments, the plurality of the containers house different beneficial substances. According to aspects of the embodiments, the plurality of the containers house the same medicament. According to aspects of the embodiments, the plurality of the containers house different medicaments.

According to aspects of the embodiments, the data elements are adapted to be scanned by an acquisition device. According to aspects of the embodiments, the acquisition device is a scanning device. According to aspects of the embodiments, the scanning device is a handheld scanning device. According to aspects of the embodiments, the containers are vials, bottles, containers with plungers, and/or cartridges. According to aspects of the embodiments, the containers are syringes. According to aspects of the embodiments, the software is adapted to perform a mathematical calculation of addition. According to aspects of the embodiments, the software is adapted to add the data elements on the plurality of the containers. According to aspects of the embodiments, the software is further adapted to add a plurality of the data elements on the plurality of the containers.

According to aspects of the embodiments, the software is adapted to compare the added amount against the stored data element. According to aspects of the embodiments, the stored data element is patient specific. According to aspects of the embodiments, the stored data element is linked to a patient profile, a patient specific medication list, a patient administration record, and/or a patient medication administration record. According to aspects of the embodiments, the software is adapted to compare the plurality of the data elements of the plurality of the containers against a list of stored data elements. According to aspects of the embodiments, the list of stored data elements is patient specific. According to aspects of the embodiments, the stored data element is linked to a patient profile, a patient medication list, a patient administration record, and/or a patient medication administration record.

According to aspects of the embodiments, the plurality of the containers further comprises a unique identifier. According to aspects of the embodiments, the unique identifier is displayed on the plurality of the containers. According to aspects of the embodiments, the unique identifier is embedded in and/or on a data element of the containers. According to aspects of the embodiments, the unique identifier is embedded in a barcode of the containers. According to aspects of the embodiments, the unique identifier is encoded in a barcode of the containers. According to aspects of the embodiments, the unique identifier is adapted to prevent repeat (double) scanning of the same vial more than once by a scanning device. According to aspects of the embodiments, the software is used in an institutional setting. According to aspects of the embodiments, the institutional setting can be a hospital, a nursing home, a clinic and combinations thereof. According to aspects of the embodiments, the software is used at the point of care. According to aspects of the embodiments, the point of care is a patient's home. According to aspects of the embodiments, the point of care is a remote location where healthcare is not readily available such as a battlefield, third world countries, etc.

According to aspects of the embodiments, a system is provided for the verification of a modular format medicament assembly, comprising: a modular format medicament assembly having a plurality of containers, wherein the plurality of the containers having a data element disposed on the containers; an acquisition device for acquiring the data elements from the plurality of containers; and a processor and/or a computer adapted to compare the data elements from the plurality of the containers against a stored patient specific data element. According to aspects of the embodiments, the modular format medicament assembly comprises a first container having a plurality of ports configured to accept plurality of containers containing a medicament and/or a beneficial substance. According to aspects of the embodiments, the first container is a bag or a bottle. According to aspects of the embodiments, the first container is a syringe and/or a barrel with a plunger. According to aspects of the embodiments, the modular format medicament assembly comprises a plurality of containers configured to engage/couple with each other and form a fluidic communication with each other wherein a beneficial substance, such as a medicament/nutritional substance, flows through the containers and into an infusion line which delivers the beneficial substance to a patient. According to aspects of the embodiments, the modular format medicament assembly is configured to provide a customized amount of a beneficial substance to a patient/end-user without manually measuring and/or mixing/intermixing the contents of the containers. According to aspects of the embodiments, the modular format medicament assembly is characterized in that it provides an individualized amount of a beneficial substance to a patient/end-user without manual manipulation of the beneficial substance. According to aspects of the embodiments, the modular format medicament assembly is characterized in that it provides a customized amount of a beneficial substance to a patient/end-user without manipulation of the contents of the containers (no withdrawing or injecting the beneficial substance into or out of the containers using syringes. According to aspects of the embodiments, the modular format medicament assembly is characterized in that is provides a customized amount of a beneficial substance to a patient/end-user in a commercially packaged format. According to aspects of the embodiments, the modular format medicament assembly is characterized in that is provides a customized amount of a beneficial substance to a patient/end-user in a commercially prepackaged format. According to aspects of the embodiments, the modular format medicament assembly described/disclosed herein in this paragraph is applicable to all systems and methods disclosed in this invention's specification.

According to aspects of the embodiments, the patient specific data element is linked to a patient identifier. According to aspects of the embodiments, the stored patient specific data element is linked to a patient identifier. According to aspects of the embodiments, a plurality of patient specific data elements are linked to a patient identifier. According to aspects of the embodiments, a plurality of stored patient specific data elements are linked to a patient identifier. According to aspects of the embodiments, a plurality of patient specific data elements are linked to the same patient identifier. According to aspects of the embodiments, a plurality of stored patient specific data elements are linked to the same patient identifier. According to aspects of the embodiments, the patient specific data element is digitally linked and/or electronically linked to a patient identifier. According to aspects of the embodiments, the stored patient specific data element is digitally linked and/or electronically linked to a patient identifier. According to aspects of the embodiments, the link/linking is characterized in that when a computer and/or processor pulls up/accesses a data element a patient corresponding to the pulled up/accessed data element is identified. According to aspects of the embodiments, the link/linking is characterized in that when a computer and/or processor pulls up/accesses a particular patient a corresponding data element for the particular patient, optionally a plurality of corresponding data elements for the particular patient are identified. According to aspects of the embodiments, the patient identifier is selected from a name, an identification code, an identification number, a social security number, a medical record number, date of birth and combinations thereof. According to aspects of the embodiments, the patient identifier can be a room number and/or a bed number. According to aspects of the embodiments, the patient specific data element is linked to a patient profile. According to aspects of the embodiments, the stored patient specific data element is linked to a patient profile. According to aspects of the embodiments, the patient specific data element is linked to a patient specific profile. According to aspects of the embodiments, the stored patient specific data element is linked to a patient specific profile. According to aspects of the embodiments, the patient specific data element is linked to a patient medication profile. According to aspects of the embodiments, the stored patient specific data element is linked to a patient medication profile. According to aspects of the embodiments, the patient specific data element is linked to a patient specific medication profile. According to aspects of the embodiments, the stored patient specific data element is linked to a patient specific medication profile. According to aspects of the embodiments, the patient specific data element is linked to a medication administration record. According to aspects of the embodiments, the stored patient specific data element is linked to a medication administration record. According to aspects of the embodiments, the patient specific data element is linked to a patient specific medication administration record. According to aspects of the embodiments, the stored patient specific data element is linked to a patient specific medication administration record.

According to aspects of the embodiments, a system is provided for the verification of a modular format medicament assembly, comprising: a modular format medicament assembly having a plurality of containers, wherein the plurality of the containers having a data element disposed on the containers; an acquisition device for acquiring the data elements from the plurality of containers; and a processor and/or a computer that is adapted to compare the data elements from the plurality of the containers against a plurality of stored patient specific data elements.

According to aspects of the embodiments, the plurality of the patient specific data elements are linked to a patient identifier. According to aspects of the embodiments, the plurality of the stored patient specific data elements are linked to a patient identifier. According to aspects of the embodiments, the plurality of the patient specific data elements are digitally linked and/or electronically linked to a patient identifier. According to aspects of the embodiments, the plurality of the stored patient specific data elements are digitally linked and/or electronically linked to a patient identifier. According to aspects of the embodiments, the patient identifier is selected from a name, an identification code, an identification number, date of birth and combinations thereof. According to aspects of the embodiments, the patient identifier can be a room number and/or a bed number. According to aspects of the embodiments, linked may mean that when a data element is pulled-up/identified/used by a computer, software, and/or processor a corresponding patient's name, patient's medical record number (MR#), patient's identification code, patient's room number, patient's bed number, patient's date of birth, patient's social security number, and/or other similar patient specific information is pulled-up/identified/used/extracted by the computer, the software, and/or the processor. The vice versa is also true, wherein linked may mean that when a corresponding patient's name, MR#, patient's identification code, patient's room number, patient's bed number, patient's date of birth, patient's social security number, and/or other similar patient specific information is pulled-up/identified used/extracted by the computer, the software, and/or the processor a corresponding data element, optionally a plurality of corresponding data elements, is/are pulled-up/identified/used/extracted by the computer, the software, and/or the processor.

According to aspects of the embodiments, the plurality of patient specific data elements are linked to a patient profile. According to aspects of the embodiments, the plurality of patient specific data elements are linked to a patient specific profile. According to aspects of the embodiments, the plurality of patient specific data elements are linked to a patient medication profile. According to aspects of the embodiments, the plurality of patient specific data elements are linked to a patient specific medication profile. According to aspects of the embodiments, the plurality of patient specific data elements are linked to a medication administration record. According to aspects of the embodiments, the plurality of the stored patient specific data elements are linked to a patient profile and/or a prescription. According to aspects of the embodiments, the plurality of the stored patient specific data elements are linked to a patient specific profile and/or an electronic/digital prescription. According to aspects of the embodiments, the plurality of stored patient specific data elements are linked to a patient medication profile. According to aspects of the embodiments, the plurality of stored patient specific data elements are linked to a patient specific medication profile. According to aspects of the embodiments, the plurality of stored patient specific data elements are linked to a medication administration record.

According to aspects of the embodiments, the plurality of patient specific data elements are linked to a patient specific medication administration record and/or a patient specific prescription. According to aspects of the embodiments, the acquisition device is a scanning device adapted to scan the data element disposed on the containers. According to aspects of the embodiments, the data element disposed on the containers is a barcode, the barcode adapted to be scanned by the acquisition device. According to aspects of the embodiments, the plurality of the containers house a medicament. According to aspects of the embodiments, the plurality of the containers contain a medicament. According to aspects of the embodiments, the plurality of the containers house a nutritional substance. According to aspects of the embodiments, the plurality of the containers contain a nutritional substance. According to aspects of the embodiments, the nutritional substance is an amino acid, a protein, a lipid, a fat, a mineral, a vitamin, a multivitamin, and/or trace element.

According to aspects of the embodiments, the data element is a barcode. According to aspects of the embodiments, the barcode is adapted to be scanned by a scanning device. According to aspects of the embodiments, the data element is adapted to be readable by a scanning device. According to aspects of the embodiments, the scanning device is a handheld. According to aspects of the embodiments, the stored data element is non-patient specific. According to aspects of the embodiments, the stored data element is patient specific.

According to aspects of the embodiments, the stored data element is a final dosage amount. According to aspects of the embodiments, the stored data element is a final dosage amount and linked to a patient specific identifier. According to aspects of the embodiments, the patient specific identifier is selected form the group a name, a medical record number, an identification code, a room number, a hospital unit number, date of birth and combinations thereof. According to aspects of the embodiments the final dosage amount is digitally linked to the patient specific identifier.

According to aspects of the embodiments, the scanning device is a hand-held scanning device. According to aspects of the embodiments, the data element is a series of number, letters, characters and combinations thereof displayed on the plurality of the containers. According to aspects of the embodiments, the plurality of the containers are pre-assembled in a modular assembly format. According to aspects of the embodiments, the plurality of containers are pre-attached in a modular assembly format. According to aspects of the embodiments, the plurality of the containers are not pre-assembled in a modular assembly format. According to aspects of the embodiments, the plurality of containers are not pre-attached in a modular assembly format. According to aspects of the embodiments, the plurality of the containers are supplied to the end-user, such a nurse, separately and are assembled at the point-of-care. According to aspects of the embodiments, the modular assembly format provides a customized and/or individualized amount of a beneficial substance to a patient. According to aspects of the embodiments, the plurality of the containers provide a customized and/or individualized amount of a beneficial substance to a patient. According to aspects of the embodiments, the modular assembly format and/or the plurality of the containers provide a customized amount of a beneficial substance/medicament/nutritional substance to a patient in a commercially packaged/prepackaged format.

According to aspects of the embodiments, the plurality of the containers are commercially packaged containers. According to aspects of the embodiments, the plurality of the containers are commercially prepackaged containers. According to aspects of the embodiments, the stored data element is a prescription. According to aspects of the embodiments, the stored data element is an electronic prescription. According to aspects of the embodiments, the stored data element is patient specific. According to aspects of the embodiments, the stored data element is linked to a patient specific identifier. According to aspects of the embodiments, the identifier is a name, medical record number, an identification code, a date of birth and combinations thereof. According to aspects of the embodiments, the stored data element is electronically and/or digitally linked to a patient specific identifier. According to aspects of the embodiments, the stored data element is electronically and/or digitally linked to a patient specific profile, a patient specific medication list, and/or medication administration record MAR.

According to aspects of the embodiments, the stored data element comprises at least one of a medicament amount and a medicament name. According to aspects of the embodiments, the stored data element is a plurality of medicament amounts. According to aspects of the embodiments, the stored data element is one medical amount. According to aspects of the embodiments, the stored data element is a plurality of medicament amounts.

According to aspects of the embodiments, the stored data element is stored in a computer. According to aspects of the embodiments, the stored data element is stored in the computer's memory. According to aspects of the embodiments, the stored data element is stored in the cloud. According to aspects of the embodiments, the stored data element is stored on a server. According to aspects of the embodiments, the stored data element is stored on a mobile and/or a portable device. According to aspects of the embodiments the mobile and/or portable device is a handheld device. According to aspects of the embodiments, the handheld device is a phone. According to aspects of the embodiments, the stored data element is stored in an app (application) on a mobile device. According to aspects of the embodiments, the mobile device is a phone. According to aspects of the embodiments, the phone is a smartphone. According to aspects of the embodiments, the stored data element is stored in an app's (application's) server. According to aspects of the embodiments, the stored data element is stored in the cloud under an app (application) specific server and/or memory storage element. According to aspects of the embodiments, the medicament amount is a dosage amount.

According to aspects of the embodiments, the plurality of the containers contain a beneficial substance. According to aspects of the embodiments, the plurality of the containers house a beneficial substance. According to aspects of the embodiments, the beneficial substance is a medicament. According to aspects of the embodiments, the beneficial substance is a nutritional substance and/or a diagnostic substance. According to aspects of the embodiments, the plurality of the containers houses the same beneficial substance. According to aspects of the embodiments, the plurality of the containers house different beneficial substances. According to aspects of the embodiments, the plurality of the containers house the same medicament. According to aspects of the embodiments, the plurality of the containers house different medicaments.

According to aspects of the embodiments, the data elements are adapted to be scanned by an acquisition device. According to aspects of the embodiments, the acquisition device is a scanning device. According to aspects of the embodiments, the scanning device is a handheld scanning device. According to aspects of the embodiments, the containers are vials. According to aspects of the embodiments, the containers are syringes. According to aspects of the embodiments, the containers are bottles. According to aspects of the embodiments, the containers have a plunger. According to aspects of the embodiments, the containers have protective side walls.

According to aspects of the embodiments, the software and/or computer is adapted to perform a mathematical calculation of addition. According to aspects of the embodiments, the software and/or computer is adapted to add the data elements on the plurality of the containers. According to aspects of the embodiments, the software and/or computer is further adapted to add a plurality of the data elements on the plurality of the containers. According to aspects of the embodiments, the software and/or computer is adapted to compare the added amount against the stored data element. According to aspects of the embodiments, the software and/or computer is configured to cross-check the added amount against the stored data element. According to aspects of the embodiments, the stored data element is patient specific. According to aspects of the embodiments, the stored data element is linked to a patient profile, a patient medication list, a patient specific prescription, an electronic prescription, a digital prescription, and/or patient administration record. According to aspects of the embodiments, the data elements on the plurality of the containers contain a dosage amount of a beneficial substance that is contained in the container. According to aspects of the embodiments, the data elements on the plurality of the containers have a dose amount of a beneficial substance that is stored in the containers. According to aspects of the embodiments, the dose amount is a volume. According to aspects of the embodiments, the dose amount is a weight. According to aspects of the embodiments, the weight is measured in micrograms, milligrams, and/or grams. According to aspects of the embodiments, the dose amount is a volume. According to aspects of the embodiments, the dose amount is a weight and a volume.

According to aspects of the embodiments, the software and/or computer is adapted to compare the plurality of the data elements of the containers against a list of stored data elements. According to aspects of the embodiments, the software is adapted to compare the plurality of the data elements disposed on the plurality of the containers intended for the modular medicament assembly (modular/modularly administered medicament) against a list of stored data elements. According to aspects of the embodiments, the list of stored data elements are patient specific. According to aspects of the embodiments, the stored data elements are linked to a patient profile, a prescription, a patient specific prescription, an electronic prescription, a digital prescription, a patient medication profile, and/or a patient administration record. According to aspects of the embodiments, the plurality of the containers further comprise a unique identifier. According to aspects of the embodiments, the unique identifier is displayed on the plurality of the containers. According to aspects of the embodiments, the unique identifier is embedded in and/or on a data element of the containers. According to aspects of the embodiments, the unique identifier is embedded in a barcode of the containers. According to aspects of the embodiments, the unique identifier is encoded in a barcode of the containers. According to aspects of the embodiments, the unique identifier is adapted to prevent repeat (double) scanning of the same vial more than once by a scanning device. According to aspects of the embodiments, the software is further configured to compare, cross-check, and/or double check the unique identifiers against each other/one another and to determine if the same container has been scanned more than once. According to aspects of the embodiments, the software and/or computer is configured to store at least one and/or a plurality of the unique identifiers and to compare/cross-check if the same unique identifier has been scanned more than once and to optionally then alert a user if a duplicate scan has or has not occurred. According to aspects of the embodiments, the software and/or computer is used in an institutional setting. According to aspects of the embodiments, the institutional setting can be a hospital, a nursing home, a clinic and combinations thereof. According to aspects of the embodiments, the software and/or computer is used in a doctor's/physician's office.

According to aspects of the embodiments, a system is provided for the verification of an amount of a beneficial substance, comprising: a plurality of containers each having a data element disposed on the plurality of the containers, the data element characterized in that it stores information that is and/or represents about a dosage amount that is inside the container; and software and/or a computer that is adapted to add the plurality of data elements from the plurality of the containers. According to aspects of the embodiments, a system is provided for the verification of an amount of a beneficial substance, comprising: a plurality of containers each having a data element disposed on the plurality of the containers, the data element characterized in that it stores information that is and/or represents a dosage amount that is (and/or that is believed to be) in the container; software and/or computer is adapted to add the plurality of data elements from the plurality of the containers, the software and/or the computer further adapted to compare the added amount against a stored data element.

According to aspects of the embodiments, a system is provided for the verification of an amount of a beneficial substance, comprising: a plurality of containers each having a data element disposed on the plurality of the containers, the data element characterized in that it stores information that is and/or represents a dosage amount that is (and/or that is believed to be) in the container; software and/or a computer is adapted to add the plurality of data elements from the plurality of the containers, the software and/or the computer further adapted to compare the added amount against a stored patient specific data element. According to aspects of the embodiments, a system is provided for the verification of beneficial substances, comprising: a plurality of containers having a data element on the containers; an acquisition device for acquiring the data elements from the plurality of the containers; and software and/or computer for comparing the data element from the plurality of containers against stored data.

According to aspects of the embodiments, a system is provided for the verification of a final amount of a medicament, comprising: a plurality of containers housing a medicament, the containers having a data element disposed on the containers, the data element characterized in that it represents about an amount of the medicament stored in the containers; and an acquisition device for acquiring the data elements from the plurality of the containers; and software and/or a computer for comparing the data element from the plurality of containers against a stored data element. According to aspects of the embodiments, a system is provided for the verification of beneficial substances, comprising: a plurality of containers having a data element on the containers; and a computer that is adapted to compare the data elements from the plurality of containers against a stored data element.

According to aspects of the embodiments, a system is provided for the verification of beneficial substances, comprising: a plurality of containers having a data element on the containers; an acquisition device for acquiring the data elements from the plurality of the containers; and a processor for comparing the data element from the plurality of containers against stored data. According to aspects of the embodiments, a system is provided for the verification of beneficial substances, comprising: a plurality of containers having a data element on the containers; and software having a processor that is adapted to add the data elements from the plurality of containers. According to aspects of the embodiments, a system is provided for the verification of beneficial substance, comprising: a plurality of containers having a data element, the plurality of containers for use for a modular medicament assembly (modularly administered medicament/MAM); an acquisition device for acquiring the data elements from the plurality of the containers; and computer for comparing the plurality of the data elements from the plurality of the containers against at least one stored data element. In one or more embodiments, the at least one stored data element is a plurality of data elements. In one or more embodiments, the at least one stored data element is at least two stored data elements, at least three stored data elements, at least four stored data elements or at least five stored data elements.

According to aspects of the embodiments, a system is provided for the verification of beneficial substances, comprising: a plurality of containers for a modularly administered medicament assembly, the plurality of the containers having a data element on the containers; an acquisition device for acquiring the data elements from the plurality of the containers; and a computer adapted to add the data elements from the plurality of containers. According to aspects of the embodiments, the computer is further configured to compare/cross-check if final dosage amount provided by the plurality of the containers is about the same as the prescribed dosage amount. According to aspects of the embodiments, a system is provided for the verification of beneficial substances, comprising: a plurality of containers for a modularly administered medicament assembly having a data element on the containers; and software adapted to add the data elements from the plurality of containers and compare the added amount against a stored data element. According to aspects of the embodiments, a system is provided for the verification of beneficial substances, comprising: a plurality of containers for a modularly administered medicament assembly, the plurality of the container having a data element; and a processor configured/adapted to add the data elements from the plurality of the containers and compare the added amount against a stored data element.

According to aspects of the embodiments, a system is provided for the verification of beneficial substances, comprising: a plurality of containers having a data element on the containers; an acquisition device for acquiring the data elements from the plurality of the containers; and a processor and/or a computer adapted to add the data elements from the plurality of containers and compare the added amount against a stored data element. According to aspects of the embodiments, a system is provided for the verification of amounts of beneficial substance, comprising: a plurality of containers having a data element on the containers; and software storing a plurality of data elements.

According to aspects of the embodiments, the software stores the plurality of the data element on the containers. According to aspects of the embodiments, a system is provided for the verification of amounts of beneficial substance, comprising: a plurality of containers having a data element disposed on the containers; and a memory element storing a plurality of data elements. According to aspects of the embodiments, the memory element stores the plurality of the data elements on the containers. According to aspects of the embodiments, a system is provided that comprises a memory element adapted to store a plurality of dosage amount data elements from a modularly assembled beneficial substance. According to aspects of the embodiments, the system stores a plurality of dosage amount data elements obtained from containers of a modular format medicament assembly (modularly administered medicament) on a patient specific profile, on a patient specific medication list, and/or on a patient specific medication administration record. According to aspects of the embodiments, the system stores a plurality of dosage amount data elements obtained from containers of a modular format medicament assembly on and/or under a patient specific screen and/or on/in a memory element of a computer and/or server. According to aspects of the embodiments, the system links a plurality of dosage amount data elements obtained from containers of a modular format medicament assembly to a patient specific identifier. According to aspects of the embodiments, the patient specific identifier is a name, a medical record number, an identification number, a date of birth, and/or a room/bed number.

According to aspects of the embodiments, a memory element is adapted to store a plurality of dosage amount data elements from a modularly assembled beneficial substance (modular format medicament assembly) intended for a specific patient. According to aspects of the embodiments, a memory element is adapted to store a plurality of dosage amount data elements linked to a patient specific identifier. According to aspects of the embodiments, a memory element is adapted to store a plurality of dosage amount data elements from a modularly assembled beneficial substance (modular format medicament assembly) linked to a patient specific identifier.

According to aspects of the embodiments, a system is provided for the verification of amounts of beneficial substance, comprising: a plurality of containers having a data element on the containers; and software storing a plurality of patient specific data elements. According to aspects of the embodiments, a system is provided for the verification of amounts of beneficial substance, comprising: a plurality of containers having a data element disposed on the containers; and a memory element storing a plurality of patient specific data elements.

According to aspects of the embodiments, a system is provided for the verification of amounts of beneficial substance, comprising: a plurality of containers having a data element on the containers; and software storing a plurality of data elements, the software is adapted to cross-check the data elements on the plurality of the containers against the plurality of data elements stored in the software. According to aspects of the embodiments, a system is provided for the verification of amounts of beneficial substance, comprising: a plurality of containers having a data element disposed on the containers; and a processor and/or a computer storing a plurality of data elements, the processor and/or the computer is adapted to cross-check the data elements of the plurality of the containers (of the modular format medicament assembly/modular administered medicament) against the plurality of data elements stored in the software. According to aspects of the embodiments, the plurality of the data elements stored in the software is linked to a patient specific identifier, a patient specific profile, a patient specific medication profile, a patient specific medication list, a patient specific administration record, and/or a patient specific medication administration record.

According to aspects of the embodiments, a system is provided for the verification of amounts of beneficial substance, comprising: a plurality of containers having a data element on the containers; and software storing a plurality of patient specific data elements, the software is adapted to cross-check the data elements of the plurality of the containers against a plurality of data elements stored in the software. According to aspects of the embodiments, a system is provided for the verification of amounts of beneficial substance, comprising: a plurality of containers having a data element disposed on the containers; and a processor and/or a computer storing a plurality of patient specific data elements, the processor is adapted to cross-check the data elements of the plurality of the containers against a plurality of data elements stored in the software.

According to aspects of the embodiments, a system is provided for the verification of amounts of a beneficial substance, comprising: a plurality of containers having a dose amount displayed on the containers; and a processor and/or a computer is adapted to perform a mathematical calculation on the plurality of dose amounts displayed on the containers and compare the mathematical calculation against at least one stored patient specific data element. According to aspects of the embodiments, a processor and/or computer is adapted to receive a plurality of data elements from a plurality of containers housing a beneficial substance and to verify if the final dosage amount of the beneficial substance in the plurality of containers is the same as the prescribed dosage amount. According to aspects of the embodiments, a processor and/or computer is adapted to receive a plurality of data elements from a modular format medicament assembly, to perform a mathematical calculation on the data elements of the modular format medicament assembly, and to verify if the final dosage amount of the beneficial substance provided by the modular format medicament assembly is the same as the prescribed dosage amount.

According to aspects of the embodiments, a processor and/or computer is adapted to add at least two data elements from the plurality of the containers. According to aspects of the embodiments, a processor and/or computer is adapted to add a plurality of data elements from a plurality of containers. According to aspects of the embodiments, the at least two and/or the plurality of the containers contain a medicament. According to aspects of the embodiments, the medicament is the same medicament. According to aspects of the embodiments, the medicament is a different medicament. According to aspects of the embodiments, the at least two and/or plurality of the containers of the modular format medicament assembly contain a nutritional substance. According to aspects of the embodiments, the nutritional substance is the same nutritional substance. According to aspects of the embodiments, the nutritional substance is a different nutritional substance.

According to aspects of the embodiments, the processor and/or computer is further adapted to receive a plurality of data elements from the plurality of the containers and add the data elements. According to aspects of the embodiments, the processor and/or computer is further adapted to compare the plurality of data elements from the plurality of the containers against a stored data element. According to aspects of the embodiments, the stored data element is a patient specific data element. According to aspects of the embodiments, the stored data element is linked to a specific patient. According to aspects of the embodiments, the stored data element is linked to a patient profile. According to aspects of the embodiments, the stored data element is linked to a patient specific medication list. According to aspects of the embodiments, the stored data element is linked to a medication administration record and/or an electronic/digital prescription.

According to aspects of the embodiments, the processor and/or computer is adapted to perform multiplication if necessary to compute a final dosage amount. According to aspects of the embodiments, the processor and/or the computer is adapted to perform addition to compute a final dosage amount. According to aspects of the embodiments, the processor and/or computer is adapted to perform addition, wherein the processor and/or the computer adds the amounts from the plurality of data elements from the plurality of the containers, determines a final dosage amount and then compares the final dosage amount against a prescribed final dosage amount. According to aspects of the embodiments, the processor and/or computer is adapted to perform addition and/or multiplication if necessary, wherein the processor and/or the computer adds and/or multiplies a list of stored data elements from the prescription/electronic prescription and then compares the final prescribed dosage amount against a final dosage amount computed (added and/or multiplied) from the plurality of the data elements from the plurality of the containers. According to aspects of the embodiments, the processor and/or computer is adapted to perform addition and/or multiplication if necessary, wherein the processor and/or the computer adds and/or multiplies a list of stored data elements from the prescription/electronic prescription and then compares the final prescribed dosage amount against a final dosage amount computed (added and/or multiplied) from the modular format medicament assembly (modular administered medicament). According to aspects of the embodiments, the processor and/or computer is adapted to perform addition and/or multiplication to compute a final dosage amount from a plurality of containers containing/housing a medicament/nutritional substance, the processor and/or computer further adapted to perform addition and/or multiplication to compute a final dosage amount from a list of stored data elements, the processor and/or the computer further configured to cross-check/compare/double-check if the final dosage amount provided by the plurality of the containers is about the same as the final dosage amount provided from the list of stored data elements. According to aspects of the embodiments, the plurality of the containers is a modular format medicament assembly (modular assembled medicament). According to aspects of the embodiments, the list of stored data elements is a prescription/electronic prescription. According to aspects of the embodiments, the electronic prescription is linked to a patient specific identifier, a patient specific profile, a patient specific medication list, and/or a patient specific medication administration record.

According to aspects of the embodiments, a processor and/or computer is adapted to: receive a plurality of data elements from a plurality of containers, wherein the data elements comprise a dosage amount; receive a plurality of stored data from a patient specific profile; and cross-check the data elements from the plurality of the containers against the plurality of stored data from the patient specific profile. According to aspects of the embodiments, a processor and/or computer is adapted to: receive a plurality of data elements, wherein the data elements comprise a dosage amount; receive a plurality of stored data from a patient specific profile; and cross-check the data elements from the plurality of the data elements against the plurality of the stored data from the patient specific profile. According to aspects of the embodiments, the patient specific profile is a patient specific medication list. According to aspects of the embodiments, the patient specific profile is a medication administration record.

According to aspects of the embodiments, a processor and/or computer is adapted to receive a plurality of data elements from a plurality of containers, wherein the data element is a dosage amount; receive a plurality of stored data from a patient specific profile; Add the data elements from the plurality of the containers; and add the data elements from the patient specific profile. According to aspects of the embodiments, the processor and/or computer is further adapted to cross-check the added amount of the data elements from the plurality of the containers against the added amount of the data elements from the patient specific profile. According to aspects of the embodiments, the plurality of the containers are a modular format medicament assembly (modular administered medicament). According to aspects of the embodiments, the patient specific profile is a patient specific medication list and/or a patient specific medication administration record.

According to aspects of the embodiments, the processor and/or computer is further adapted to produce an output. According to aspects of the embodiments, the output can be a confirmation that the final dosage amount from the plurality of the containers is the same as the final dosage amount from the plurality of dosage elements from the patient specific profile. According to aspects of the embodiments, the output can be an alert that a final dosage amount from the plurality of the containers is not the same as the final dosage amount from the plurality of the dosage elements from the patient specific profile. According to aspects of the embodiments, the output may be color coded. For example, a green color may indicate a confirmation that the final dosage amount from the plurality of the containers is the same as the final dosage amount from the plurality of the dosage elements from the patient specific profile. In another example, a red color may indicate an alert that that the final dosage amount from the plurality of the containers is not the same as the final dosage amount from the plurality of the dosage elements from the patient specific profile. According to aspects of the embodiments, the output may be a symbol. The symbol may be color coded as described above. In one example, the symbol may be a “check mark” indicating a confirmation that the final dosage amount from the plurality of the containers is the same as the final dosage amount from the plurality of the dosage elements from the patient specific profile. In another example, the symbol may be an “X” indicating an alert that the final dosage amount from the plurality of the containers is not the same as the final dosage amount from the plurality of the dosage elements from the patient specific profile. According to aspects of the embodiments, the output may be a combination of a symbol and a color. For example, the confirmation as described above may comprise a “check mark” that is green in color. In another example, the alert as described above may be an “X” that is colored red. The output may be displayed visually on a patient profile, on a patient specific profile, on a medication list, on a patient specific medication list, on a patient administration record, and/or on a patient specific medication administration record. The output may be positioned next to a drug name and/or on a particular drug's row, column, and/or field that communicates to the user of the software if a confirmation or an alert is issued for the particular drug. According to aspects of the embodiments, the output may be placed/appear on a patient specific list, patient specific profile, and/or patient specific medication administration record. According to aspects of the embodiments, the output may take the form of a sound. The sound may be an audible sound. The sound may be a pleasant sound to indicate the confirmation. The sound may be an unpleasant sound indicating the alert. According to aspects of the embodiments, the alert may be a combination of a visual cue (check mark or “X”), a color coded visual cue, a sound, and combinations thereof. The alert may be produced by a computer, by a portable device, by an smartphone, by an app, and combinations thereof.

According to aspects of the embodiments, a processor and/or computer is adapted to compare a plurality of data elements from a plurality of containers against a patient specific dosage amount. According to aspects of the embodiments, a processor and/or computer is adapted to compare a plurality of data elements from a plurality of containers against a plurality of patient specific dosage amounts. According to aspects of the embodiments, the patient specific dosage amounts are of the same medicament. According to aspects of the embodiments, the patient specific dosage amounts are for a modular assembly of beneficial substances (modular assembled medicament). According to aspects of the embodiments, the patient specific dosage amounts are for a modular format medicament assembly. According to aspects of the embodiments, the patient specific dosage amounts are for a modular format medicament assembly comprising commercially packaged and/or prepackaged containers.

According to aspects of the embodiments, a processor and/or computer is provided for cross-checking of a modular dose assembly format (modular format medicament assembly/modular administered medicament) final dosage amount against at least one stored data element. According to aspects of the embodiments, a processor and/or computer is provided for the cross-checking of a modular dose assembly format final dosage amount against a stored data element on a patient specific medication profile and/or patient specific prescription. According to aspects of the embodiments, a processor and/or computer is provided for the cross-checking of a modular dose assembly format final dosage amount against a stored data element on a patient specific medication list and/or on a prescription. According to aspects of the embodiments, a processor and/or computer is provided for the cross-checking of a modular dose assembly format final dosage amount against a stored data element on a medication administration record. According to aspects of the embodiments, a processor and/or computer is provided for the cross-checking of a modular dose assembly format final dosage amount against a stored data element on a patient specific medication administration record.

According to aspects of the embodiments, a processor and/or computer is provided for the cross-checking of a modular dose assembly format (modular format medicament assembly) final dosage amount against a stored data element on a patient specific medication administration record. According to aspects of the embodiments, a processor and/or computer is provided for the cross-checking of a modular dose assembly format (modular format medicament assembly) final dosage amount against a patient specific stored data element and/or a patient specific prescription. According to aspects of the embodiments, the software and/or the computer is adapted to add and/or multiply amounts of medicaments. According to aspects of the embodiments, the amounts are dosage amounts. According to aspects of the embodiments, the amounts are packaged amounts. According to aspects of the embodiments, the amounts are prepackaged amounts. According to aspects of the embodiments, the amounts are commercially prepackaged amounts. According to aspects of the embodiments, the amounts are commercially packaged amounts. According to aspects of the embodiments, commercially packaged amounts are packaged by a drug/pharmaceutical manufacturer.

According to aspects of the embodiments, a processor and/or computer is provided for the cross-checking of a modular dose assembly format (modular format medicament assembly) final dosage amount against a stored data element on a patient specific medication record. According to aspects of the embodiments, a processor and/or computer is provided for the cross-checking of a modular dose assembly format (modular format medicament assembly) final dosage amount against a patient specific stored data element. According to aspects of the embodiments, the processor and/or the computer is adapted to add and/or multiply amounts of medicaments. According to aspects of the embodiments, the amounts are dosage amounts. According to aspects of the embodiments, the amounts are packaged amounts. According to aspects of the embodiments, the amounts are prepackaged amounts. According to aspects of the embodiments, the amounts are commercially prepackaged amounts. According to aspects of the embodiments, the amounts are commercially packaged amounts. According to aspects of the embodiments, commercially packaged amounts are packaged by a drug/pharmaceutical manufacturer.

According to aspects of the embodiments, a processor is provided for cross-checking of a modular dose assembly format (modular format medicament assembly/modular administered medicament) final dosage amount against a stored data element stored and/or displayed on at least one of a patient specific medication list, a patient specific profile, a patient specific prescription, a patient specific electronic prescription, and a patient specific medication administration record. According to aspects of the embodiments, a computer is provided for cross-checking of a modular dose assembly format (modular format medicament assembly/modular administered medicament) final dosage amount against a stored data element stored and/or displayed on at least one of a patient specific medication list, a patient specific profile, a patient specific prescription, a patient specific electronic prescription, and a patient specific medication administration record. According to aspects of the embodiments, a processor and/or computer is provided for the cross-checking of a modular dose assembly format (modular format medicament assembly) final dosage amount against a patient specific stored data element. According to aspects of the embodiments, the processor is adapted to add and/or multiply and/or subtract amounts of medications. According to aspects of the embodiments, the computer is adapted to add and/or multiply and/or subtract amounts of medications. According to aspects of the embodiments, software is adapted to add and/or multiply amounts of medicaments. According to aspects of the embodiments, the amounts are dosage amounts. According to aspects of the embodiments, the amounts are weight amounts. According to aspects of the embodiments, the amounts are volume amounts. According to aspects of the embodiments, the amounts are weight and volume amounts. According to aspects of the embodiments, the amounts are packaged amounts. According to aspects of the embodiments, the amounts are prepackaged amounts. According to aspects of the embodiments, the amounts are commercially prepackaged amounts. According to aspects of the embodiments, the amounts are commercially packaged amounts. According to aspects of the embodiments, commercially packaged amounts are packaged by a drug/pharmaceutical manufacturer.

According to aspects of the embodiments, a processor is provided for the cross-checking of a modular dose assembly format (modular format medicament assembly/modular assembled medicament) final dosage amount against a stored data element on a patient specific medication profile. According to aspects of the embodiments, a processor is provided for the cross-checking of a modular dose assembly format (modular format medicament assembly) final dosage amount against a patient specific stored data element. According to aspects of the embodiments, software is adapted to add and/or multiply amounts of medicaments. According to aspects of the embodiments, the amounts are dosage amounts. According to aspects of the embodiments, the amounts are packaged amounts. According to aspects of the embodiments, the amounts are prepackaged amounts. According to aspects of the embodiments, the amounts are commercially prepackaged amounts. According to aspects of the embodiments, the amounts are commercially packaged amounts. According to aspects of the embodiments, commercially packaged amounts are packaged by a drug/pharmaceutical manufacturer.

According to aspects of the embodiments, a computer is provided for the cross-checking of a modular dose assembly format (modular format medicament assembly/modular assembled medicament) final dosage amount against a stored data element on a patient specific medication profile. According to aspects of the embodiments, a computer is provided for the cross-checking of a modular dose assembly format (modular format medicament assembly) final dosage amount against a patient specific stored data element. According to aspects of the embodiments, a computer is adapted to add and/or multiply amounts of medicaments. According to aspects of the embodiments, the amounts are dosage amounts. According to aspects of the embodiments, the amounts are packaged amounts. According to aspects of the embodiments, the amounts are prepackaged amounts. According to aspects of the embodiments, the amounts are commercially prepackaged amounts. According to aspects of the embodiments, the amounts are commercially packaged amounts. According to aspects of the embodiments, commercially packaged amounts are packaged by a drug/pharmaceutical manufacturer.

According to aspects of the embodiments, software is provided for the verification of amounts of beneficial substances, the software comprising a processor adapted to perform a mathematical calculation using at least one amount of a beneficial substance stored in a container. According to aspects of the embodiments, pharmacy-nursing software is adapted to receive a plurality of data elements from a plurality of containers. According to aspects of the embodiments, software is adapted to receive a plurality of data elements from a plurality of containers and adapted to store the plurality of the data elements. According to aspects of the embodiments, the pharmacy-nursing software is further adapted to store a plurality of dosages from a modular format medicament assembly.

According to aspects of the embodiments, a computer is provided for the verification of amounts of beneficial substances, the computer adapted to perform a mathematical calculation using at least one amount of a beneficial substance stored in a container. According to aspects of the embodiments, the computer is adapted to receive a plurality of data elements from a plurality of containers. According to aspects of the embodiments, the computer is adapted to receive a plurality of data elements from a plurality of containers and adapted to store the plurality of the data elements. According to aspects of the embodiments, the computer is further adapted to store a plurality of dosages from a modular format medicament assembly (modular assembled medicament).

According to aspects of the embodiments, software is adapted to receive a plurality of data elements from a plurality of containers and adapted to store the plurality of the data elements under a patient specific profile. According to aspects of the embodiments, software is adapted to receive a plurality of data elements from a plurality of containers and adapted to store the plurality of the data elements under a patient specific medication list. According to aspects of the embodiments, software is adapted to receive a plurality of data elements from a plurality of containers and adapted to store the plurality of the data elements under a patient specific medication administration record. According to aspects of the embodiments, software is adapted to receive a plurality of data elements intended for one final dose amount from a plurality of containers and adapted to store the plurality of the data elements. According to aspects of the embodiments, software is adapted to receive a plurality of data elements from a plurality of containers intended for one final dose and adapted to store the plurality of the data elements under a patient specific profile. According to aspects of the embodiments, software is adapted to receive a plurality of data elements from a plurality of containers intended for one final dose and adapted to store the plurality of the data elements under a patient specific medication list. According to aspects of the embodiments, software is adapted to receive a plurality of data elements from a plurality of containers intended for one final dose and adapted to store the plurality of the data elements under a patient specific medication administration record.

According to aspects of the embodiments, a computer is adapted to receive a plurality of data elements from a plurality of containers and adapted to store the plurality of the data elements under a patient specific profile. According to aspects of the embodiments, the computer is adapted to receive a plurality of data elements from a plurality of containers and adapted to store the plurality of the data elements under a patient specific medication list. According to aspects of the embodiments, the computer is adapted to receive a plurality of data elements from a plurality of containers and adapted to store the plurality of the data elements under a patient specific medication administration record. According to aspects of the embodiments, the computer is adapted to receive a plurality of data elements intended for one final dose amount from a plurality of containers and adapted to store the plurality of the data elements. According to aspects of the embodiments, a computer is adapted to receive a plurality of data elements from a plurality of containers intended for one final dose and adapted to store the plurality of the data elements under a patient specific profile. According to aspects of the embodiments, a computer is adapted to receive a plurality of data elements from a plurality of containers intended for one final dose and adapted to store the plurality of the data elements under a patient specific medication list. According to aspects of the embodiments, a computer is adapted to receive a plurality of data elements from a plurality of containers intended for one final dose and adapted to store the plurality of the data elements under a patient specific medication administration record.

According to aspects of the embodiments, a processor and/or a computer is adapted to receive a plurality of dosage amount information from a plurality of containers, compute a final dosage amount provided by the plurality of the containers, and compare the final dosage amount provided by the plurality of the containers against a second stored patient specific final dosage amount. According to aspects of the embodiments, a processor and/or computer is adapted to receive a plurality of dosage amount information from a plurality of containers housing a medicament, the processor further adapted to receive a plurality of dosage amount information from a storage element. According to aspects of the embodiments, the storage element is located in a computer. According to aspects of the embodiments, the storage element is located on a portable device. According to aspects of the embodiments, the portable device is a handheld device. According to aspects of the embodiments, the handheld device is a smartphone. According to aspects of the embodiments, the storage element is a server. According to aspects of the embodiments, the storage element is in the cloud. According to aspects of the embodiments, the storage element is encrypted. According to aspects of the embodiments, the plurality of the containers are a modular format medicament assembly. According to aspects of the embodiments, the modular format medicament assembly provides a customized final dose of a beneficial substance/medicament/nutritional substance in a commercially packaged/prepackaged format.

According to aspects of the embodiments, a computer is adapted to receive a plurality of dosage amount information from a plurality of containers housing a medicament, the computer further adapted to receive a plurality of dosage amount information from a storage element, the computer further adapted to cross-check the plurality of the dosage amount information from the plurality of containers against the plurality of dosage amount information from the storage element. According to aspects of the embodiments, a computer is adapted to: receive a plurality of dosage amount information from a plurality of containers housing a beneficial substance; receive a plurality of dosage amount information from a storage element; compute a final dosage amount from the plurality of the containers; and cross-check the plurality of the dosage amount information from the storage element against the final dosage amount from the plurality of the containers. According to aspects of the embodiments, the computing involves at least one of addition, multiplication, and subtraction. According to aspects of the embodiments, the computing involves at least one of addition, multiplication, subtraction, and division.

According to aspects of the embodiments, a computer is adapted to receive a plurality of dosage amount information from a plurality of containers housing a beneficial substance; receive a plurality of dosage amount information from a storage element; compute a final dosage amount from the plurality of dosage amount information from the storage element; and cross-check the final dosage amount from the storage element against the plurality of dosage amount information from the plurality of the containers. According to aspects of the embodiments, a computer is adapted to: receive a plurality of dosage amount information from a plurality of containers housing a beneficial substance; receive a plurality of dosage amount information from a storage element; compute a final dosage amount from the plurality of the containers; compute a final dosage amount from the plurality of dosage amount information from the storage element; and cross-check the final dosage amount from the plurality of the containers against the final dosage amount from the storage element. According to aspects of the embodiments, the computing involves at least one of addition, multiplication, subtraction, and division.

According to aspects of the embodiments, software is provided for the verification of at least two amounts of a beneficial substance, the software is adapted to perform a mathematical calculation on at least two data elements provided to the software. According to aspects of the embodiments, software is provided for the verification of an amount of a beneficial substance provided in a modular format, the software comprising: means to add at least two data elements, wherein at least two of the at least two data elements are an amount of a beneficial substance housed in a container. According to aspects of the embodiments, software is adapted to add at least two dosage amounts. According to aspects of the embodiments, software is adapted to add a plurality of dosage amounts. According to aspects of the embodiments, software is adapted to multiply at least two dosage amounts. According to aspects of the embodiments, software is adapted to multiply a plurality of dosage amounts. According to aspects of the embodiments, software having a processor is adapted to add at least two dosage amounts. According to aspects of the embodiments, software having means adapted to add a plurality of dosage amounts supplied to an end-user and/or patient. According to aspects of the embodiments, software having means is adapted to multiply a plurality of dosage amounts. According to aspects of the embodiments, software having means to multiply a plurality of dosage amounts supplied to an end-user and/or patient.

According to aspects of the embodiments, the plurality of the dosage amounts are from a modular dosage assembly (modular format medicament assembly). According to aspects of the embodiments, the modular dosage assembly (modular format medicament assembly) is patient specific. According to aspects of the embodiments, the modular dosage assembly (modular format medicament assembly) is non-patient specific. According to aspects of the embodiments, the modular dosage assembly comprises commercially packaged containers. According to aspects of the embodiments, the modular dosage assembly comprises commercially prepackaged containers.

According to aspects of the embodiments, software having means to multiply at least two dosage amounts. According to aspects of the embodiments, software is adapted to multiply a plurality of dosage amounts. According to aspects of the embodiments, a processor is adapted to compare a plurality of dosage amounts from a plurality of containers against a list of a plurality of dosage amounts. According to aspects of the embodiments, the list is a predetermined list. According to aspects of the embodiments, the list is a patient specific list. According to aspects of the embodiments, the list is linked to a patient specific profile. According to aspects of the embodiments, the list is linked to a patient specific medication list. According to aspects of the embodiments, the list is linked to a medication administration record. According to aspects of the embodiments, the list is linked to a patient specific medication administration record. According to aspects of the embodiments, the list is displayed on a patient profile. According to aspects of the embodiments, the list is displayed on a patient specific profile. According to aspects of the embodiments, the list is displayed on a patient administration record. According to aspects of the embodiments, the list is displayed on a patient specific medication administration record. According to aspects of the embodiments, the link is an electronic link.

According to aspects of the embodiments, a computer adapted to compare a plurality of dosage amounts from a plurality of containers against a list of a plurality of dosage amounts stored under a patient specific profile. According to aspects of the embodiments, the list is a predetermined list. According to aspects of the embodiments, the list is a patient specific list. According to aspects of the embodiments, the list is linked to a patient specific profile. According to aspects of the embodiments, the list is linked to a patient specific medication list. According to aspects of the embodiments, the list is linked to a medication administration record. According to aspects of the embodiments, the list is linked to a patient specific medication administration record. According to aspects of the embodiments, the list is displayed on a patient profile. According to aspects of the embodiments, the list is displayed on a patient specific profile. According to aspects of the embodiments, the list is displayed on a patient administration record. According to aspects of the embodiments, the list is displayed on a patient specific medication administration record. According to aspects of the embodiments, the plurality of dosage amounts are a modular format medicament assembly.

According to aspects of the embodiments, a processor and/or a computer is adapted to compare a plurality of dosage amounts from a plurality of containers against a list of a plurality of dosage amounts stored under a patient specific medication administration record. According to aspects of the embodiments, a processor and/or a computer is adapted to cross-check a plurality of dosage amounts from a plurality of containers against a list of a plurality of dosage amounts. According to aspects of the embodiments, a processor and/or a computer is adapted to cross-check a plurality of dosage amounts from a plurality of containers against a list of a plurality of dosage amounts stored under a patient specific medication administration record. According to aspects of the embodiments, a computer adapted to cross-check a plurality of dosage amounts obtained from a plurality of containers against a list of a plurality of dosage amounts stored and/or linked to a prescription. According to aspects of the embodiments, the plurality of dosage amounts are a modular format medicament assembly. According to aspects of the embodiments, the prescription is an electronic prescription. According to aspects of the embodiments, the prescription is a digital prescription. According to aspects of the embodiments, the prescription is a digitally stored prescription.

According to aspects of the embodiments, a processor is adapted to receive a plurality of dosage amount information from a plurality of containers, compute a final dosage amount provided by the plurality of the containers, and compare the final dosage amount provided by the plurality of the containers against a second final dosage amount. According to aspects of the embodiments, a processor is adapted to receive a plurality of dosage amount information from a plurality of containers, compute a final dosage amount provided by the plurality of the containers, and compare the final dosage amount provided by the plurality of the containers against a second stored final dosage amount.

According to aspects of the embodiments, a computer is adapted to receive a plurality of dosage amount information from a plurality of containers, compute a final dosage amount provided by the plurality of the containers, and compare the final dosage amount provided by the plurality of the containers against a second final dosage amount. According to aspects of the embodiments, a computer is adapted to receive a plurality of dosage amount information from a plurality of containers, compute a final dosage amount provided by the plurality of the containers, and compare the final dosage amount provided by the plurality of the containers against a second stored final dosage amount.

According to aspects of the embodiments, a processor is adapted to receive a plurality of dosage amount information from a plurality of containers, compute a final dosage amount provided by the plurality of the containers, and compare the final dosage amount provided by the plurality of the containers against a second patient specific final dosage amount. According to aspects of the embodiments, a processor is adapted to: receive a plurality of data elements, the data elements having dose amount information; compute a final dosage amount using the plurality of the data elements; and display the final dosage amount on a screen

According to aspects of the embodiments, a computer is adapted to receive a plurality of dosage amount information from a plurality of containers, compute a final dosage amount provided by the plurality of the containers, and compare the final dosage amount provided by the plurality of the containers against a second patient specific final dosage amount. According to aspects of the embodiments, a computer is adapted to: receive a plurality of data elements, the data elements having dose amount information; compute a final dosage amount using the plurality of the data elements; and display the final dosage amount on a screen.

According to aspects of the embodiments, a processor is adapted to: receive a plurality of data elements, the data elements having dose amount information; compute a final dosage amount using the plurality of the data elements; and display the final dosage amount on a patient specific profile. According to aspects of the embodiments, the patient specific profile is a medication profile. According to aspects of the embodiments, the patient specific profile is a medication administration record. According to aspects of the embodiments, the patient specific profile is a patient specific medication list. According to aspects of the embodiments, the final dosage amount is stored on the patient specific profile. According to aspects of the embodiments, the final dosage amount is stored on the medication administration record. According to aspects of the embodiments, the final dosage amount is stored on a patient specific medication list.

According to aspects of the embodiments, a computer is adapted to: receive a plurality of data elements, the data elements having dose amount information; compute a final dosage amount using the plurality of the data elements; and display the final dosage amount on a patient specific profile. According to aspects of the embodiments, the patient specific profile is a medication profile. According to aspects of the embodiments, the patient specific profile is a medication administration record. According to aspects of the embodiments, the patient specific profile is a patient specific medication list. According to aspects of the embodiments, the final dosage amount is stored on the patient specific profile. According to aspects of the embodiments, the final dosage amount is stored on the medication administration record. According to aspects of the embodiments, the final dosage amount is stored on a patient specific medication list.

According to aspects of the embodiments, software is provided for the verification of at least two amounts of a beneficial substance, the software is adapted to perform a mathematical calculation on at least two data elements provided to the software, the two data elements originating from barcode(s) on containers containing a beneficial substance(s). According to aspects of the embodiments, the beneficial substance(s) are medicaments. According to aspects of the embodiments, software is provided for the verification of an amount of a beneficial substance provided in a modular format.

According to aspects of the embodiments, a processor is adapted to add at least two data elements, wherein at least two of the at least two data elements are an amount of a beneficial substance housed in a container. According to aspects of the embodiments, software is adapted to add at least two dosage amounts, the dosage amount information provided to the software via a scanning elements disposed on containers housing medicament(s). According to aspects of the embodiments, software is adapted to add a plurality of dosage amounts housed in commercially packaged containers. According to aspects of the embodiments, software is adapted to multiply at least two dosage amounts. According to aspects of the embodiments, software is adapted to multiply a plurality of dosage amounts. According to aspects of the embodiments, software having a processor is adapted to add at least two dosage amounts. According to aspects of the embodiments, software having a processor is adapted to add a plurality of dosage amounts. According to aspects of the embodiments, software having a processor is adapted to multiply at least two dosage amounts. According to aspects of the embodiments, software is adapted to multiply a plurality of dosage amounts.

According to aspects of the embodiments, a computer is adapted to add at least two data elements, wherein at least two of the at least two data elements are an amount of a beneficial substance housed in a container. According to aspects of the embodiments, a computer is adapted to add at least two dosage amounts, the dosage amount information provided to the software via a scanning elements disposed on containers housing medicament(s). According to aspects of the embodiments, a computer is adapted to add a plurality of dosage amounts housed in commercially packaged containers. According to aspects of the embodiments, computer is adapted to multiply at least two dosage amounts. According to aspects of the embodiments, a computer is adapted to multiply a plurality of dosage amounts. According to aspects of the embodiments, a computer is adapted to add at least two dosage amounts. According to aspects of the embodiments, a computer is adapted to add a plurality of dosage amounts. According to aspects of the embodiments, a computer is adapted to multiply at least two dosage amounts. According to aspects of the embodiments, a computer is adapted to multiply a plurality of dosage amounts.

Aspects of the embodiments are based on containers containing beneficial substances having unique identifiers. According to aspects of the embodiments, the unique identifiers can differentiate two separate vials, wherein each of the two separate vials contains the same beneficial substance. According to aspects of the embodiments, the unique identifiers can differentiate two separate vials, wherein each of the two separate vials contains the same beneficial substance in about the same amount. According to aspects of the embodiments, the unique identifiers can differentiate two separate vials, wherein each of the two separate vials contains the same beneficial substance in the same amount. According to aspects of the embodiments, the unique identifiers can differentiate two separate vials, wherein each of the two separate vials contains the same beneficial substance in the same labeled amount. According to aspects of the embodiments, the beneficial substance is a medicament and the unique identifiers can differentiate at least two vials housing the same medicament in the same amount. By way of non-limiting example, Vial “A” contains penicillin 5 million units and Vial “B” contains penicillin 5 million units. The unique identifier that can be disposed on Vial “A” and/or Vial “B” can identify Vial “A” as being different from Vial “B”. The unique identifiers mentioned can be displayed on the mentioned vials and visible to a user, the unique identifiers can be in the form of a barcode printed and/or affixed to Vial “A” and/or Vial “B”, the unique identifiers can be numeric, alphabetic, alpha-number and or in any other configuration/form. The unique identifiers are adapted to prevent double (repeat) scanning of the same vial by a user. By way of non-limiting example, a modularly assembled final dosage is provided to a nurse in the following format:
Bag containing Dextrose 5% 100 milliliters, the bag having at least two entry ports adapted to engage and accept the contents of Vial “A” and of Vial “B”;
Vial “A” containing Penicillin 5 million units, vial “A” having a unique identifier information embedded in a barcode; and
Vial “B” containing Penicillin 5 million units, vial “B” having a unique identifier information embedded in a barcode.

Vial “A” can be attached to one entry port of the Bag, Vial “B” can then be attached to a second entry port of the bag. According to aspects of the embodiments, a modularly assembled final dosage amount of Penicillin 10 million units is attached to a Dextrose 5% 100 milliliter bag. The end-user/nurse scans (using a scanning device) the barcode of Vial “A”, then the end-user/nurse mistakenly scans Vial “A” again (when nurse should have scanned Vial “B”), the unique identifier embedded in the barcode of Vial “A” alerts the computer (and/or the processor) and/or the software program that the same vial has been scanned more than one time. The software can in some embodiments, reject the double scan of Vial “A” since it is a double (duplicate) scan. The software/processor can alert the user/nurse that a double scan has occurred and that this is an error. The alert can be audible such as a noise, visual (optionally color coded) such as a marking on a screen, patient profile, and/or patient administration record, and/or in other forms. If the nurse scanned Vial “A” and then Vial “B”, then the unique identifiers on each vial would inform the computer/processor/software that Vial “A” and Vial “B” are different vials and the software/processor would allow the end-user/nurse to proceed with administration of the modularly assembled final dosage amount and may optionally provide an output such as an audible noise, visual (optionally color coded) such as a marking on a screen, patient profile, and/or patient administration record, and/or in other forms alerting the user that it is safe to proceed with administration of the final product (dosage amount). According to aspects of the embodiments, the mentioned vials can be syringes or other types of containers.

According to aspects of the embodiments, a plurality of containers housing a beneficial substance is provided, wherein each of the containers has a unique identifier, the unique identifier having at least one of a beneficial substance name and a beneficial substance amount. According to aspects of the embodiments, the beneficial substance is a medicament. According to aspects of the embodiments, the beneficial substance is a nutritional substance such as (but not limited to) an amino acid, a protein, a lipid, a fat, a vitamin, a multivitamin, a trace element, a mineral, etc. According to aspects of the embodiments, the beneficial substance is the same beneficial substance. According to aspects of the embodiments, the beneficial substance is the same beneficial substance in about the same amount. According to aspects of the embodiments, the beneficial substance is the same beneficial substance in the same amount. According to aspects of the embodiments, the beneficial substance is the same beneficial substance in the same labeled amount. According to aspects of the embodiments, the beneficial substance is a medicament in about the same amount. According to aspects of the embodiments, the beneficial substance is a medicament in the same amount. According to aspects of the embodiments, the beneficial substance is a medicament in the same labeled amount.

According to aspects of the embodiments, a plurality of containers housing the same medicament in about the same amount, wherein each of the containers having a unique identification code. According to aspects of the embodiments, the unique identification code differentiates each of the containers from the other containers. According to aspects of the embodiments, a plurality of containers housing the same medicament in the same amount, wherein each of the containers having a unique identification code. According to aspects of the embodiments, the unique identification code differentiates each of the containers from the other containers. According to aspects of the embodiments, a plurality of containers housing the same medicament in the same labeled amount, wherein each of the containers having a unique identification code. According to aspects of the embodiments, the unique identification code differentiates each of the containers from the other containers. For example, a box supplied by a drug company (pharmaceutical manufacturer) contains 10 commercially packaged/prepackaged vials, wherein each vial contains Penicillin 5 million units. Each of the 10 vials contains the same drug in about the same amount (or in the same amount, or in the same labeled amount). Each of the 10 vials having a unique identification code on the vial which differentiates each vial from the other vials in the box. The unique identification code may be a number, alphabetic, alpha-number, embedded in a barcode, embedded on a barcode, embedded in a barcode, and/or configured to be read/scanned by an acquisition device such as a scanner (optionally, handheld scanner).

According to aspects of the embodiments, a plurality of containers housing the same medicament, wherein the plurality of containers are labeled as containing the same amount of the medicament, wherein each of the containers has a unique identifier. By way of a non-limiting example, a box having ten penicillin 5 million unit vials from the same manufacturer is provided, each of the ten penicillin 5 million unit vials can have a unique identifier and/or a unique identification code that is different and/or distinguishes each vial from another vial in the box of ten vials. According to aspects of the embodiments, by way of a non-limiting example, a box having ten penicillin 5 million unit vials is provided from a first manufacturer and a box of ten penicillin 5 million unit vials is provided from a second manufacturer. Each of the ten penicillin 5 million unit vials from the first manufacturer can have a unique identifier and/or a unique identification code that is different and/or distinguishes each of the ten vials from the first manufacturer from each of the ten vials from the second manufacturer. According to aspects of the embodiments, by way of a non-limiting example, each of the ten 5 million units vials from the first manufacturer and each of the ten 5 million unit vials from the second manufacturer may each have a unique identifier and/or unique identification code that is different and/or distinguishes each of the vials from any other vial containing penicillin 5 million units (for example, out of 20 total vials, 10 vials from the first manufacturer plus 10 vials from the second manufacturer, each 1 vial has a unique identifier and/or unique identification code that is different and/or distinguishes each of the vials from the other 19 vials from either the first and/or second manufacturer). According to aspects of the embodiments, more than two manufacturers for the same drug and same amount are contemplated. In one non-limiting example 3 or more manufacturers may produce/manufacture the same drug in the same amount, optionally about the same amount, wherein each vial/container from 3 or more manufacturers has a unique identifier and/or a unique identification code that is different and/or distinguishes each of the vials from another vial. In another non-limiting example, at least 4 manufacturers may produce/manufacture the same drug in the same amount, optionally about the same amount, wherein each vial/container from 4 or more manufacturers has a unique identifier and/or a unique identification code that is different and/or distinguishes each of the vials from another vial irrespective of the drug manufacturer. The above examples illustrate unique identifiers/unique identification codes as becoming an industry standard wherein no two drug manufacturers will use the same unique identifiers/unique identification codes for their products.

According to aspects of the embodiments, a container having a unique identification code disposed on the container. According to aspects of the embodiments, a container having a unique identification code disposed in the container. Aspects of the embodiments provide a container having a unique identification code disposed on a container label. According to aspects of the embodiments, a plurality of containers having/containing the same beneficial substance, the containers having a unique identifier. According to aspects of the embodiments, a plurality of containers storing the same beneficial substance in about the same amount, the containers having a unique identifier. According to aspects of the embodiments, a plurality of containers storing the same beneficial substance in the same amount, the containers having a unique identifier. According to aspects of the embodiments, a plurality of containers storing the same beneficial substance in about the same labeled amount, the containers having a unique identifier. According to aspects of the embodiments, a plurality of containers storing the same beneficial substance in the same labeled amount, the containers having a unique identifier.

According to aspects of the embodiments, a plurality of containers is provided housing the same beneficial substance in the same amount, the containers having a unique identifier. According to aspects of the embodiments, a plurality of containers is provided housing the same medicament, the containers labeled as housing the same amount of the medicament, wherein a plurality of the containers have a unique identifier. According to aspects of the embodiments, a plurality of containers is provided housing the same medicament, the containers labeled as housing the same amount of the medicament, wherein each of the containers has a unique identifier.

According to aspects of the embodiments, a system is provided for scanning a plurality of containers containing a beneficial substance, comprising: a plurality of containers, the containers having a unique identifier; and a scanning device adapted to receive the unique identifiers. According to aspects of the embodiments, a system is provided for scanning a plurality of containers containing a beneficial substance, comprising: a plurality of containers, the containers having a unique identifier; and a processor is adapted to receive at least two unique identifiers, the processor further adapted to determine if the at least two unique identifiers are the same. According to aspects of the embodiments, a system is provided for scanning a plurality of containers containing a beneficial substance, comprising: a plurality of containers, the containers having a unique identifier; and a computer adapted to receive at least two unique identifier information, the computer further adapted to determine if the at least two unique identifiers are the same. According to aspects of the embodiments, the computer further adapted to determine if the at least two unique identifiers are different. According to aspects of the embodiments, the computer further adapted to produce an output alerting a user if the at least two unique identifiers are the same or are different.

According to aspects of the embodiments, a system for scanning a plurality of containers containing a beneficial substance, comprising: a plurality of containers, the containers having a unique identifier; and a processor is adapted to receive at least two unique identifiers, the processor further adapted to determine if the at least two unique identifiers are different. According to aspects of the embodiments, a system is provided for scanning a plurality of containers containing a beneficial substance, comprising: a plurality of containers, the containers having a unique identifier; and a processor is adapted to receive the plurality of the unique identifiers, the processor further adapted to determine if the plurality of the unique identifiers are the same.

According to aspects of the embodiments, a system for scanning a plurality of containers containing a beneficial substance, comprising a plurality of containers, the containers having a unique identifier; and a computer adapted to receive at least two unique identifiers, the computer further adapted to determine if the at least two unique identifiers are different. According to aspects of the embodiments, a system is provided for scanning a plurality of containers containing a beneficial substance, comprising: a plurality of containers, the containers having a unique identifier; and a computer adapted to receive the plurality of the unique identifiers, the computer further adapted to determine if the plurality of the unique identifiers are the same. The computer still further adapted to determine if the plurality of the unique identifiers are different.

According to aspects of the embodiments, a system for scanning a plurality of containers containing a beneficial substance, comprising: a plurality of containers, the containers having a unique identifier; and a processor is adapted to receive the plurality of the unique identifiers, the processor further adapted to determine if the plurality of the unique identifiers are different. According to aspects of the embodiments, a system for scanning a plurality of containers containing a beneficial substance, comprising: a plurality of containers, the containers having a unique identifier; and a processor is adapted to receive the plurality of the unique identifiers, the processor further adapted to compare the plurality of unique identifiers against one another to determine if any two unique identifiers are the same.

According to aspects of the embodiments, a system for scanning a plurality of containers containing a beneficial substance for a modular assembled medicament, the system comprising: a plurality of containers, the containers having a unique identifier; and a computer adapted to receive the plurality of the unique identifiers, the computer further adapted to determine if the plurality of the unique identifiers are different. According to aspects of the embodiments, a system for scanning a plurality of containers containing a beneficial substance for a modular assembled medicament, the system comprising: a plurality of containers, the containers having a unique identifier; and a computer adapted to receive the plurality of the unique identifier, the computer further adapted to compare the plurality of the unique identifiers against one another to determine if any two unique identifiers are the same.

According to aspects of the embodiments, a system for scanning a plurality of containers containing a beneficial substance, comprising: a plurality of containers, the containers having a unique identifier; and a processor is adapted to receive the plurality of the unique identifiers, the processor further adapted to compare the plurality of unique identifiers against one another to determine if any of the unique identifiers are different. According to aspects of the embodiments, the plurality of the containers are configured for a modularly assembled medicament.

According to aspects of the embodiments, a system for scanning a plurality of containers containing a beneficial substance, comprising: a plurality of containers, the containers having a unique identifier; and a computer adapted to receive the plurality of the unique identifiers, the computer further adapted to compare the plurality of the unique identifiers against one another to determine if any of the unique identifiers are different. According to aspects of the embodiments, the plurality of the containers are characterized in that they are for a modularly assembled medicament.

According to aspects of the embodiments, a system for scanning a plurality of containers containing a beneficial substance, comprising: a plurality of containers, the containers having a unique identifier; and software adapted to receive the plurality of the unique identifiers, the software further adapted to compare the plurality of the unique identifiers against one another to determine if any of the unique identifiers are different. According to aspects of the embodiments, the plurality of the containers are characterized in that they are for a modularly assembled medicament.

According to aspects of the embodiments, a system for scanning a plurality of containers containing a beneficial substance, comprising: a plurality of containers, the containers having a unique identifier; and a processor and/or a computer adapted to receive the plurality of the unique identifiers, the processor and/or computer further adapted to compare the plurality of unique identifiers against one another to determine if any of the unique identifiers are identical. According to aspects of the embodiments, a system for scanning a plurality of containers containing a beneficial substance, comprising: a plurality of containers, the containers having a unique identifier; a processor and/or a computer adapted to receive the plurality of the unique identifiers, the processor and/or computer further adapted to compare the plurality of unique identifiers against one another to determine if any of the unique identifiers are identical, the processor and/or computer further adapted to provide an output alerting a user if the same container has been scanned at least twice. According to aspects of the embodiments, a system for scanning a plurality of containers containing a beneficial substance, comprising: a plurality of containers, the containers having a unique identifier; a computer is adapted to receive the plurality of the unique identifiers, the computer further adapted to compare the plurality of unique identifiers against one another to determine if any of the unique identifiers are identical, the computer further adapted to provide an output alerting a user if the same container has been scanned at least twice or alerting a user if the same container has not been scanned twice.

According to aspects of the embodiments, a system is provided for scanning a plurality of containers containing a beneficial substance, comprising: a plurality of containers, the containers having a unique identifier; a processor is adapted to receive the plurality of the unique identifiers, the processor further adapted to compare the plurality of unique identifiers against one another to determine if any of the unique identifiers are identical, the processor further adapted to provide an output alerting a user if it is safe to proceed with administration of the beneficial substance to a patient or if it is not safe to proceed with administration of the beneficial substance to a patient.

According to aspects of the embodiments, a system is provided for scanning a plurality of containers containing a beneficial substance for a modular assembled medicament, comprising: a plurality of containers, the containers having a unique identifier, a computer adapted to receive the plurality of the unique identifiers, the computer further adapted to compare the plurality of the unique identifiers against one another to determine if any of the unique identifiers are identical, the computer further adapted to provide an output alerting a user if it is safe to proceed with administration of the beneficial substance to a patient or if it is not safe to proceed with administration of the beneficial substance to a patient.

According to aspects of the embodiments, a system is provided for scanning a plurality of containers containing a beneficial substance, comprising: a plurality of containers, the containers having a unique identifier; a processor is adapted to receive the plurality of the unique identifiers, the processor further adapted to compare the plurality of the unique identifiers against one another to determine if any of the unique identifiers are identical, the processor further adapted to provide an output alerting a user if it is safe to proceed with administration of the beneficial substance to a patient or if it is not safe to proceed with administration of the beneficial substance to a patient, the processor further adapted to send the output to a storage element and/or a memory element.

According to aspects of the embodiments, a system is provided for scanning a plurality of containers containing a beneficial substance for a modular assembled medicament, comprising: a plurality of containers, the containers having a unique identifier; a computer adapted to receive the plurality of the unique identifiers, the computer further adapted to compare the plurality of the unique identifiers against one another to determine if any of the unique identifiers are identical, the computer further adapted to provide an output alerting a user if it is safe to proceed with administration of the beneficial substance to a patient or if it is not safe to proceed with administration of the beneficial substance to a patient, the computer further adapted to send the output to a storage element and/or a memory element.

According to aspects of the embodiments, a system is provided for scanning a plurality of containers containing a beneficial substance, comprising: a plurality of containers, the containers having a unique identifier; a processor adapted to receive the plurality of the unique identifiers, the processor further adapted to compare the plurality of unique identifiers against one another to determine if any of the unique identifiers are identical, the processor further adapted to provide a visual and/or audible output alerting a user if it is safe to proceed with administration of the beneficial substance to a patient or if it is not safe to proceed with administration of the beneficial substance to a patient.

According to aspects of the embodiments, a system is provided for scanning a plurality of containers containing a beneficial substance, comprising: a plurality of containers, the containers having a unique identification code; a computer adapted to receive the plurality of the unique identification codes, the computer further adapted to compare the plurality of the unique identification codes against one another to determine if any of the unique identification codes are identical, the computer further adapted to provide a visual and/or audible output alerting a user if it is safe to proceed with administration of the beneficial substance to a patient or if it is not safe to proceed with administration of the beneficial substance to a patient.

According to aspects of the embodiments, a system is provided for scanning a plurality of containers containing a beneficial substance, comprising: a plurality of containers for a modularly assembled medicament, the plurality of the container having a unique identifier; software adapted to receive the plurality of the unique identifiers, the software further adapted to compare the plurality of the unique identifiers against each other to determine if any of the unique identifiers are identical. According to aspects of the embodiments, the software further configured to provide an output alerting a user if any of the two unique identifiers are the same. According to aspects of the embodiments, the software further configured to provide a visual and/or audible output alerting a user if it is safe to proceed with administration of the beneficial substance to a patient or if it is not safe to proceed with administration of the beneficial substance to a patient.

According to aspects of the embodiments, a system is provided for scanning a plurality of containers containing a beneficial substance, comprising: a plurality of containers, the containers having a unique identifier; a computer adapted to receive the plurality of the unique identifiers, the computer further adapted to compare the plurality of unique identifiers against one another to determine if any of the unique identifiers are identical, the computer further adapted to provide an output, the output characterized in that the output is displayed on a patient specific profile, on a patient specific medication profile, on a patient specific medication list, on a non-patient specific display, on a non-patient specific screen, and/or on a patient specific medication administration record and/or combinations thereof. According to aspects of the embodiments, the output contains information if any of the unique identifiers are the same. According to aspects of the embodiments, the output contains information if any of the unique identifiers are different.

According to aspects of the embodiments, a system is provided for scanning a plurality of containers containing a beneficial substance, comprising: a plurality of containers, the containers having a unique identifier; a computer adapted to receive the plurality of the unique identifiers, the computer further adapted to compare the plurality of unique identifiers against one another to determine if any of the unique identifiers are identical, the computer further adapted to provide an output, the output characterized in that the output is stored on a patient specific profile, on a patient specific medication profile, on a patient specific medication list, and/or on a patient specific medication administration record, on a memory element, in a memory element, on a memory element, and/or combinations thereof. According to aspects of the embodiments, the output contains information if any of the unique identifiers are the same. According to aspects of the embodiments, the output contains information if any of the unique identifiers are different. According to aspects of the embodiments, the stored output contains the unique identifier. According to aspects of the embodiments, the stored output contains the unique identifier code. According to aspects of the embodiments, the stored output contains the unique identification code. According to aspects of the embodiments, the stored output contains the unique identifier linked to a patient specific identifier. According to aspects of the embodiments, the stored output contains the unique identification code linked to a patient specific identifier. According to aspects of the embodiments, the patient specific identifier is a patient's name, medical record number, social security number, date of birth, registration number, hospital registration number, bed number, room number, and combinations thereof.

According to aspects of the embodiments, a system is provided for scanning a plurality of containers containing a beneficial substance, comprising: a plurality of containers, the containers having a unique identifier; a processor and/or computer adapted to receive the plurality of the unique identifiers, the processor and/or computer further adapted to compare the plurality of unique identifiers against one another to determine if any of the unique identifiers are identical, the processor and/or computer further adapted to provide an output, the output characterized in that the output is displayed and stored on a patient specific profile, on a patient specific medication profile, on a patient specific medication list, on/in a prescription, and/or on a patient specific medication administration record and/or combinations thereof.

According to aspects of the embodiments, a system is provided for scanning a plurality of containers containing a beneficial substance, comprising: a plurality of containers, the containers having a unique identifier; a computer adapted to receive the plurality of the unique identifiers, the computer further adapted to compare the plurality of unique identifiers against one another to determine if any of the unique identifiers are identical, the computer further adapted to provide an output, the output characterized in that the output is linked to a patient specific profile, a patient specific medication profile, a patient specific medication list, and/or a patient specific medication administration record and/or combinations thereof.

According to aspects of the embodiments, a processing device is provided for identifying repeat scanning of the same identifiers, comprising: a processing device adapted to receive at least two unique identifiers, the processing device further adapted to determine if the at least two unique identifiers are the same. According to aspects of the embodiments, a processing device is provided for identifying repeat scanning of the same identifiers, comprising: a processing device adapted to receive at least two unique identifiers, the processing device further adapted to determine if the at least two unique identifiers are different. According to aspects of the embodiments, the identifiers are disposed on containers of a modular format medicament assembly. According to aspects of the embodiments, the identifiers are printed on and/or attached to containers used for a modular format medicament assembly. According to aspects of the embodiments, the identifiers are embedded in a barcode disposed on containers. According to aspects of the embodiments, the unique identifiers are encoded on a barcode of the containers. According to aspects of the embodiments, the processing device is selected from a computer, a smartphone, a tablet, and combinations thereof. According to aspects of the embodiments, the processing device is a server.

According to aspects of the embodiments, a processor is provided for identifying repeat scanning of the same identifiers, comprising: a processor adapted to receive at least two unique identifiers from at least two containers containing an injectable medicament, the processor further adapted to determine if the at least two unique identifiers are the same. According to aspects of the embodiments, a processor is provided for identifying repeat scanning of the same identifiers, comprising: a processor is adapted to receive at least two unique identifiers from at least two containers containing an injectable medicament, the processor further adapted to determine if the at least two unique identifiers are different.

According to aspects of the embodiments, a processor is provided for identifying repeat scanning of the same identifiers, comprising: a processor adapted to receive at least two unique identifiers from at least two containers containing an injectable medicament, the processor further adapted to check if any of the at least two unique identifiers are the same. According to aspects of the embodiments, a processor is provided for identifying repeat scanning of the same identifiers, comprising: a processor is adapted to receive at least two unique identifiers from at least two containers containing an injectable medicament, the processor further adapted to check if any of the at least two unique identifiers are different.

According to aspects of the embodiments, a computer is provided for identifying repeat scanning of the same identifiers, comprising: a computer adapted to receive at least two unique identifiers from at least two containers containing an injectable medicament, the computer further adapted to check if any of the at least two unique identifiers are the same, the computer further adapted to send an output alerting a user if any of the at least two unique identifiers are the same.

According to aspects of the embodiments, software is provided for identifying duplicate scanning of the same unique identifiers, comprising: software adapted to receive at least two unique identifiers from at least two containers containing an intracorporeally administered medicament, the software further adapted to compare if any of the at least two unique identifiers are the same, the software further adapted to send an output alerting a user if any of the at least two unique identifiers are the same. According to aspects of the embodiments, the output is audible, visual, or a combination of audible and visual.

According to aspects of the embodiments, a computer is provided for identifying repeat scanning of the same unique identifiers, comprising: a computer is adapted to receive at least two unique identifiers from at least two containers containing an injectable medicament for a modularly assembled medicament, the computer further adapted to check if any of the at least two unique identifiers are different, the computer further adapted to send an output alerting a user if any of the at least two unique identifiers are different.

According to aspects of the embodiments, a processor is provided for identifying duplicate scanning of the same unique identifiers, comprising a processor adapted to receive at least two unique identifiers from at least two containers containing an injectable medicament for a modularly assembled medicament, the processor further adapted to check if any of the at least two unique identifiers are different, the processor further adapted to send an output alerting a user if any of the at least two unique identifiers are different.

According to aspects of the embodiments, software is provided for identifying duplicate scanning of the same unique identifiers, comprising software adapted to receive at least two unique identifiers from at least two containers containing an intracorporeally administered medicament for a modularly assembled medicament, the software further adapted to compare if any of the at least two unique identifiers are different, the software further adapted to send an output alerting a user if any of the at least two unique identifiers are different. According to aspects of the embodiments, the software further configured to send the output to a storage element. According to aspects of the embodiments, the output containing unique identifier information.

According to aspects of the embodiments, a processor is provided for identifying repeat scanning of the same identifiers, comprising: a processor and/or a computer is adapted to receive at least two unique identifiers from at least two containers containing an injectable medicament, the processor and/or computer further adapted to check if any of the at least two unique identifiers are the same, the processor and/or computer further adapted to send an output to a patient specific identifier, to a patient specific profile, to a patient specific medication profile, to a patient specific medication list, to a patient specific medication administration record, to a patient specific storage element, to a patient specific memory element, to a prescription, to an electronic prescription, to a digital prescription, and combinations thereof. According to aspects of the embodiments, the output is linked to a patient specific identifier and/or a patient specific screen/display, to a patient specific profile, to a patient specific medication profile, to a patient specific medication list, to a patient specific medication administration record, to a patient specific storage element, to a patient specific memory element and combinations thereof. According to aspects of the embodiments, the processor and/or computer further comprises a storage element. According to aspects of the embodiments, the link is an electronic link.

According to aspects of the embodiments, a processing device is provided for identifying repeat scanning of the same identifiers, comprising: a processing device adapted to receive at least two unique identifiers from at least two containers containing an injectable medicament, the processing device further adapted to check if any of the at least two unique identifiers are different, the processing device further adapted to send an output to a patient specific identifier, to a patient specific profile, to a patient specific medication profile to a patient specific medication profile, to a patient specific medication administration record, to a patient specific storage element, to a patient specific memory element and combinations thereof. According to aspects of the embodiments, the output is linked to a patient specific identifier and/or a patient specific display, to a patient specific profile, to a patient specific medication profile, to a patient specific medication list, to a patient specific medication administration record, to a patient specific storage element, to a patient specific memory element and combinations thereof. According to aspects of the embodiments, the processing device comprises a processor. According to aspects of the embodiments, the processing is a computer. According to aspects of the embodiments, the processing device further comprises a storage device and/or a storage element. According to aspects of the embodiments, the link is an electronic link.

According to aspects of the embodiments, a method is provided for determining if the same container has been scanned at least twice, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; and determining if the same unique identifier has been scanned more than one time. According to aspects of the embodiments, the method further comprising providing a processing device configured to compare the received unique identifiers from the containers of the modularly assembled medicament. According to aspects of the embodiments, the processing device is a computer. According to aspects of the embodiments, the method is a computer-implemented method.

According to aspects of the embodiments, a method is provided for determining if the same container has been scanned at least twice, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; and double-checking if the same unique identifier has been scanned more than one time. According to aspects of the embodiments, the method further comprising providing a processing device configured to compare the received unique identifiers from the containers of the modularly assembled medicament. According to aspects of the embodiments, the processing device is a computer. According to aspects of the embodiments, the method is a computer-implemented method.

According to aspects of the embodiments, a method is provided for determining if the same container has been scanned at least twice, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; and cross-checking the scanned unique identifiers against each other to determine if the same unique identifier has been scanned more than one time. According to aspects of the embodiments, the method further comprising providing a processing device configured to compare the received unique identifiers from the containers of the modularly assembled medicament. According to aspects of the embodiments, the processing device is a computer. According to aspects of the embodiments, the method is a computer-implemented method.

According to aspects of the embodiments, a method is provided for determining if a container has been scanned at least twice, the method comprising: providing a plurality of containers, the containers containing a beneficial substance for a modularly assembled medicament, the containers having unique identifiers, the unique identifiers adapted to be scanned by a scanning device; providing a scanning device adapted to scan the unique identifiers; scanning the plurality of the containers with the scanning device; and comparing the scanned unique identifiers to determine if the same unique identifier has been scanned more than one time. According to aspects of the embodiments, the method further comprising providing a processing device configured to compare the received unique identifiers from the containers of the modularly assembled medicament. According to aspects of the embodiments, the processing device is a computer. According to aspects of the embodiments, the method is a computer-implemented method.

According to aspects of the embodiments, a method is provided for determining if the same container has been scanned at least twice and providing an output, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; cross-checking the scanned unique identifiers against each other to determine if the same unique identifier has been scanned more than one time; and providing an output, the output alerting a user if it is safe to proceed with administering the contents of the plurality of the containers or if it is not safe to proceed with administering the contents of the plurality of the containers. According to aspects of the embodiments, cross-checking is performed by a processor. According to aspects of the embodiments, producing/providing the output is performed by a processor. According to aspects of the embodiments, cross-checking is performed by a computer. According to aspects of the embodiments, producing/providing the output is performed by a computer. According to aspects of the embodiments, cross-checking is performed by software. According to aspects of the embodiments, producing/providing the output is performed by software. According to aspects of the embodiments, the method is a computer-implemented method.

According to aspects of the embodiments, a method is provided for determining if the same container has been scanned at least twice and providing an output, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; cross-checking the scanned unique identifiers against each other to determine if the same unique identifier has been scanned more than one time; providing an output, the output alerting a user if it is safe to proceed with administering the contents of the plurality of the containers or if it is not safe to proceed with administering the contents of the plurality of the containers; and storing the output in and/or on a memory element. According to aspects of the embodiments, the method is a computer-implemented method. According to aspects of the embodiments, the method further comprising providing a processing device configured to compare the received unique identifiers from the containers of the modularly assembled medicament. According to aspects of the embodiments, the processing device is a computer.

According to aspects of the embodiments, a computer-implemented method is provided for determining if the same container has been scanned at least twice and providing an output, the method comprising: providing a plurality of containers, wherein the containers have a unique identifier adapted to be scanned by a scanning device; providing a scanning device adapted to scan the unique identifiers on the plurality of the containers and deliver the scanned information to a computer; providing a computer; scanning the plurality of the containers with the scanning device and sending the scanned information to the computer;

cross-checking the scanned unique identifiers against each other to determine if the same unique identifier has been scanned more than one time; generating/providing an output, the output alerting a user if it is safe to proceed with administering the contents of the plurality of the containers or if it is not safe to proceed with administering the contents of the plurality of the containers; and storing the output in and/or on a patient specific memory element. According to aspects of the embodiments, cross-checking the scanned unique identifiers is performed by the computer. According to aspects of the embodiments, generating/providing an output is performed by a computer. According to aspects of the embodiments, storing the output is performed by a computer, by a server, or in the cloud.

According to aspects of the embodiments, a method is provided for determining if the same container has been scanned at least twice and providing an output, the method comprising: providing a plurality of containers, wherein the containers have a unique identifier adapted to be scanned by a scanning device; providing a scanning device adapted to scan the unique identifiers on the plurality of the containers; scanning the plurality of the containers with the scanning device; cross-checking the scanned unique identifiers against each other to determine if the same unique identifier has been scanned more than one time; providing an output, the output alerting a user if it is safe to proceed with administering the contents of the plurality of the containers or if it is not safe to proceed with administering the contents of the plurality of the containers; and linking the output to a patient specific data element. According to aspects of the embodiments, the method is a computer-implemented method. According to aspects of the embodiments, the method further comprising providing a processing device configured to compare the received unique identifiers from the containers of the modularly assembled medicament. According to aspects of the embodiments, the processing device is a computer.

According to aspects of the embodiments, the patient specific data element is a patient profile. According to aspects of the embodiments, the patient specific data element is patient specific profile. According to aspects of the embodiments, the patient specific data element is a medication administration record. According to aspects of the embodiments, the patient specific data element is a patient specific medication administration record. According to aspects of the embodiments, the patient specific data element is a medication list. According to aspects of the embodiments, the patient specific data element is a patient specific medication list. According to aspects of the embodiments, the patient specific data element is a patient identifier selected from the group a name, a medical record number, a patient identification number, date of birth and combinations thereof.

According to aspects of the embodiments, a software program is adapted to round a dose of a medicament. According to aspects of the embodiments, the medicament is an injectable medicament. According to aspects of the embodiments, the medicament is an intracorporeally administered medicament. According to aspects of the embodiments, a processor is adapted to round an amount of a beneficial substance. According to aspects of the embodiments, a computer is adapted to round an amount of a beneficial substance. According to aspects of the embodiments, a computer is adapted to round an amount of a medicament. According to aspects of the embodiments, a computer is adapted to round an amount of a beneficial substance to an amount less than about 30% of the prescribed amount. According to aspects of the embodiments, the beneficial substance is an injectable beneficial substance. According to aspects of the embodiments, a computer is adapted to round an amount of a beneficial substance to an amount less than about 20% of the prescribed amount. According to aspects of the embodiments, the beneficial substance is an injectable beneficial substance.

According to aspects of the embodiments, a computer is adapted to round an amount of a beneficial substance to an amount less than about 10% of the prescribed amount. According to aspects of the embodiments, the beneficial substance is an injectable beneficial substance. According to aspects of the embodiments, a computer is adapted to round an amount of a beneficial substance to an amount less than about 5% of the prescribed amount. According to aspects of the embodiments, the beneficial substance is an injectable beneficial substance. According to aspects of the embodiments, the injectable beneficial substance is an intracorporeally administered substance.

According to aspects of the embodiments, a system is provided for determining which containers to dispense for a modular dosage assembly (modular format medicament assembly/modularly administered medicament); comprising: an input characterized in that the input is a final dosage amount; a computer and/or a processor adapted to analyze the final dosage amount, the computer further adapted to select a plurality of containers that will provide the final dosage amount. According to aspects of the embodiments, a system is provided for determining which containers to dispense for a modular dosage assembly (modular format medicament assembly/modularly administered medicament); comprising: an input characterized in that the input is a final dosage amount; a computer and/or a processor adapted to analyze the final dosage amount, the computer and/or processor further adapted to select a plurality of containers from a memory element that will provide the final dosage amount. According to aspects of the embodiments, a system is provided for determining which containers to dispense for a modular dosage assembly (modular format medicament assembly/modularly assembled medicament); comprising: an input characterized in that the input is a final dosage amount; and a computer and/or processor adapted to select a plurality of containers that will provide the final dosage amount. According to aspects of the embodiments, a system is provided for determining which containers to dispense for a modular dosage assembly (modular format medicament assembly/modularly assembled medicament); comprising: an input characterized in that the input is a final dosage amount; a computer and/or processor adapted to select a plurality of container from a memory element that will provide the final dosage amount.

According to aspects of the embodiments, the input is a prescription. According to aspects of the embodiments, the input is an electronic prescription. According to aspects of the embodiments, the input is a digital prescription. According to aspects of the embodiments, the input is a prescription entered by a prescriber. According to aspects of the embodiments, the memory element is characterized in that it is a list of containers. According to aspects of the embodiments, the memory element is characterized in that it stores a list of containers. According to aspects of the embodiments, the memory element is characterized in that is a list of containers containing a beneficial substance. According to aspects of the embodiments, the memory element is characterized in that it stores a list of containers containing a beneficial substance. According to aspects of the embodiments, the beneficial substance is a medicament. According to aspects of the embodiments, the medicament is the same medicament. According to aspects of the embodiments, the list of containers have the same medicament stored in the containers. According to aspects of the embodiments, the list of the containers have different medicaments stored in the containers. According to aspects of the embodiments, the list of containers is the same medicament stored in the same and/or different amounts in the containers.

According to aspects of the embodiments, a system is provided for determining which containers to dispense for a modular dosage assembly; comprising: an input characterized in that the input is a final dosage amount; a computer adapted to analyze the final dosage amount, the computer further adapted to select a plurality of containers from a memory storage element that will provide the final dosage amount. According to aspects of the embodiments, a system is provided for determining which containers to dispense for a modular dosage assembly; comprising: an input characterized in that the input is a final dosage amount; a computer adapted to select a plurality of containers from a memory storage element that will provide the final dosage amount. According to aspects of the embodiments, a system is provided for determining which containers to dispense for a modular dosage assembly (modular format medicament assembly); comprising: an input characterized in that the input is a beneficial substance in a final dosage amount; a computer and/or a processor adapted to receive and analyze the input, the computer and/or the processor further adapted to select a plurality of containers that will provide the beneficial substance in an amount within 30% of the final dosage amount. According to aspects of the embodiments, a system is provided for determining which containers to dispense for a modular dosage assembly (modular format medicament assembly/modularly assembled medicament); comprising: an input characterized in that the input is a beneficial substance in a final dosage amount; a computer and/or processor adapted to receive the input, the computer and/or processor further adapted to select a plurality of containers that will provide the beneficial substance in an amount within 30% of the final dosage amount.

According to aspects of the embodiments, a system is provided for determining which containers to dispense for a modular dosage assembly; comprising: an input characterized in that the input is a beneficial substance in a final dosage amount; a computer adapted to receive and analyze the input, the computer further adapted to select a plurality of containers from a memory element that will provide the beneficial substance in an amount within 30% of the final dosage amount. According to aspects of the embodiments, the computer comprises a processor.

According to aspects of the embodiments, a system is provided for determining which containers to dispense for a modular dosage assembly (modular administered medicament); comprising: an input characterized in that the input is a beneficial substance in a final dosage amount; a processing device adapted to receive and analyze the input, the processing device further adapted to select a plurality of containers from a memory storage element that will provide the beneficial substance in an amount within 30% of the final dosage amount. According to aspects of the embodiments, the processing device is a computer. According to aspects of the embodiments, the processing device is a processor. According to aspects of the embodiments, the processing device is a computer having a processor. According to aspects of the embodiments, the processing device is software. According to aspects of the embodiments, the processing device is a server. According to aspects of the embodiments, the processing device is located in the cloud.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 20% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 20% of the final dosage amount. According to aspects of the embodiments, the processing device is a computer. According to aspects of the embodiments, the processing device is a processor. According to aspects of the embodiments, the processing device is a computer having a processor. According to aspects of the embodiments, the processing device is software. According to aspects of the embodiments, the processing device is a server. According to aspects of the embodiments, the processing device is located in the cloud. According to aspects of the embodiments, the processing device further comprising a storage element.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers from a memory element that will provide the beneficial substance in an amount within 20% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers from a memory element that will provide the beneficial substance in an amount within 20% of the final dosage amount. According to aspects of the embodiments, the processing device is a computer. According to aspects of the embodiments, the processing device is a processor. According to aspects of the embodiments, the processing device is a computer having a processor. According to aspects of the embodiments, the processing device is software. According to aspects of the embodiments, the processing device is a server. According to aspects of the embodiments, the processing device is in the cloud. According to aspects of the embodiments, the processing device further comprising a storage element. According to aspects of the embodiments, the processing device further configured to send a list of the containers and container amounts selected to at least one of a storage element, a patient specific profile, a patient specific medication administration record, a patient specific medication list, a non-patient specific display, and a printer. According to aspects of embodiments, the processing device further configured to generate a unique identifier for each of the plurality of the containers selected by the processing device and to send the unique identifier to a storage element.

According to aspects of the embodiments, a system is provided for determining which containers to dispense for a modular dosage assembly; comprising an input characterized in that the input is a beneficial substance in a final dosage amount; a computer adapted to receive and analyze the input, the computer further adapted to select a plurality of containers from a memory storage element that will provide the beneficial substance in an amount within 20% of the final dosage amount. According to aspects of the embodiments, the processing device further comprising a storage element.

According to aspects of the embodiments, a system for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 10% of the final dosage amount. According to aspects of the embodiments, a system for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 10% of the final dosage amount. According to aspects of the embodiments, the processing device is a computer. According to aspects of the embodiments, the processing device is a processor. According to aspects of the embodiments, the processing device is a computer having a processor. According to aspects of the embodiments, the processing device is a handheld device. According to aspects of the embodiments, the handheld device is a smartphone or a tablet. According to aspects of the embodiments, the computer is a desktop computer or a laptop computer. According to aspects of the embodiments, the processing device is a server. According to aspects of the embodiments, the processing device is in the cloud. According to aspects of the embodiments, the processing device further comprising a storage element.

According to aspects of the embodiments, a system is provided for determining which containers to dispense for a modular dosage assembly; comprising: an input characterized in that the input is a beneficial substance in a final dosage amount; processing device adapted to receive and analyze the input, the processing device further adapted to select a plurality of containers from a memory element that will provide the beneficial substance in an amount within 10% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processor further adapted to select a plurality of containers from a memory element that will provide the beneficial substance in an amount within 10% of the final dosage amount. According to aspects of the embodiments, the processing device is a computer. According to aspects of the embodiments, the processing device is a processor. According to aspects of the embodiments, the processing device is a computer having a processor. According to aspects of the embodiments, the processing device is a handheld device. According aspects of the embodiments, the handheld device is a table, a smartphone, and/or combinations thereof. According to aspects of the embodiments, the processing device is a server. According to aspects of the embodiments, the processing device is in the cloud. According to aspects of the embodiments, the processing device further comprising a storage element.

According to aspects of the embodiments, a system is provided for determining which containers to dispense for a modular dosage assembly; comprising: an input characterized in that the input is a beneficial substance in a final dosage amount; a computer adapted to receive and analyze the input, the computer further adapted to select a plurality of containers from a memory storage element that will provide the beneficial substance in an amount within 10% of the final dosage amount. According to aspects of the embodiments, a system is provided for determining which containers to dispense for a modular dosage assembly; comprising: an input characterized in that the input is a beneficial substance in a final dosage amount; a computer adapted to receive the input, the computer further adapted to select a plurality of containers from a memory storage element that will provide the beneficial substance in an amount within 10% of the final dosage amount.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 5% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 5% of the final dosage amount. According to aspects of the embodiments, the processing device is a computer. According to aspects of the embodiments, the processing device is processor. According to aspects of the embodiments, the processing device is a computer having a processor. According to aspects of the embodiments, the processing device is a server. According to aspects of the embodiments, the processing device is in the cloud.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers from a memory element that will provide the beneficial substance in an amount within 5% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers from a memory element that will provide the beneficial substance in an amount within 5% of the final dosage amount. According to aspects of the embodiments, the processing device is a computer. According to aspects of the embodiments, the processing device is a handheld device. According to aspects of the embodiments, the handheld device is a smartphone or a tablet. According to aspects of the embodiments, the processing device is a processor. According to aspects of the embodiments, the processing device is a server. According to aspects of the embodiments, the processing device is in the cloud.

According to aspects of the embodiments, the input is a prescription. According to aspects of the embodiments, the input is an electronic prescription. According to aspects of the embodiments, the input is a prescription entered by a prescriber. According to aspects of the embodiments, the memory element is characterized in that it is a list of containers. According to aspects of the embodiments, the memory element is characterized in that it is a list of containers containing a beneficial substance. According to aspects of the embodiments, the memory element is characterized in that it is a list of container amounts of a beneficial substance. According to aspects of the embodiments, the container amounts are weight amounts, volume amounts, or weight and volume amounts. According to aspects of the embodiments, the beneficial substance is a medicament. According to aspects of the embodiments, the beneficial substance is a nutritional substance. According to aspects of the embodiments, the medicament is the same medicament. According to aspects of the embodiments, the list of containers have the same medicament stored in the containers. According to aspects of the embodiments, the list of the containers have different medicaments stored in the containers. According to aspects of the embodiments, the list of containers is the same medicament in stored in the same and/or different amounts in the containers.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers from a memory storage element that will provide the beneficial substance in an amount within 5% of the final dosage amount. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers from a memory storage element that will provide the beneficial substance in an amount within 5% of the final dosage amount. According to aspects of the embodiments, the processing device is a computer. According to aspects of the embodiments, the processing device is a processor. According to aspects of the embodiments, the processing device is a smartphone, a table, and/or a handheld device.

According to aspects of the embodiments, the input is a prescription. According to aspects of the embodiments, the input is an electronic prescription. According to aspects of the embodiments, the input is a prescription entered by a prescriber. According to aspects of the embodiments, the memory storage element is characterized in that it is a list of containers. According to aspects of the embodiments, the memory storage element is characterized in that is a list of containers containing a beneficial substance. According to aspects of the embodiments, the beneficial substance is a nutritional substance. According to aspects of the embodiments, the nutritional substance is selected from an amino acid, a protein, a lipid, a fat, a vitamin, a multivitamin, a trace element and combinations thereof. According to aspects of the embodiments, the beneficial substance is a medicament. According to aspects of the embodiments, the medicament is the same medicament. According to aspects of the embodiments, the list of containers have the same medicament stored in the containers. According to aspects of the embodiments, the list of the containers have different medicaments stored in the containers. According to aspects of the embodiments, the list of containers is the same medicament in stored in the same and/or different amounts in the containers. According to aspects of the embodiments, the list of containers is the same medicament stored in about the same amount in the containers.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 30% of the final dosage amount, the processing device further adapted to produce an output data element comprising a list of the plurality of the containers selected. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 30% of the final dosage amount, the processing device further adapted to produce an output data element comprising a list of the plurality of the containers selected. According to aspects of the embodiments, the processing device is a computer. According to aspects of the embodiments, the processing device is a smartphone. According to aspects of the embodiments, the processing device is a handheld device. According to aspects of the embodiments, the processing device is a smartphone with an application (app). According to aspects of the embodiments, the processing device is in the cloud. According to aspects of the embodiments, the processing device is a server.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 20% of the final dosage amount, the processing device further adapted to product an output data element comprising a list of the plurality of the containers selected. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 20% of the final dosage amount, the processing device further adapted to produce an output data element comprising a list of the plurality of the containers selected. According to aspects of the embodiments, the processing device is a computer. According to aspects of the embodiments, the processing device is a processor. According to aspects of the embodiments, the processing device is a smartphone. According to aspects of the embodiments, the processing device is a tablet. According to aspects of the embodiments, the processing device is in the cloud and/or on/in a server.

According to aspects of the embodiments, a system is provided for determining which containers to dispense for a modular dosage assembly; comprising: an input characterized in that the input is a beneficial substance in a final dosage amount; a computer adapted to receive and analyze the input, the computer further adapted to select a plurality of containers that will provide the beneficial substance in an amount within 10% of the final dosage amount, the computer further adapted to produce an output data element comprising a list of the plurality of the containers selected. According to aspects of the embodiments, a system is provided for determining which containers to dispense for a modular dosage assembly; comprising: an input characterized in that the input is a beneficial substance in a final dosage amount; a computer adapted to receive the input, the computer further adapted to select a plurality of containers that will provide the beneficial substance in an amount within 10% of the final dosage amount, the computer further adapted to produce an output data element comprising a list of the plurality of the containers selected.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 30% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record, on a patient specific prescription, and/or combinations thereof. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 30% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record, on a patient specific prescription, and/or combinations thereof. According to aspects of the embodiments, the processing device is a computer. According to aspects of the embodiments, the processing device is a processor. According to aspects of the embodiments, the processing device is a computer having a processor.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 20% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record, displaying the output on a non-patient specific display, and/or combinations thereof. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 20% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record and/or combinations thereof. According to aspects of the embodiments, the processing device is a computer. According to aspects of the embodiments, the processing device is a processor. According to aspects of the embodiments, the processing device is a computer having a processor.

According to aspects of the embodiments, a system is provided for determining which containers to dispense for a modular dosage assembly; comprising: an input characterized in that the input is a beneficial substance in a final dosage amount; a processing device adapted to receive and analyze the input, the processing device further adapted to select a plurality of containers that will provide the beneficial substance in an amount within 10% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record and/or combinations thereof. According to aspects of the embodiments, a system is provided for determining which containers to dispense for a modular dosage assembly; comprising: an input characterized in that the input is a beneficial substance in a final dosage amount; a processing device adapted to receive the input, the processing device further adapted to select a plurality of containers that will provide the beneficial substance in an amount within 10% of the final dosage amount, the processing device further adapted to produce an output data element comprising a list of the plurality of the containers selected; and linking the output data element to a patient specific identifier and/or displaying the output data element on a patient specific profile, on a patient specific medication list, on a patient specific medication administration record and/or combinations thereof. According to aspects of the embodiments, the processing device is a computer. According to aspects of the embodiments, the processing device is a processor. According to aspects of the embodiments, the processing device is a computer having a processor. According to aspects of the embodiments, the processing device is in the cloud. According to aspects of the embodiments, the processing device is in and/or on a server.

According to aspects of the embodiments, a processing device is adapted to select a plurality of containers from a list of containers. According to aspects of the embodiments, the processing device is adapted to select the smallest number of containers to provide the exact final dosage amount prescribed. According to aspects of the embodiments, the processing device is adapted to select the lowest number of containers to provide the exact final dosage amount prescribed. According to aspects of the embodiments, the processing device is adapted to select the smallest number of containers to provide a final dosage amount within 30% of the prescribed dosage amount. According to aspects of the embodiments, the processing device is adapted to select the lowest number of containers to provide a final dosage amount within 30% of the prescribed dosage amount. According to aspects of the embodiments, the processing device is adapted to select the smallest number of containers to provide a final dosage amount within 20% of the prescribed dosage amount. According to aspects of the embodiments, the processing device is adapted to select the lowest number of containers to provide a final dosage amount within 20% of the prescribed dosage amount. According to aspects of the embodiments, the processing device is adapted to select the smallest number of containers to provide a final dosage amount within 10% of the prescribed dosage amount. According to aspects of the embodiments, the processing device is adapted to select the lowest number of containers to provide a final dosage amount within 10% of the prescribed dosage amount. According to aspects of the embodiments, the processing device is adapted to select the smallest number of containers to provide a final dosage amount within 5% of the prescribed dosage amount. According to aspects of the embodiments, the processing device is adapted to select the lowest number of containers to provide a final dosage amount within 3% of the prescribed dosage amount. According to aspects of the embodiments, the processing device is a computer. According to aspects of the embodiments, the processing device is a processor. According to aspects of the embodiments, the processing device is software. According to aspects of the embodiments, the processing device is a computer and/or handheld device having software. According to aspects of the embodiments, the processing device is a server. According to aspects of the embodiments, the processing device is in the cloud.

According to aspects of the embodiments, the processor is adapted to optimize the selection of the containers to be selected for the modular dosage assembly (modular format medicament assembly/modular administered medicament). According to aspects of the embodiments, the optimization takes into account the selection of the lowest/smallest number of containers to provide the final dosage amount. According to aspects of the embodiments, the optimization involves selection of the appropriate container amounts to provide a final dosage as close as possible to the prescribed final dosage amount. According to aspects of the embodiments, the optimization involves selection of the appropriate number of containers and the appropriate amounts to provide a final dosage amount as close as possible to the prescribed final dosage amount using the smallest/lowest number of containers.

According to aspects of the embodiments, the containers are commercially packaged and/or commercially prepackaged containers. According to aspects of the embodiments, the optimization involves scanning a list of commercially available containers that are available to be dispensed to a patient/end-user and selecting a plurality of the commercially available containers to provide a final dosage amount within 20% or less of the prescribed final dosage amount. According to aspects of the embodiments, the optimization involves scanning a list of commercially available containers that are available to be dispensed and selecting a plurality of containers to provide a final dosage amount within 10% or less of the prescribed final dosage amount. According to aspects of the embodiments, the optimization involves scanning a list of commercially available containers a pharmacy has in stock and selecting a plurality of containers to provide a final dosage amount within 30% or less of the prescribed final dosage amount. According to aspects of the embodiments, the optimization involves scanning a list of commercially available containers a pharmacy has in stock and selecting a plurality of containers to provide a final dosage amount within 20% or less of the prescribed final dosage amount. According to aspects of the embodiments, the optimization involves scanning a list of commercially available containers a pharmacy has in stock and selecting a plurality of containers to provide a final dosage amount within 10% or less of the prescribed final dosage amount. According to aspects of the embodiments, the optimization involves scanning a list of commercially available containers a pharmacy has in stock and selecting a plurality of containers to provide a final dosage amount within 5% or less of the prescribed final dosage amount. According to aspects of the embodiments, scanning may be searching. According to aspects of the embodiments, the scanning is conducted by a computer, by a processor, by a computer having a processor, by a handheld device, by a smartphone, by software running on a computer, by software running on a smartphone, by software running on a handheld device, by software running on a server, by software, and combinations thereof.

According to an aspect of the embodiments, a method for verifying contents of medicament containers, comprising: providing at least two medicaments, each medicament in a container, each container having a data-containing tag, the data-containing tag comprising information pertaining to an amount and type of the medicament in the container; scanning the data-containing tags on at least two of the medicament containers; and verifying that the type and amount of the medicaments in the at least two containers matches the prescribed type and amount of the medicament.

In certain embodiments, the at least two medicaments are for a modularly assembled medicament.

In certain embodiments, the step of verifying comprises: obtaining prescription information from at least one of a memory element and a storage element; obtaining information from the data-containing tag of the medicament container based on the scanning step of the data-containing tag of the medicament container; and matching the information from the medicament container data-containing tag with the prescription information.

In certain embodiments, the method is a computer-implemented method.

In certain embodiments, the prescribed type and amount of the medicament is for a specific patient.

In certain embodiments, the prescribed type and amount of the medicament is linked to a patient specific identifier.

In certain embodiments, the link is an electronic link.

In certain embodiments, the link is a digital link.

In certain embodiments, the link identifies a patient when a prescribed amount is identified.

In certain embodiments, the link identifies a prescribed amount when a patient is identified.

In certain embodiments, the prescribed amount of the medicament is for a specific patient.

In certain embodiments, the patient specific identifier is selected from a patient's name, a patient's date of birth, a patient's medical record number, a patient's identification number, a patient's social security number, a patient's bed number, a patient's room number, a patient's identification code, and combinations thereof.

In certain embodiments, the step of matching comprises: summing dosage amount information of the at least two medicament containers (summed dosage of vial container medicaments); obtaining a final dosage amount information from prescription list of medicaments; and determining that the summed dosage amount of the at least two medicament containers is about equal or not equal to the final prescribed dosage amount.

In certain embodiments, the summing dosage amount information is addition of dosage amount information.

In certain embodiments, the summing dosage amount information is addition of dosage amounts.

In certain embodiments, the summing dosage amount information is multiplication of dosage amount information, subtraction of dosage amount information, division of dosage amount information, and combinations thereof.

In certain embodiments, the summing dosage amount information is multiplication of dosage amount information, subtraction of dosage amount information, division of dosage amount information, or combinations thereof.

In certain embodiments, the step of matching comprises: summing dosage amount information of the at least two medicament containers (summed dosage of vial container medicaments); summing dosage amount information of the at least two medicaments listed in a patient specific prescription list (summed dosage of prescription list medicaments); and determining that the summed dosage of prescription list medicaments is about equal to the summed dosage of container medicaments.

In certain embodiments, the summing dosage amount information is addition of dosage amount information.

In certain embodiments, the summing dosage amount information is addition of dosage amounts.

In certain embodiments, the summing dosage amount information is multiplication of dosage amount information, subtraction of dosage amount information, division of dosage amount information, and combinations thereof.

In certain embodiments, the summing dosage amount information is multiplication of dosage amount information, subtraction of dosage amount information, division of dosage amount information, and combinations thereof.

In certain embodiments, the patient specific prescription list is a patient specific medication administration record.

In certain embodiments, the patient specific prescription list is a patient specific profile.

In certain embodiments, the patient specific prescription list is a patient specific prescription.

In certain embodiments, the patient specific prescription list is a prescription.

In certain embodiments, the prescription is an electronic prescription.

In certain embodiments, the determining that the summed dosage of prescription list medicaments is equal to the summed dosage of container medicaments.

In certain embodiments, the step of matching comprises: obtaining a list of the dosage amounts from the at least two medicament containers (list of dosages of vial container medicaments); obtaining a list of the dosage amount information of the at least two medicaments listed in a patient specific profile (list of dosage amounts from prescription list of medicaments); and determining that the list of the dosages from the prescription list of medicaments matches the list of dosages of container amounts.

In certain embodiments, the summing dosage amount information is adding of dosage amounts.

In certain embodiments, the summing dosage amount information is adding dosage amount information.

In certain embodiments, the summing dosage amount information is multiplication of dosage amount information, subtraction of dosage amount information, division of dosage amount information, and combinations thereof.

In certain embodiments, the patient specific prescription list is a patient specific medication administration record.

In certain embodiments, the patient specific prescription list is a patient specific profile.

In certain embodiments, the patient specific prescription list is a patient specific prescription.

In certain embodiments, the patient specific prescription list is a prescription.

In certain embodiments, the prescription is an electronic prescription.

In certain embodiments, the determining that the summed dosage of the prescription list medicaments is equal to the summed dosage of container medicament.

In certain embodiments, the step of matching comprises: obtaining a list of dosage amount information of the at least two medicament containers (list of dosages of vial container medicaments); and comparing the list of dosages of the vial container medicaments to the total final dosage amount.

In certain embodiments, the at least two medicament containers are for a modularly assembled medicament.

In certain embodiments, the at least two medicament containers contain an intracorporeally administered medicament intended for a modular assembled medicament.

In certain embodiments, the total final dosage amount is on a prescription.

In certain embodiments, the total final dosage amount is linked to a prescription.

In certain embodiments, the total final dosage amount is on an electronic prescription.

In certain embodiments, the total final dosage amount is digitally linked to a prescription.

In certain embodiments, the total final dosage amount is displayed on a patient specific profile, on a patient specific medication list, on a patient specific medication administration record, and combinations thereof.

In certain embodiments, the step of matching comprising: obtaining a list of dosage amount information of the at least two medicament containers (list of dosages of vial container medicaments); summing dosage amount information of the at least two medicaments listed in a patient specific profile (summed dosage of prescription list medicaments); and determining that the summed dosage of prescription list medicaments is about equal to the list of dosages of the vial container medicaments.

In certain embodiments, the at least two medicament contains are for a modularly assembled medicament.

In certain embodiments, the at least two medicament containers contain an intracorporeally administered medicament intended for a modular assembled medicament.

In certain embodiments, the total final dosage amount is on a prescription.

In certain embodiments, the total final dosage amount is linked to a prescription.

In certain embodiments, the matching is comparing.

In certain embodiments, the method further comprising issuing a first alert if a no match condition exists.

In certain embodiments, the method further comprising issuing a second alert if a match condition exists.

In certain embodiments, the method is a computer-implemented method.

In certain embodiments, the method is a processor-implemented method.

In certain embodiments, the method is a software-implemented method.

In certain embodiments, the data-containing tag further comprising a unique identifier.

In certain embodiments, the unique identifier characterized in that it differentiates any two containers from each other.

In certain embodiments, the data-containing tag further comprising a unique identification code.

In certain embodiments, the data-containing tag further comprising a unique identifier, the unique identifier characterized in that it differentiates any two containers from each other thus preventing duplicate (double) scanning of the same container.

In certain embodiments, the data-containing tag further comprising a unique identifier, the unique identifier characterized in that it differentiates any two containers labeled as containing the same medicament in the same amount from each other thus preventing duplicate (double) scanning of the same container.

In another aspect of the embodiments, a method is provided for verifying contents of medicament containers, comprising: providing at least two medicaments, each medicament in a container, each container having a data-containing tag, the data-containing tag comprising information pertaining to an amount and type of the medicament in the container, the data containing tag further comprising a unique identifier; scanning the data-containing tags on at least two of the medicament containers; and verifying that the type and amount of the medicaments in the at least two containers matches the prescribed type and amount of the medicament, and further determining that the unique identifier on each of the containers is the same or is different.

In certain embodiments, the method is a computer-implemented method.

In certain embodiments, the method is a software implemented method.

In certain embodiments, the method is a processor implemented method.

In certain embodiments, the step of verifying comprises: obtaining prescription information from at least one of a memory element and a storage element; obtaining information from the data-containing tags of the at least two medicament containers; matching the information from the medicament container data-containing tags with the prescription information; and matching the unique identifier information from the scanned vials to determine if a duplicate (double) scan occurred of the same container.

In certain embodiments, the step of verifying is a computer-implemented method.

In certain embodiments, the step of verifying is a software-implemented method.

In certain embodiments, the step of verifying is a processor-implemented method.

In certain embodiments, the prescribed type and amount of the medicament is for a specific patient.

In certain embodiments, the prescribed type and amount of the medicament is linked to a patient specific identifier.

In certain embodiments, the prescribed type and amount of the medicament is linked to a specific patient.

In certain embodiments, the patient specific identifier is at least one of a medical record number, a date of birth, a patient's name, a patient's social security number, a patient's identification code, a patient's room number, a patient's bed number, and a patient's social security number.

In certain embodiments, the step of matching comprises: summing dosage amount information of the at least two medicament containers (summed dosage of vial container medicaments); obtaining a final dosage amount information from prescription list of medicaments; and determining that the summed dosage amount of the at least two medicament containers is equal or not equal to the final prescribed dosage amount.

In certain embodiments, the summing dosage involves adding dosage amount information of the at least two medicament containers, subtracting dosage amount information of the at least two medicament containers, multiplying dosage amount information of the at least two medicament containers, and/or dividing dosage amount information of the at least two medicament containers.

In certain embodiments, the step of matching comprises: summing dosage amount information of the at least two medicament containers (summed dosage of vial container medicaments); and summing dosage amount information of the at least two medicaments listed in a patient specific prescription list (summed dosage of prescription list medicament); and determining that the summed dosage of prescription list medicaments is equal to the summed dosage of vial container medicaments.

In certain embodiments, the summing dosage involves adding dosage amount information of the at least two medicament containers, subtracting dosage amount information of the at least two medicament containers, multiplying dosage amount information of the at least two medicament containers, and/or dividing dosage amount information of the at least two medicament containers.

In certain embodiments, the step of matching comprises: obtaining a list of the dosage amounts from the at least two medicament containers (list of dosages of vial container medicaments); obtaining a list of the dosage amount information of the at least two medicaments listed in a patient specific profile (list of dosage amounts from prescription list of medicaments); and determining that the list of dosages from the prescription list of medicaments matches the list of dosages of vial container medicaments.

In certain embodiments, the determining that the list of dosages form the prescription list of medicaments matches the list of dosages of vial container medicaments involves cross-checking the list of dosages from the prescription list of medicaments against the list of dosages of vial container medicaments.

In certain embodiments, the determining that the list of dosages form the prescription list of medicaments matches the list of dosages of vial container medicaments involves comparing the list of dosages from the prescription list of medicaments against the list of dosages of vial container medicaments.

In certain embodiments, the step of matching comprises: obtaining a list of dosage amount information of the at least two medicament containers (list of dosages of vial container medicaments); obtaining a final total dosage amount of the at least two medicaments listed in a patient specific profile (total final dosage amount); and comparing the list of dosages of the vial container medicaments to the final dosage amount.

In certain embodiments, the step of matching comprises: obtaining a list of dosage amount information of the at least two medicament containers (list of dosages of vial container medicaments); summing dosage amount information of the at least two medicaments listed in a patient specific profile (summed dosage of prescription list medicaments); and determining that the summed dosage of prescription list medicaments is equal to the list of dosages of vial container medicaments.

In certain embodiments, the summing involves addition, subtraction, multiplication, division, and combinations thereof.

In certain embodiments, the summing involves addition, subtraction, multiplication, division, or combinations thereof.

In certain embodiments, the method further comprising issuing a first alert if a no match condition exits.

In certain embodiments, the method further comprising issuing a second alert if a match condition exists.

In certain embodiments, the method is a computer-implemented method.

In certain embodiments, the method is a software-implemented method.

In yet other aspects of embodiments, a method is provided for verifying contents of medicament containers, comprising: providing at least two medicaments, each medicament in a container, each container having a data-containing tag, the data-containing tag comprising a unique identifier; scanning the data containing tags on at least two of the medicament containers; and determining that the unique identifier on each of the containers is the same or is different.

In certain embodiments, the method is a computer-implemented method.

In certain embodiments, the step of determining that the unique identifiers are the same or different comprises: obtaining information form the data-containing tag of the at least two medicament containers based on the scanning step of the data-containing tag of the medicament container; matching the unique identifier information from the scanned data-containing tags to determining if any of the unique identifiers are the same.

In certain embodiments, the matching the unique identifier information comprises comparing if any two unique identifiers are identical.

In certain embodiments, the method further comprising verifying that each unique identifier of the at least two medicaments of the containers is unique and not repeated.

In certain embodiments, the method further comprising an alert if at least two unique identifiers are the same.

In certain embodiments, the method further comprising an alert if at least two of the unique identifiers are the same, the alert comprising at least one of a visually displayed alert and an audible alert.

In certain embodiments, the method further comprising an alert if at least two unique identifiers are different.

In certain embodiments, the method further comprising an alert if at least two unique identifiers are different, the alert comprising at least one of a visually displayed alert and an audible alert.

In certain embodiments, the method is a computer-implemented method.

In certain embodiments, the method is a software-implemented method.

In yet another aspect of the embodiments, a system is provided for verifying contents of a plurality of medicament containers, comprising: a data processing device adapted to receive prescription information, the prescription information further comprises a dosage amount and a type of a medicament for each of the two or more medicaments; a medicament container adapted to have attached to it the two or more medicaments, each in one or more respective vial containers; and a scanner adapted to scan data-containing tags on each of the respective vial containers, wherein data encoded in the data-containing tag comprises information pertaining to an amount and type of the medicament in the respective vial container, the scanner further adapted to scan a data-containing tag attached to the medicament container, wherein the data processing device is further adapted to verify that the medicaments in the attached vial containers matches prescribed medicament information for a patient.

In certain embodiments, the data-containing tags further comprise a unique identifier for each respective vial container.

In certain embodiments, each vial container has a unique identifier.

In certain embodiments, the data processing device is further adapted to determine if at least any two unique identifiers obtained from the data-containing tags are the same or are different.

In certain embodiments, the data processing device is further adapted to determine if at least any two unique identifiers obtained from the vial containers are the same or are different.

In certain embodiments, the data processing device is configured to cross-check and/or match if any two unique identifiers are identical.

In certain embodiments, the data processing device is configured to compare a plurality of unique identifiers against each other and to determine if any of the plurality of the unique identifiers are identical.

In certain embodiments, the data processing device is further configured to produce an output alerting a user if a duplicate scan of the same vial container has occurred.

In certain embodiments, the data processing device is further configured to produce an output alerting a user if any of the unique identifiers are identical.

In certain embodiments, the data processing device is further configured to produce an output alerting a user if any of the unique identifiers are not identical.

In certain embodiments, the data processing device is further adapted to verify that each serial number of the two or more medicaments of the vial containers is unique and not repeated.

In certain embodiments, the data processing device is further adapted to issue an alert if two or more unique identifiers of the two or more medicaments of the vial containers is the same.

In certain embodiments, the data processing device is further adapted to issue a first alert if a no match condition exists (unique identifiers are different).

In certain embodiments, the data processing device is further adapted to issue a second alert if a match condition exists (at least two of the unique identifiers are the same).

In certain embodiments, the data processing device is a computer.

In certain embodiments, the data processing device is a handheld device.

In certain embodiments, the data processing device has a processor.

In certain embodiments, the data processing device is a server.

In certain embodiments, the data processing device is in the cloud.

In certain embodiments, the computer has a processor.

In certain embodiments, the handheld device is a smartphone or a tablet.

In certain embodiments, the data processing device further comprises a storage device.

In certain embodiments of the system, the step of matching comprises: summing dosage amount information of the at least two medicament containers (summed dosage of vial container medicaments); obtaining a final dosage amount information from prescription list of medicaments; and determining that the summed dosage amount of the at least two medicament containers is equal, not equal, or about equal to the final prescribed dosage amount.

In certain embodiments, the summing of the dosage amount information involves at least one of steps of addition, subtraction, multiplication, and division.

In certain embodiments, the prescription list of medicaments is obtained from at least one of a memory element and a storage element.

In certain embodiments, the prescription list of medicaments is obtained from at least one of a patient profile, a patient's medication administration record, and a patient medication list.

In certain embodiments of the system, the step of matching comprises: obtaining a list of the dosage amounts from the at least two medicament containers (list of dosages of vial container medicaments); obtaining a list of the dosage amount information of the at least two medicaments listed in a patient specific profile (list of dosage amounts from a prescription list of medicaments); and determining that the list of dosages from the prescription list of medicaments matches the list of dosages of vial container medicaments.

In certain embodiments, the prescription list of medicaments is obtained from at least one of a memory element and a storage element.

In certain embodiments, the prescription list of medicaments is obtained from at least one of a patient profile, a patient's medication administration record, and a patient medication list.

In certain embodiments of the system, the step of matching comprises: obtaining a list of dosage amount information of the at least two medicament containers (list of dosages of vial container medicaments); obtaining a final total dosage amount of the at least two medicaments listed in a patient specific profile (total final dosage amount); and comparing the list of dosages of the vial container medicaments to the total final dosage amount.

In certain embodiments of the system, the step of matching comprises: obtaining a list of dosage amount information of the at least two medicament containers (list of dosages of vial container medicaments); summing dosage amount information of the at least two medicaments listed in a patient specific profile (summed dosage of prescription list medicaments); and determining that the summed dosage of prescription list medicaments is equal or not equal to the list of dosages of vial container medicaments.

In certain embodiments, summing dosage amount information involves at least one of addition, subtraction, multiplication, and division.

In certain embodiments, the patient specific profile may be obtained from at least one of a memory element and a storage element.

In certain embodiments, the patient specific profile may be at least on of a medication administration record, a patient specific medication list, and a patient profile.

In another aspect of the invention, a system for verifying contents of a medicament container is provided comprising: a medical data storage and processing device adapted to receive prescription information for a patient and store the prescription information, the prescription information comprising two or more medicament vial container amounts of the same medicament for the patient: a medicament container adapted to have attached to it the two or more medicaments, each in one or more respective vial containers; and a scanner adapted to scan data-containing tags on each of the respective vial containers, wherein data encoded in the data-containing tag comprises information pertaining to an amount and type of medicament in the respective vial container and a unique serial code of each respective vial container, the scanner further adapted to scan a data-containing tag attached to the medicament container, wherein data encoded in the data-containing tag of the medicament container comprises medicament amount and unique serial code information, and wherein the medical data storage and processing device is further adapted to verify that the medicaments in the attached vial containers matches the prescribed medicament information for the patient.

In certain embodiments, the unique serial code is a unique identifier.

In certain embodiments, the unique serial code is a unique identification number.

In yet another aspect, a system is provided for preventing duplicate scanning of the same container for modularly assembled medicaments, comprising: a plurality of containers containing a beneficial substance, the plurality of the containers having unique identifiers, the unique identifiers configured to be scanned by an acquisition device, the unique identifiers characterized in that they uniquely identify each container of the plurality of the containers as being distinctly different from the other containers of the plurality of the containers; a processing device configured to receive the unique identifier information from the acquisition device, the processing device further configured to compare the received unique identifier information from the plurality of the containers, the processing device further configured to determine if any of the unique identifiers are the same or not.

In certain embodiments, each of the unique identifiers is different.

In certain embodiments, each of the containers is manufactured by the same manufacturer and each of the containers having a uniquely different unique identifier.

In certain embodiments, at least two of the containers is manufactured by a different manufacturer and each of the containers having a uniquely different unique identifier.

In certain embodiments, the unique identifiers are configured to be scanned by a scanner.

In certain embodiments, the acquisition device is a scanner.

In certain embodiments, the acquisition device is configured to scan the unique identifiers.

In certain embodiments, the unique identifiers are in barcode form.

In certain embodiments, the unique identifiers are embedded and/or encoded in barcode form on the containers.

In certain embodiments, the processing device is a computer.

In certain embodiments, the processing device is a processor.

In certain embodiments, the processing device is a handheld device.

In certain embodiments, the processing device is a smartphone or a tablet.

In certain embodiments, the processing device is further configured to have a storage element.

In certain embodiments, the processing device is further configured to have a memory element.

In certain embodiments, the processing device is further configured to generate an output.

In certain embodiments, the output alerts a user if a duplicate scan of the same container has occurred.

In certain embodiments, the output alerts a user if a duplicate scan of the same container has not occurred.

In yet another aspect of the embodiments, an apparatus is provided for receiving a plurality of unique identifier information from an acquisition device, the apparatus further configured to compare the received unique identifier information and to determine if any two unique identifiers are the same or are different.

In certain embodiments, the apparatus is further configured to generate an output.

In certain embodiments, the output alerts a user if the apparatus has received at least two identical unique identifiers.

In certain embodiments, the output alerts a user if all of the received unique identifiers are different.

In yet another aspect of the embodiments, an apparatus is provided for identifying duplicate scans of the same container, comprising: software configured to receive a plurality of unique identifier information and to compare the unique identifier information, the software further configured to determine if any of the received unique identifiers are the same or are different.

In yet another aspect of the embodiments, a processing device is provided for selecting a plurality of vial containers containing a beneficial substance, the processing device configured to select the least amount of vial containers that match a prescribed dosage amount as close as possible.

In certain embodiments, the selecting of the plurality of the vial containers includes selecting from a list of known available vial containers.

In certain embodiments, the processing device is further configured to generate a list of the selected vial containers.

In certain embodiments, the processing device is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processing device is further configured to store the generated list of selected vial containers on a non-patient specific display.

In certain embodiments, the processing device is further configured to store the generated list of selected vial containers on at least one of a storage element and a memory element.

In certain embodiments, the processing device is further configured to store the generated list of selected vial containers on at least one of the prescription and patient specific prescription.

In certain embodiments, the processing device is further configured to store the generated list of selected vial containers in at least one of the prescription and patient specific prescription.

In certain embodiments, the processing device is further configured to link the generated list of selected vial containers to at least one of a prescription and a patient specific prescription.

In certain embodiments, the processing device is further configured to link the generated list of the selected vial containers to at least one of a prescription and a patient specific prescription.

In certain embodiments, the processing device is further configured to link the generated list of the selected vial containers to the prescription.

In certain embodiments, the processing device is further configured to link the generated list of the selected vial containers to the patient specific prescription.

In certain embodiments, the processing device is further configured to link the generated list of the selected vial containers to at least one of a patient specific display, a patient specific profile, a patient specific medication list, a patient specific medication administration record MAR, a non-patient specific display, and a non-patient specific medication list. In certain embodiments, the processing device is a computer.

In certain embodiments, the processing device is selected from at least one of a handheld device, a smartphone, and a server.

In certain embodiments, the processing device is a computer.

In yet another aspect of the embodiments, a processing device is provided for selecting a plurality of vial containers containing a beneficial substance, the processing device configured to select the amount of vial containers that match a prescribed dosage amount as close as possible.

In certain embodiments, the selecting of the plurality of the vial containers includes selecting from a list of known available vial containers.

In certain embodiments, the processing device is further configured to generate a list of the selected vial containers.

In certain embodiments, the processing device is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processing device is further configured to store the generated list of selected vial containers on a non-patient specific display.

In certain embodiments, the processing device is further configured to store the generated list of selected vial containers on at least one of a storage element and a memory element.

In certain embodiments, the processing device is further configured to store the generated list of selected vial containers on at least one of the prescription and patient specific prescription.

In certain embodiments, the processing device is further configured to store the generated list of selected vial containers in at least one of the prescription and patient specific prescription.

In certain embodiments, the processing device is further configured to link the generated list of selected vial containers to at least one of a prescription and a patient specific prescription.

In certain embodiments, the processing device is further configured to link the generated list of the selected vial containers to at least one of a prescription and a patient specific prescription.

In certain embodiments, the processing device is further configured to link the generated list of the selected vial containers to the prescription.

In certain embodiments, the processing device is further configured to link the generated list of the selected vial containers to the patient specific prescription.

In certain embodiments, the processing device is further configured to link the generated list of the selected vial containers to at least one of a patient specific display, a patient specific profile, a patient specific medication list, a patient specific medication administration record MAR, a non-patient specific display, and a non-patient specific medication list.

In certain embodiments, the processing device is a computer.

In certain embodiments, the processing device is selected from at least one of a handheld device, a smartphone, and a server.

In yet another aspect of the embodiments, a processing device is provided for selecting a plurality of vial containers containing a beneficial substance, the processing device configured to select and match a final dosage amount within a predetermined range of error.

In certain embodiments, the predetermined range of error is less than about 10% of a prescribed amount.

In certain embodiments, the predetermined range of error is less than 10% of a prescribed amount.

In certain embodiments, the selecting of the plurality of the vial containers includes selecting from a list of known available vial containers.

In certain embodiments, the processing device is further configured to generate a list of the selected vial containers.

In certain embodiments, the processing device is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processing device is further configured to store the generated list of selected vial containers on a non-patient specific display.

In certain embodiments, the processing device is further configured to store the generated list of selected vial containers on at least one of a storage element and a memory element.

In certain embodiments, the processing device is further configured to store the generated list of selected vial containers on at least one of the prescription and patient specific prescription.

In certain embodiments, the processing device is further configured to store the generated list of selected vial containers in at least one of the prescription and patient specific prescription.

In certain embodiments, the processing device is further configured to link the generated list of selected vial containers to at least one of a prescription and a patient specific prescription.

In certain embodiments, the processing device is further configured to link the generated list of the selected vial containers to at least one of a prescription and a patient specific prescription.

In certain embodiments, the processing device is further configured to link the generated list of the selected vial containers to the prescription.

In certain embodiments, the processing device is further configured to link the generated list of the selected vial containers to the patient specific prescription.

In certain embodiments, the processing device is further configured to link the generated list of the selected vial containers to at least one of a patient specific display, a patient specific profile, a patient specific medication list, a patient specific medication administration record MAR, a non-patient specific display, and a non-patient specific medication list.

In certain embodiments, the processing device is a computer.

In certain embodiments, the processing device is selected from at least one of a handheld device, a smartphone, and a server.

In yet another aspect of the embodiments, a processor is provided for selecting a plurality of vial containers containing a beneficial substance, the processor configured to select the least amount of vial containers that match a prescribed dosage amount as close as possible.

In certain embodiments, the selecting of the plurality of the vial containers includes selecting from a list of known available vial containers.

In certain embodiments, the processor is further configured to generate a list of the selected vial containers.

In certain embodiments, the processor is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processor is further configured to store the generated list of selected vial containers on a non-patient specific display.

In certain embodiments, the processor is further configured to store the generated list of selected vial containers on at least one of a storage element and a memory element.

In certain embodiments, the processor is further configured to store the generated list of selected vial containers on at least one of the prescription and patient specific prescription.

In certain embodiments, the processor is further configured to store the generated list of selected vial containers in at least one of the prescription and patient specific prescription.

In certain embodiments, the processor is further configured to link the generated list of selected vial containers to at least one of a prescription and a patient specific prescription.

In certain embodiments, the processor is further configured to link the generated list of the selected vial containers to at least one of a prescription and a patient specific prescription.

In certain embodiments, the processor is further configured to link the generated list of the selected vial containers to the prescription.

In certain embodiments, the processor is further configured to link the generated list of the selected vial containers to the patient specific prescription.

In certain embodiments, the processor is further configured to link the generated list of the selected vial containers to at least one of a patient specific display, a patient specific profile, a patient specific medication list, a patient specific medication administration record MAR, a non-patient specific display, and a non-patient specific medication list. In certain embodiments, the processing device is a computer.

In certain embodiments, the processor is selected from at least one of a handheld device, a smartphone, and a server.

In certain embodiments, the processor is a computer.

In yet another aspect of the embodiments, a processor is provided for selecting a plurality of vial containers containing a beneficial substance, the processor configured to select the amount of vial containers that match a prescribed dosage amount as close as possible.

In certain embodiments, the selecting of the plurality of the vial containers includes selecting from a list of known available vial containers.

In certain embodiments, the processor is further configured to generate a list of the selected vial containers.

In certain embodiments, the processor is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processor is further configured to store the generated list of selected vial containers on a non-patient specific display.

In certain embodiments, the processor is further configured to store the generated list of selected vial containers on at least one of a storage element and a memory element.

In certain embodiments, the processor is further configured to store the generated list of selected vial containers on at least one of the prescription and patient specific prescription.

In certain embodiments, the processor is further configured to store the generated list of selected vial containers in at least one of the prescription and patient specific prescription.

In certain embodiments, the processor is further configured to link the generated list of selected vial containers to at least one of a prescription and a patient specific prescription.

In certain embodiments, the processor is further configured to link the generated list of the selected vial containers to at least one of a prescription and a patient specific prescription.

In certain embodiments, the processor is further configured to link the generated list of the selected vial containers to the prescription.

In certain embodiments, the processor is further configured to link the generated list of the selected vial containers to the patient specific prescription.

In certain embodiments, the processor is further configured to link the generated list of the selected vial containers to at least one of a patient specific display, a patient specific profile, a patient specific medication list, a patient specific medication administration record MAR, a non-patient specific display, and a non-patient specific medication list.

In certain embodiments, the processor is a computer.

In certain embodiments, the processor is selected from at least one of a handheld device, a smartphone, and a server.

In yet another aspect of the embodiments, a processor is provided for selecting a plurality of vial containers containing a beneficial substance, the processing device configured to select and match a final dosage amount within a predetermined range of error.

In certain embodiments, the predetermined range of error is less than about 10% of a prescribed amount.

In certain embodiments, the predetermined range of error is less than 10% of a prescribed amount.

In certain embodiments, the selecting of the plurality of the vial containers includes selecting from a list of known available vial containers.

In certain embodiments, the processor is further configured to generate a list of the selected vial containers.

In certain embodiments, the processor is further configured to display the generated list of selected vial containers on at least one of a patient specific display, a patient specific profile, a patient specific medication list, a medication administration record MAR, a patient specific medication record MAR, a prescription, an electronic prescription, a digital prescription, a patient specific prescription, a patient specific electronic prescription, and a patient specific digital prescription.

In certain embodiments, the processor is further configured to store the generated list of selected vial containers on a non-patient specific display.

In certain embodiments, the processor is further configured to store the generated list of selected vial containers on at least one of a storage element and a memory element.

In certain embodiments, the processor is further configured to store the generated list of selected vial containers on at least one of the prescription and patient specific prescription.

In certain embodiments, the processor is further configured to store the generated list of selected vial containers in at least one of the prescription and patient specific prescription.

In certain embodiments, the processor is further configured to link the generated list of selected vial containers to at least one of a prescription and a patient specific prescription.

In certain embodiments, the processor is further configured to link the generated list of the selected vial containers to at least one of a prescription and a patient specific prescription.

In certain embodiments, the processor is further configured to link the generated list of the selected vial containers to the prescription.

In certain embodiments, the processor is further configured to link the generated list of the selected vial containers to the patient specific prescription.

In certain embodiments, the processor is further configured to link the generated list of the selected vial containers to at least one of a patient specific display, a patient specific profile, a patient specific medication list, a patient specific medication administration record MAR, a non-patient specific display, and a non-patient specific medication list.

In certain embodiments, the beneficial substance is a medicament.

In yet another aspect of the embodiments, a method is provided for selecting a plurality of containers containing a beneficial substance, the method comprising: providing a prescribed final dosage amount of a beneficial substance; and selecting a plurality of containers, the selecting characterized in that the least amount of containers are selected that match the final dosage amount as close as possible.

In certain embodiments, the method is a computer-implemented method.

In certain embodiments, the beneficial substance is a medicament.

In certain embodiments, the beneficial substance is a nutritional substance.

In certain embodiments, the method is a computer-implemented method.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In yet another aspect of the embodiments, a method is provided for selecting a plurality of containers containing a beneficial substance, the method comprising: providing a prescribed final dosage amount of a beneficial substance; and selecting a plurality of containers, the selecting characterized in that the containers are selected that match the final dosage amount as close as possible.

In certain embodiments, the method is a computer-implemented method.

In certain embodiments, the beneficial substance is a medicament.

In certain embodiments, the beneficial substance is a nutritional substance.

In certain embodiments, the method is a computer-implemented method.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In yet another aspect of the embodiments, a method is provided for selecting a plurality of containers containing a beneficial substance, the method comprising: providing a prescribed final dosage amount of a beneficial substance; and selecting a plurality of containers, the selecting characterized in that the containers are selected to match the final dosage amount within a predetermined range of error.

In certain embodiments, the method is a computer-implemented method.

In certain embodiments, the predetermined range of error is a percentage.

In certain embodiments, the predetermined range of error is a dosage amount.

In certain embodiments, the predetermined range of error is a volume amount.

In certain embodiments, the beneficial substance is a medicament.

In certain embodiments, the beneficial substance is a nutritional substance.

In certain embodiments, the method is a computer-implemented method.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In certain embodiments, the software further configured to generate an output alerting a user if a duplicate scan of the same container has occurred or not.

In yet another aspect of the embodiments, a method is provided for selecting a plurality of containers containing a beneficial substance, the method comprising: providing a prescribed list of medicaments; and selecting a plurality of containers, the selecting characterized in that the least amount of containers are selected that match the prescribed list of medicaments as close as possible.

In certain embodiments, the prescribed list of medicaments includes a prescribed list of medicament amounts and the selecting includes selecting the least amount of containers that match the prescribed list of medicament amounts as close as possible.

In certain embodiments, the method is a computer-implemented method.

In certain embodiments, the medicament may be a nutritional substance.

In certain embodiments, the beneficial substance is a nutritional substance.

In certain embodiments, the method is a computer-implemented method.

In certain embodiments, the selecting is further characterized in that the known available containers are selected to match a final dosage amount of the prescribed list of medicaments as close as possible.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In yet another aspect of the embodiments, a method is provided for selecting a plurality of containers containing a beneficial substance, the method comprising: providing a prescribed list of medicaments; and selecting a plurality of containers, the selecting characterized in that known available containers are selected to match the prescribed list of medicaments as close as possible.

In certain embodiments, the method is a computer-implemented method.

In certain embodiments, the beneficial substance is a medicament.

In certain embodiments, the beneficial substance is a nutritional substance.

In certain embodiments, the method is a computer-implemented method.

In certain embodiments, the selecting is further characterized in that the known available containers are selected to match a final dosage amount of the prescribed list of medicaments as close as possible.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In yet another aspect of the embodiments, a method is provided for selecting a plurality of containers containing a beneficial substance, the method comprising: providing a prescribed list of medicaments; and selecting a plurality of containers, the selecting characterized in that the containers are selected to match the prescribed list of medicaments within a predetermined range of error.

In certain embodiments, the method further comprises selecting containers that match the final total dosage of the prescribed list of medicaments within a predetermined range of error.

In certain embodiments, the method is a computer-implemented method.

In certain embodiments, the predetermined range of error is a percentage.

In certain embodiments, the predetermined percentage is less than about 10%.

In certain embodiments, the predetermined range of error is less than 10%.

In certain embodiments, the predetermined range of error is a dosage amount.

In certain embodiments, the predetermined range of error is a volume amount.

In certain embodiments, the beneficial substance is a medicament.

In certain embodiments, the beneficial substance is a nutritional substance.

In certain embodiments, the method is a computer-implemented method.

In certain embodiments, the selecting is further characterized in that the known available containers are selected to match a final dosage amount of the prescribed list of medicaments within a predetermined range of error.

In certain embodiments, the predetermined range of error is a percentage.

In certain embodiments, the predetermined range of error is less than about 10%.

In certain embodiments, the predetermined range of error is less than 10%.

In certain embodiments, the selecting of the plurality of the containers includes selecting from a list of known available containers.

In certain embodiments, the method further comprises generating a list of the selected containers.

In certain embodiments, the method further comprises displaying the generated list of selected containers on at least one of a patient specific display, a patient specific profile, a patient specific medication list, a medication administration record MAR, a patient specific medication record, a prescription, an electronic prescription, a digital prescription, a patient specific prescription, a patient specific electronic prescription, and a patient specific digital prescription.

In certain embodiments, the method further comprising storing the generated list of selected containers.

In certain embodiments, the method further comprising storing the generated list of selected containers on at least one of a storage element and a memory element.

In certain embodiments, the method further comprising storing the generated list of selected containers on a prescription.

In certain embodiments, the method further comprising storing the generated list of selected containers on a patient specific prescription.

In certain embodiments, the method further comprising linking the generated list of selected containers to at least one of a prescription and a patient specific prescription.

In certain embodiments, the method further comprising linking the generated list of selected containers to the prescription.

In certain embodiments, the method further comprising linking the generated list of selected containers to the patient specific prescription.

In certain embodiments, the prescription is an electronic prescription.

In certain embodiments, the prescription is a digital prescription.

In certain embodiments, the patient specific prescription is an electronic prescription.

In certain embodiments, the patient specific prescription is a digital prescription.

In certain embodiments, the method is further configured to link the generated list of selected containers to at least one of a patient specific display, a patient specific profile, a patient specific medication list, a patient specific medication administration record, a non-patient specific display, and a non-patient specific medication list.

In certain embodiments, the method is implemented on a handheld device.

In certain embodiments, the handheld device is a tablet.

In certain embodiments, the handheld device is a smartphone.

In certain embodiments, the method is implemented on a computer.

In certain embodiments, the method is implemented by a server.

In certain embodiments, the method is implemented in the cloud.

In certain embodiments, the software further configured to generate an output alerting a user if a duplicate scan of the same container has occurred or not.

In yet a further aspects of the embodiments, a system is provided for comparing unique identifier information for a modularly administered medicament, comprising: a modularly administered medicament having a plurality of containers, wherein a plurality of the containers have unique identifiers; and a processing device configured to receive the unique identifiers from the modularly administered medicament containers, the processing device further configured to compare the unique identifiers and to determine if a duplicate unique identifier has been received by the processing device.

In certain embodiments, the processing device is a computer.

In certain embodiments, the processing device is a smartphone.

In certain embodiments, the processing device is a tablet.

In certain embodiments, the processing device is a server.

In certain embodiments, the processing device is in the cloud.

In certain embodiments, the processing device further comprises at least one of a storage element and a memory element.

In certain embodiments, the system further comprising an acquisition device configured to scan the containers of the modularly administered medicament and send unique identifier information from the containers to the processing device.

In certain embodiments, the processing device further configured to generate an output alerting a user if a duplicate scan has occurred of the same container or if a duplicate scan of the same container has not occurred.

In certain embodiments, the unique identifier information processed by the processing device is not predetermined.

In certain embodiments, predetermined means not known to the processing device prior to receiving the unique identifier information.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates a conventional medicament administration device/container.

FIG. 2A illustrates a modularly assembled medicament container in which a plurality of containers containing prescribed medicaments are attached/connected to an intravenous (IV) bag (or other container) according to aspects of the embodiments.

FIG. 2B illustrates a modularly assembled medicament container in which a plurality of containers containing substantially similar prescribed medicaments are attached/connected to an intravenous (IV) bag according to aspects of the embodiments.

FIG. 3A illustrates stored data elements and data elements acquired by the scanning device in a list form for several different medicaments according to aspects of the embodiments

FIG. 3B illustrates a stored data element and a plurality of data elements acquired by the scanning device in a list form according to aspects of the embodiments.

FIG. 4 illustrates a flow chart of a method for comparing data elements obtained from two or more containers comprising part of the modularly assembled medicament container/system as shown in FIG. 2A and 2B against a prescription, a medication administration record, and/or patient medication list/patient specific medication list according to aspects of the embodiments.

FIG. 5 illustrates an un-assembled modularly assembled medicament containers/system in which a plurality of containers containing prescribed medicaments can be attached/connected to an intravenous (IV) bag (or other container) according to aspects of the embodiments.

FIG. 6 illustrates a further embodiment of a modularly assembled medicament container/system with three vials attached thereto and multiple destinations of data that can be scanned by a scanner according to aspects of the embodiments.

FIG. 7 illustrates a flow chart of a method for comparing data elements obtained from two or more containers comprising part of the modularly assembled medicament containers/system as shown in FIG. 2A and 2B against a medication administration record, a prescription list, a medication list, and/or a patient specific medication list according to aspects of the embodiments.

FIG. 8 illustrates a flow chart of a method for selecting one or more medicament containers for use with the modularly assembled medicament containers/system shown in FIGS. 2A and 2B according to aspects of the embodiments.

FIG. 9 illustrates a block diagram of a medication administration verification (MAV) system adapted to verify that delivered medicaments match prescribed medicaments according to aspects of the embodiments.

FIG. 10 illustrates a block diagram of components of a personal computer or server suitable for use to implement one or more of the methods 400, 700, and 800 described in FIGS. 4, 7, and 8 according to aspects of the embodiments.

FIG. 11 illustrates a network system within which the medication administration verification system as shown in FIG. 9, and also described in detail in regard to FIGS. 1-10, can be implemented according to aspects of the embodiments.

FIG. 12 illustrates a flow chart of a method for comparing unique identifier data elements obtained from two or more containers comprising part of the modularly assembled medicament container as shown in FIGS. 2A and 2B against each other to eliminate duplicate medicaments according to aspects of the embodiments.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary medical devices/containers/systems/methods for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The embodiments are described more fully hereinafter with reference to the accompanying drawings, in which embodiments of the inventive concept are shown. In the drawings, the size and relative sizes of layers and regions can be exaggerated for clarity. Like numbers refer to like elements throughout. The embodiments can, however, be embodied in many different forms and should not be construed as limited to the embodiments set forth herein. Rather, these embodiments are provided so that this disclosure will be thorough and complete, and will fully convey the scope of the inventive concept to those skilled in the art. The following embodiments are discussed, for simplicity, with regard to the terminology and structure of a medical services environments, such as a hospital, nursing home, doctor's office, walk-in clinic, or home infusion setting.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” or “in an embodiment” in various places throughout the specification is not necessarily referring to the same embodiment. Further, the particular feature, structures, or characteristics can be combined in any suitable manner in one or more embodiments.

Definitions:

“Linked”—as used herein, denotes the interconnection/inter-relationship of two or more items, having or providing something with links, and/or a connecting element or factor, and/or an identifier attached to an element in a system (such as an index term in a database) in order to indicate or permit connection with other similarly identified elements.

“Storage”—a memory or other storage device.

“Processor”—a hardware device programmed with computer program instructions to perform a programmable function. The processor may be a microprocessor. The processor may be a central processing unit.

“Device”—a device can be a cloud based device or portable device such as a smartphone. A device may also be a stationary computer and/or a laptop computer.

“Data entry”—entry from vial/container into software, which may occur by SCANNING TAGS on vials/containers or by MANUAL ENTRY via keyboard by a user.

“Patient Profile”—A display/screen that shows patient specific information such as a patient's date of birth, name, medical record number, identification number, social security number, patient weight/height/body surface area, renal function, liver function, laboratory results, diagnosis, hospital admission information, blood pressure, medication list, etc.

“Patient Specific List” and/or a “Patient Specific Medication List”—linked (electronically linked/connected) to a patient identifier that is a list of medications and is on a MEDICATION ADMINISTRATION RECORD, PATIENT PROFILE, PATIENT MEDICATION LIST—this may also be a list of medications a patient is receiving but may also contain an ELECTRONIC DAY/TIME STAMP RECORD of when nurse/caregiver administered drug to patient.

“SUMMED/SUMMING”—mathematical calculation involving at least one of addition, subtraction, multiplication, and division.

“MATCHES/MATCHING”—correlating and/or comparing.

“VERIFYING”—accurately confirming.

“Data-containing tag”—tag containing information (such as but not limited to identity/name of medication, amount of medication, volume of medication, unique identifier information, etc.) that may be printed on a container, may be attached/affixed to a container, may be embedded on a surface of a container, etc.

“Patient specific identifier”—identifier that includes date of birth, medical record number, patient name, patient social security number, patient bed number, patient room number, patient identification code, and/or anything that specifically identifies a patient.

“Modularly Administered Medicament”—medicament that includes but not limited to a medicament/beneficial substance that is assembled using commercially packaged/prepackaged containers (medication not intermixed/mixed/compounded by hand, pumps, and/or robots). In certain embodiments, medicament may be medicament that is customized/individualized final dosage without intermixing/mixing/compounding. In certain embodiments, medicament may be customized/individualized “assembly” of a medicament using commercially packaged/prepackaged containers without the need to measure and/or intermix/mix/compound the medicament. In certain embodiments, medicament may be any beneficial substance including drugs, nutritional substances, diagnostic substances, etc.

Used throughout the specification are several acronyms, the meanings of which are provided as follows:

3G Third Generation
4G Fourth Generation
AI Artificial Intelligence
App Executable Software Programming Code/Application
AR Augmented Reality
ASIC Application Specific Integrated Circuitry
BIOS Basic Input/Output System
BT BlueTooth
CD Compact Disk
CRT Cathode Ray Tubes
DVD Digital Video Disk
EEPROM Electrically Erasable Programmable Read Only Memory
FPGA Field Programmable Gate Array Structures
GAN Global Area Network
GPS Global Positioning System
HDD Hard Disk Drive
HDMI High Definition Multimedia Interface
ISP Internet Service Provider
IV Intravenous
LCD Liquid Crystal Display
LED Light Emitting Diode Display
LTE Long Term Evolution
MAM Modularly Assembled Medicament (used interchangeably with Modular Format Medicament Assembly)
MAR Medication Administration Record—A list/record of medications a specific patient is on/receiving/prescribed
MAV Medication Administration Verification
MMPL Medication Administration Record Medicament Prescription List
MODEM Modulator-Demodulator
NFC Near Field Communications
PC Personal Computer
PED Personal Electronic Device
POTS Plain Old Telephone Service
RAM Random Access Memory
ROM Read-Only Memory
RW Read/Write
SN Serial Number
UPC Universal Product Code
USB Universal Serial Bus
UVPROM Ultra-violet Erasable Programmable Read Only Memory
VGA Video Graphics Array

The following is a list of the elements of the Figures in numerical order:
100 Medication Dispensing Apparatus
102 Intravenous (IV) Bag/Bottle/Syringe (Bag)
104 Universal Product Code (UPC) Label (Barcode)
106 Infusion Line
200 Modularly Assembled Medicament (MAM) Container
202 Vial
204 Vial Barcode
206 Data Acquisition Device/Scanning Device (Scanner)
302 Medication Administration Record (MAR) Medicament Prescription List (MMPL)
304 Modularly Assembled Medicament Container Drug Type and Amount List (Stored Data Elements)
400 Method for Comparing a Medication Administration Record (MAR) Medicament Prescription List (MMPL) versus MAM Container Drug Type and Amount List (Stored Data Elements)
402-410 Method Steps of Method 400
602 Patient Specific Pharmacy Profile Record 602
604 Patient Specific Medication Administration Record (MAR)
700 Method for Comparing Data Elements Obtained from Two or More Containers Comprising Part of the modularly Assembled Medicament Container Against a Medication Administration Record Prescription List
702-718 Steps for Method 700
800 Method for Selecting One or More Medicament Vials 202 for Use with a Modularly Assembled Medicament Container
802-822 Steps for Method 800
900 Medication Administration Verification (MAV) System
902 Display
904 Computer (Medication Administration Verification (MAV) Computer)
906 Microprocessor
907 Data Bus
908 Memory
910 Medication Administration Verification Program/Application (MAV App)
912 Communications Interface
1001 Shell/Box
1002 Integrated Display/Touch-Screen (laptop/tablet etc.)
1004 Internal Data/Command Bus (Bus)
1006 Processor Internal Memory
1008 Processor(s)
1010 Universal Serial Bus (USB) Port
1011 Ethernet Port
1012 Compact Disk (CD)/Digital Video Disk (DVD) Read/Write (RW) CD/DVD/RW) Drive
1014 Floppy Diskette Drive
1016 Hard Disk Drive (HDD)
1018 Read-Only Memory (ROM)
1020 Random Access Memory (RAM)
1022 Video Graphics Array (VGA) Port or High Definition Multimedia Interface (HDMI)
1024 External Memory Storage Device
1026 External Display/Touch-Screen
1028 Keyboard
1030 Mouse
1032 Processor Board/PC Internal Memory (Internal Memory)
1034 Flash Drive Memory
1036 CD/DVD Diskettes
1038 Floppy Diskettes
1042 Wi-Fi Transceiver
1044 BlueTooth (BT) Transceiver
1046 Near Field Communications (NFC) Transceiver
1048 Third Generation (3G), Fourth Generation (4G), Long Term Evolution (LTE) (3G/4G/LTE) Transceiver
1050 Communications Satellite/Global Positioning System (Satellite) Transceiver Device
1052 Antenna
1054 Internet
1056 Universal Serial Bus (USB) Cable
1058 Ethernet Cable (CAT5)
1060 Scanner/Printer/Fax Machine
1100 Network System
1102 Mobile Device
904 MAV Computer
1106 Internet Service Provider (ISP)
1108 Modulator/Demodulator (Modem)
1110 Wireless Router
1112 Plain Old Telephone Service (POTS) Provider
1114 Cellular Service Provider
1118 Communication Satellites
1120 Cellular Telecommunications Service Tower (Cell Tower)
1124 GPS Station
1126 Satellite Communication Systems Control Station
1128 Global Positioning System (GPS) Satellite

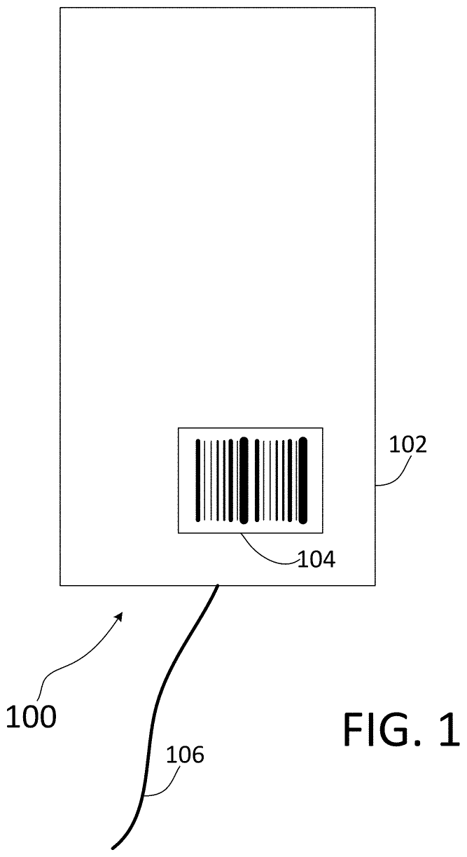

It is important to note that FIGS. 1-12 and corresponding descriptions/discussion below illustrate various aspects of the embodiments referring to a medication administration record MAR. However, it is expressly stated here that the embodiments shown and discussed throughout the herein entire specification are not limited to the MAR. Therefore, when referring to the medication administration record MAR in FIGS. 1-12 and in the corresponding descriptions/discussion (as well as throughout this entire specification) the medication administration record MAR may be replaced with (and/or may be meant to also mean) a patient profile, a patient specific profile, a patient specific medication list, any patient specific display/screen, and combinations thereof.

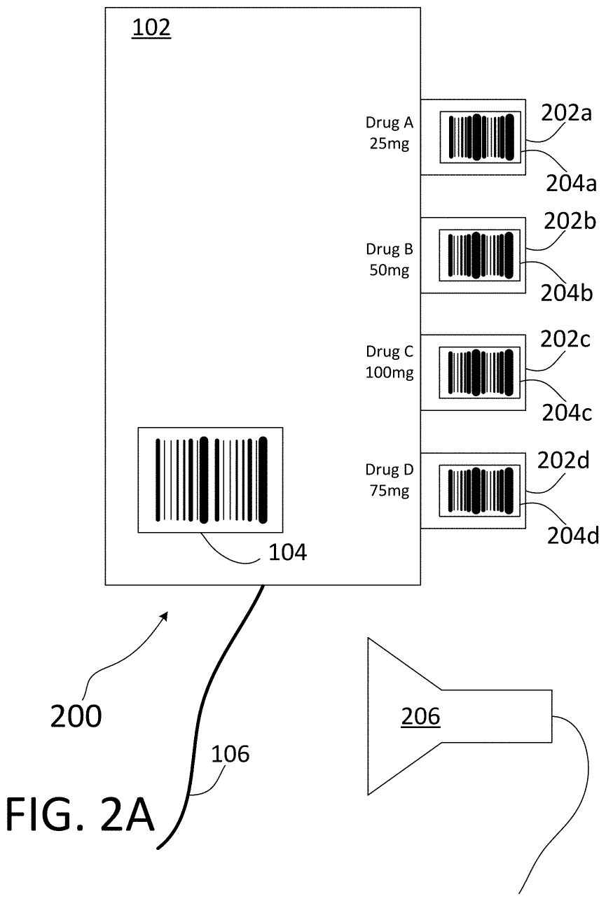

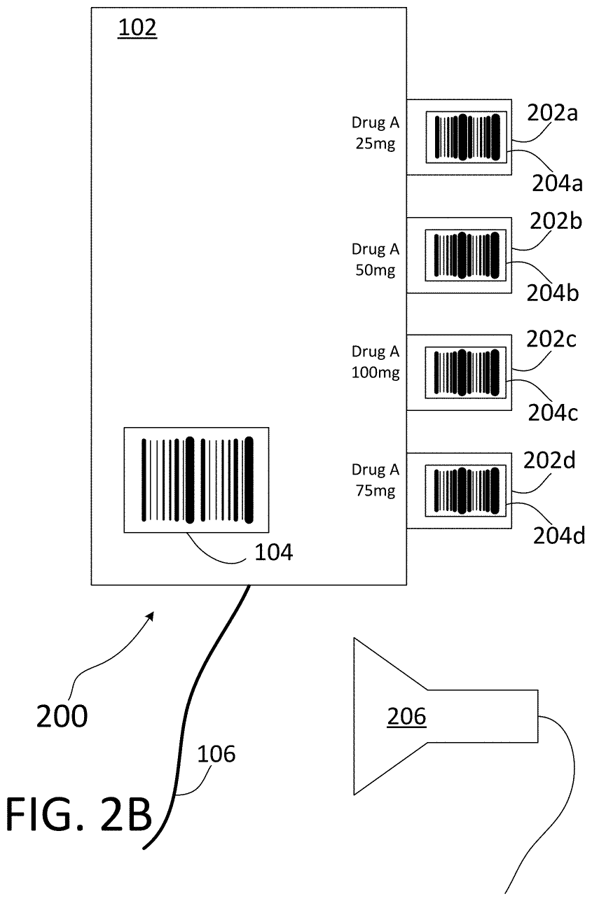

FIG. 1 illustrates a conventional medicament administration device, in this case, an intravenous solution bag (but may be any container such as a bottle, cartridge, etc.), into which, according to conventional processes, medicaments can be compounded (typically by a pharmacist measuring and/or intermixing multiple injectable products/medicaments) and inserted into bag 102 and then infused into a patient. As described above, there are numerous problems associated with the use of such a device/container. Shown in FIG. 1 is medication dispensing apparatus 100, which includes bag 102 (but which can be a bottle, syringe, cartridge, among other devices/containers), universal product code (UPC) label (or more conventionally referred to as a “barcode”) 104, and infusion line 106. The barcode 104 in the pharmacy/hospital environment is typically generated/provided by the pharmacy department and contains information pertaining to the contents of the bag 102, the patient's name, the patient's unit/room number, the patient's medical record number, product expiration date, infusion instructions such as rate of administration, etc. Aspects of the embodiments, as shown in FIGS. 2 and thereon, utilize one or more elements of the conventional apparatus 100, but with the differences as described herein that overcome the problems of the prior art.

Aspects of the embodiments can be used with a modularly assembled medical substance (modular format medicament assembly) because the modular assembly requires a plurality of containers to be provided to an end-user such as a nurse and/or a patient. The plurality of the containers may be commercially packaged/commercially prepackaged containers. Aspects of the embodiments include containers, software, processors, storage elements, memory elements, inputs, outputs, methods and/or devices that provide for user-friendly and efficient dispensing, record keeping, preparation, and verification of injectable medicaments housed in containers intended for the modular assembly format (modular format medicament assembly. The modular assembly format (modular format medicament assembly) is described/illustrated in U.S. patent applications Ser. Nos. 16/102,635; 16/102,651; 16/102,657; 16/102,666; 16/102,681; 16/102,698; 16/102,704; 16/102,713; 16/102,719 and may be referred to for understanding how the modular format medicament assembly system provides customized amounts of beneficial substance/medicaments to end-users, such as nurses, using commercially prepackaged/packaged containers without the need for manual manipulation (measuring/intermixing) of the beneficial substances/medicaments by pharmacists or other healthcare practitioners.

FIG. 2A illustrates modularly assembled medicament container/system (MAM container/system) 200 in which a plurality of containers 202 containing prescribed medicaments are attached/connected to intravenous (IV) bag 102 according to aspects of the embodiments.

In the embodiment of FIG. 2, MAM container 200 is an IV bag, and the attached containers are vials 202a-d; this is but one non-limiting example as other forms are possible to use in the different aspects of the embodiments. Each of containers 202a-d have a respective barcode 204a-d, although the use of other data containing elements is possible. Each of the respective barcodes 204a-d on respective ones of containers 202a-d can be scanned by scanning device 206 (alternatively referred to as a data acquisition device, as other means for storing and retrieving identical or substantially identical information can be used as well, according to aspects of the embodiments). Barcodes 204a-d can include such information as a unique identifier, a unique container/vial number (e.g. a serial number), a unique container/vial code, as well as information pertaining to the contents of the respective vial 202: thus, the use of barcodes 204 can be used in at least two ways. Pertinent information can be stored directly in the data element (drug type/identity/name, quantity/amount, date of manufacture, expiration date, name of manufacturer, among other useful information), as well as a record location indicator—the serial number (SN)—in which a data retrieval system attached to scanner 206 can use the SN to retrieve the same and/or different pertinent information regarding that particular vial 202. According to further aspects of the embodiments, barcode 204 and scanner 206 can be used in either or both manners. For the purposes of this description, barcodes 204 will be discussed as if they contain the pertinent information, although those of skill in the art can now appreciate that this is a non-limiting example only, and does not preclude other manners of using barcodes 204.

For example, barcode 204a of vial 202a attached to bag 102, when scanned by scanning device 206, can indicate that it stores information showing that 25 milligrams (mg) of a first drug (drug A) is contained in vial 202a. Barcode 204b of vial 202b attached to bag 102, when scanned by scanning device 206, can indicate that it stores information showing that 50 mg of a second drug (drug B) is contained in vial 202b. Barcode 204c of vial 202c attached to bag 102, when scanned by scanning device 206, can indicate that it stores information showing that 100 mg of a third drug (drug C) is contained in vial 202c. Barcode 204d of vial 202d attached to bag 102, when scanned by scanning device 206, can indicate that it stores information showing that 75 mg of a fourth drug (drug D) is contained in vial 202d.

Thus, in addition to whatever medicament/fluid/beneficial substance that might be contained in bag 102, an additional 250 mg (milligrams) of four different drugs (or the same drug) contained in vials 202a-202d are coupled/engaged to bag 102 and will be added/intermixed with the contents of bag 102.

According to aspects of the embodiments, MAM container 200 as shown in FIG. 2A includes four vials 202 attached to IV bag 102, which is then provided to a nurse prior to administration to a patient. In further aspects of the embodiments, IV bag 102 and the four vials 202 can be provided to an end-user such as a nurse or a patient as individual containers 202—i.e., not attached to IV bag 102, and then assembly/attachment of vials 202 would be required by the end-user/nurse (at the point of care). According to further aspects of the embodiments, and as discussed in greater detail below, vials 202 can be commercially packaged/prepackaged containers (supplied by a pharmaceutical/drug manufacturer/bulk volume distributor).

FIG. 2b illustrates modularly assembled medicament (MAM) container having 4 vials containing Drug A in different amounts attached to the MAM. Each of the four containers containing Drug A having a barcode containing at least one of an identity (name) of Drug A, an amount of Drug A in the respective container, and a unique identifier. The scanner (acquisition device) will scan the barcodes on each container and receive the information disposed/embedded/encoded in the barcode of the containers. The scanner may also optionally, or additionally, scan a barcode on the MAM container. The barcode on the MAM container may also contain (have embedded in/on it) information pertaining to the contents of the MAM container, the identity (name) of the contents of the MAM container, an amount of the contents of the MAM container, and/or a unique identifier. It is important to note the unique identifier on any of the containers mentioned in the herein specification may be numerical, alphabetical, alphanumeric, and combinations thereof. A drug manufacturer and/or pharmaceutical manufacturer may supply, in one or more embodiments, the unique identifier on all containers it manufactures, sells, and/or distributes that will differentiate each vial having the same drug in the same (or about the same) amount. In one or more embodiments, the unique identifiers mentioned anywhere in this specification may also form an industry (pharmaceutical industry) standard meaning that a plurality of drug companies, pharmaceutical companies, drug distributors, pharmaceutical distributors, pharmacies, hospital and/or retail pharmacies, compounding facilities, bulk manufacturers, bulk distributors, and combinations thereof may cooperate to form/provide an industry standard in which every/each drug having the same identity (generic and/or brand name and/or active ingredient) packaged in the same amount (or about the same amount) has a unique identifier which differentiates the vials/containers from other similar and/or identical labeled vials/containers irrespective of the drug company (or pharmaceutical manufacturer) that produces, manufactures, packages, labels, and/or distributes the drug/pharmaceutical product. In one non-limiting example, if drug company “Blue Pharmaceuticals” sells vials of Penicillin 5 million units and a second drug company “Green Pharmaceuticals” sells vials of Penicillin 5 million units then both companies “Blue Pharmaceuticals” and “Green Pharmaceuticals” may cooperate either directly or indirectly, for example through an industry intermediary (such as a working group, professional association, the Food and Drug Administration, a regulatory body, and the such) so as to label the Penicillin 5 million unit vials with different unique identifiers. Therefore, if a nurse/pharmacist has in stock (is using) Penicillin from different manufacturers to assemble a modular medicament assembly for a patient each of the vials from both Blue Pharmaceuticals and Green Pharmaceuticals will have a different unique identifier. The same unique identifier affixed to a Penicillin 5 million unit vial provided by Blue Pharmaceuticals cannot be affixed to a Penicillin 5 million unit vial provided/used by Green Pharmaceuticals. The unique identifiers used by Blue Pharmaceuticals and Green Pharmaceuticals must be different. Therefore, when the vials are scanned by the scanner (or any other acquisition device) the software that checks (cross-checks/compares) to prevent double (duplicate) scanning of the same vial/container will prevent double scanning (duplicate scanning) of the same vial regardless of the supplier/manufacturer of the Penicillin 5 million unit vial.

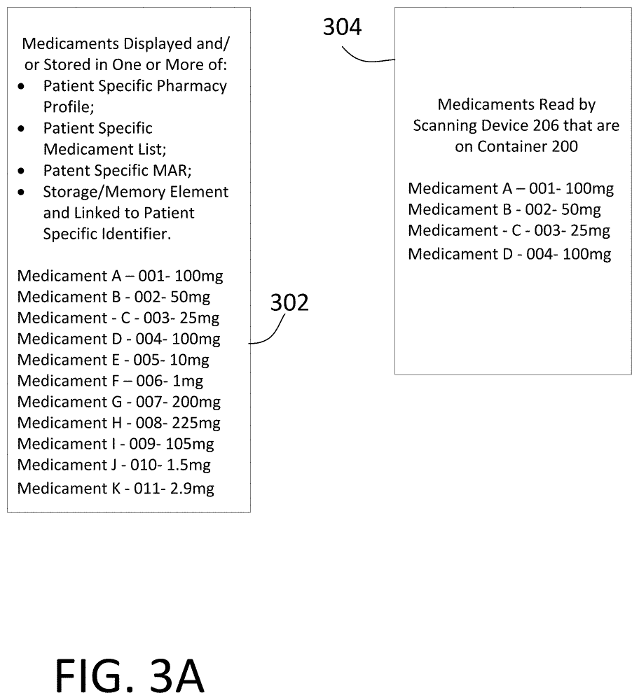

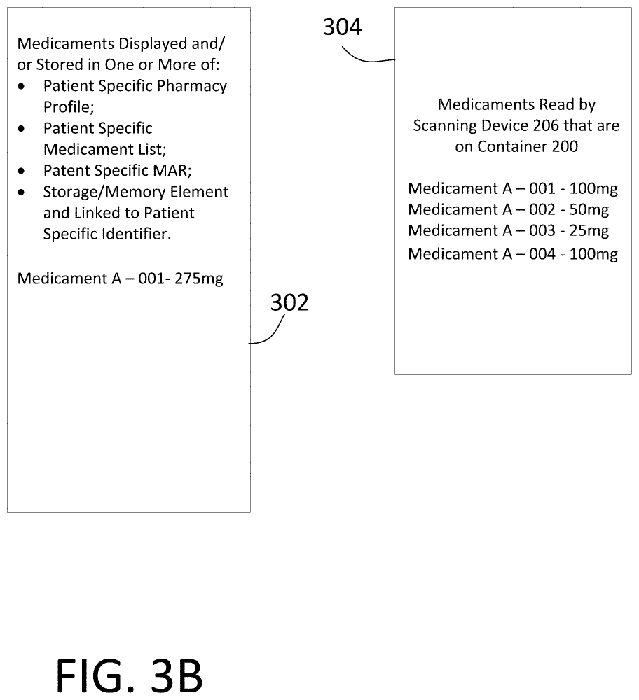

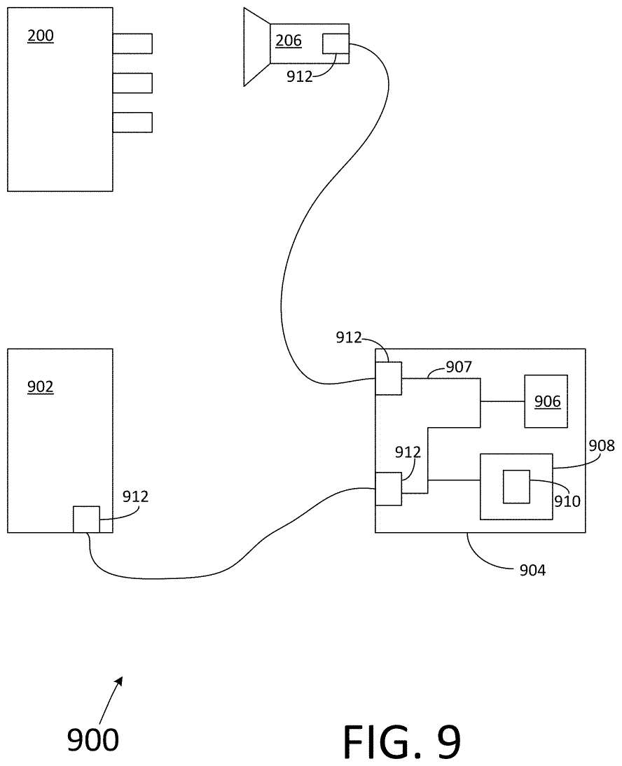

FIG. 3A illustrates modularly assembled medicament (MAM) container drug type and amount list 304 acquired by scanning device 206 in a list form and medication medicament prescription list (MMPL) 302 according to aspects of the embodiments. Once scanning device 206 (from FIG. 2) scans each barcode 204 (from FIG. 2) of respective two or more vials 202 (from FIG. 2), the information therein can be gathered together and presented as a list of data elements 304. Another version of the data elements is already present/stored in software associated with an associated system—the MAR—as a final dosage amount in regard to the particular patient. According to aspects of the embodiments, a system as described herein (medicament administration verification system (MAV system) 900; discussed and described in regard to FIG. 9, below) can include one or more processors that receive data from scanning device 206 (e.g., data stored in barcode 204). The one or more processors can then compare stored data elements 304 from barcodes 204 on vials 202 against MMPL 302 from the MAR and verify that the types and quantities of drugs being administered by a nurse or other caregiver match the prescribed information that is stored in the MAR.

According to aspects of the embodiments, stored data elements 304 can be linked to a patient specific profile, wherein the patient specific profile can be a patient specific pharmacy profile, a patient specific medication list, a patient specific nursing profile, a patient specific medication administration record, a patient specific medication profile list, among others. According to further aspects of the embodiments, stored data element 304 can be linked to a patient name, a patient identification number, a medical record number, a patient's date of birth, and/or to any other type of patient specific identifying information.

FIG. 3B illustrates element 302 where medicament A is entered by a prescriber as one value (final dosage amount) of 275 mg into a prescription (not shown), a patient specific prescription (not shown), patient profile, a patient specific profile, a patient specific pharmacy profile, a medicament list, a patient medicament list, a medication administration record MAR, a medicament prescription list MMPL, a storage/memory element linked to a patient specific identifier, and combinations thereof. Data element 302 may be stored on a computer, on a server, in the cloud, on a storage element of a computer, on a memory element of a computer, on a handheld device, and combinations thereof. Data elements 304 were acquired by the acquisition device 206 (scanner) in FIG. 2B. Data elements 304 are all the same medicament A in four different amounts 100 mg, 50 mg, 25 mg, and 100 mg. Data element 302 is then compared to data elements 304 to determine if the same or different final dose is provided 304 compared to the final dose that was prescribed in 302. Adding (summing) the four doses contained in containers 202a-202d of FIG. 2B as shown in 304 (100 mg+50 mg+25 mg+100 mg=275 mg total dose) which is the same/equivalent to the 275 mg prescribed dose shown in 302. This is a match condition since 302 contains a 275 mg prescribed dose that matches 304 provided dose of 275 mg.

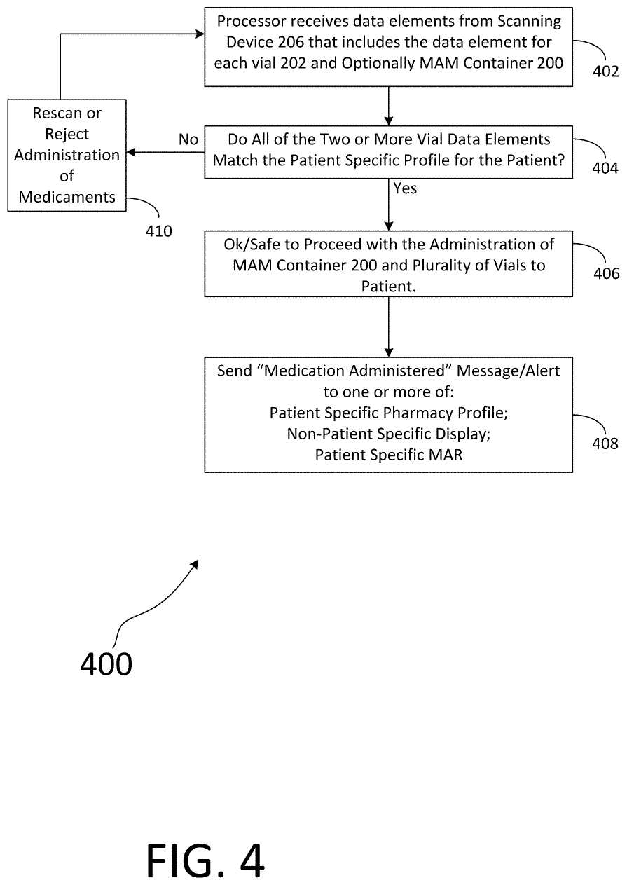

Attention is now directed towards FIG. 4. FIG. 4 illustrates a flow chart of method 400 for comparing medication administration record (MAR) medicament prescription list (MMPL) 302 versus modularly assembled medicament (MAM) container drug type and amount list (stored data elements) 304 according to aspects of the embodiments. According to aspects of the embodiments, stored data elements 304 can be compared to MMPL 302. That is, stored data elements list 304 is matched to MMPL 302 using the one or more processors and/or software as shown and described in regard to FIGS. 9-11, among others.

Method 400 begins with method step 402. In step 402, the software, processing device, processing and storage device, computer, smartphone, tablet, server, application (app), cloud based software/app, and/or a processor receives data elements 304 from scanner 206 that includes at least one of drug type and amount stored in barcode 204s for each of vials 202s, and optionally data encoded and stored in barcode 104 of container 102; the latter generally includes at least patient related data such as name, and other identifying information but may also include at least one of an identity of the contents of container 102 and an amount(s) of the contents of container 102. Step 402 may also in some embodiments include receiving a data element from MAM container 200. Step 402 has a computer, a server, a processing device, and/or a processor receiving data elements from the scanning device 206 but it is explicitly stated here that in FIG. 4 as well as anywhere in the herein specification a processor may be any other device and/or component of a computer and/or software that processes data elements and/or other information. The systems, devices, and methods disclosed anywhere in FIG. 4 and anywhere in the herein specification are not limited to a processor (the term processor is used arbitrarily and any device and/or component of a computer/server/handheld device that performs similar functions as a processor may replace the processor). In decision step 404, MMPL 302 is retrieved and compared to stored data elements 304. If not all of the data matches, meaning one or more of a drug type and/or quantity is different, or a drug is missing, or redundant (e.g., one or more vials 202 were missed or one or more vials 202 were multiplicatively scanned), then method 400 proceeds to method step 410 (“No” path from decision step 404), and alerts the user (e.g., nurse or other caretaker) to re-scan all of the barcodes 204 of all of the vials 202, and optionally re-scan barcode 104 of bag 102, and checks once again in decision step 404 if the information scanned matches that as found in the MAR (e.g., MMPL 302). According to aspects of the embodiments, the repetition of steps 402, 404 and 410 can continue indefinitely, or, can be limited to a certain number of attempts. In one or more embodiments, in step 404 data elements 302 are retrieved from that are displayed and/or stored on one or more of a patient specific pharmacy profile, a patient specific profile, a patient specific medicament list, a patient specific MAR, a storage element, a memory element, a storage element linked to a patient specific identifier, a memory element linked to a patient specific identifier, and combinations thereof. The data elements 302 are then compared to stored data element 304 obtained from the acquisition (scanning) device. If not all of the data matches, meaning one or more of a drug type and/or quantity is different, or a drug is missing, or redundant (e.g., one or more vials 202 were missed or one or more vials 202 were multiplicatively scanned), then method 400 proceeds to method step 410 (“No” path from decision step 404), and alerts the user (e.g., nurse or other caretaker) to re-scan all of the barcodes 204 of all of the vials 202, and optionally re-scan barcode 104 of bag 102, and checks once again in decision step 404 if the information scanned matches that as found in the MAR (e.g., MMPL 302). According to aspects of the embodiments, the repetition of steps 402, 404 and 410 can continue indefinitely, or, can be limited to a certain number of attempts.

If, however, all of the data matches between that of MMPL 302 and stored data elements 304, then method 400 proceeds to method step 406 (“Yes” path from decision step 404). In method step 406, it is determined that it is safe/OK to proceed with administration of the modularly assembled medicament. In one or more embodiments, method step 406, it is determined that it is safe/OK to proceed with administration of the modularly assembled medicament container and a plurality of vials 202. Then, in method step 408, an alert/output is sent to the caretaker alerting the caretake that it is safe to proceed with the administration of the contents of MAM container 200 and all attached vials 202 to the patient.

Each of the alerts/outputs generated by steps 410 and 408 can be audible, visual, tactic, or any combination thereof. In addition, such alert, whether a verification or alert not to proceed can be added to, and shown on the profile of a patient, can be stored or displayed on a patient specific mediation list, and/or can be stored in/on the patient specific mediation administration record MAR of the patient, or any combinations thereof. According to aspects of the embodiments, the verification alert or the alert not to proceed may be stored on a storage element, on a memory element, in/on a computer, in/on a handheld device (such as a smartphone), in/on an application (app), on a server, in the cloud, and combinations thereof.

According to aspects of the embodiments, the output that can be generated that alerts a user that is it safe (OK) to proceed with administration of the modular medicament assembly can be in the form of a visual element such as a check mark on a patient medication record, a check mark on a patient profile, a check mark on a patient specific medication list, and/or a check mark on a patient medication administration record and/or on any other patient specific screen. The check mark can be green in color (i.e., the check mark can be color coded). According to still further aspects of the embodiments, the visual element can be an “X,” indicating that it is not safe to dispense the medicaments; such an “X” can be red in color (or virtually any other color). The “X” can be displayed on one or more of a patient medication record, a patient profile, a patient specific medication list, a patient administration record, a patient specific medication administration record, and any other patient specific screen, according to aspects of the embodiments.

As discussed above, bar codes 204 can include data elements stored therein. The data elements can be a unique identifier that is different for each vial 202, and which can take the form of a code, a numeric code, an alphabetical code, an alphanumeric code, a barcode and combinations thereof. According to aspects of the embodiments, medicament/drug in each of the attached vials 202 can be the same medicament, or different medicaments. According to aspects of the embodiments, medicament/drug in each of the attached vials 202 may take the form of a nutritional substance, a diagnostic substance, and/or any other beneficial substance.

According to further aspects of the embodiments, as described above, the stored data elements in bar codes 204 can be entered/scanned into a computer and/or software program with scanner 206; however, according to further aspects of the embodiments, entry of the data element and/or unique identifiers can be performed manually by typing in a code and/or a unique identification code by a user using a keyboard, a keypad, and/or a touch screen keypad/keyboard.

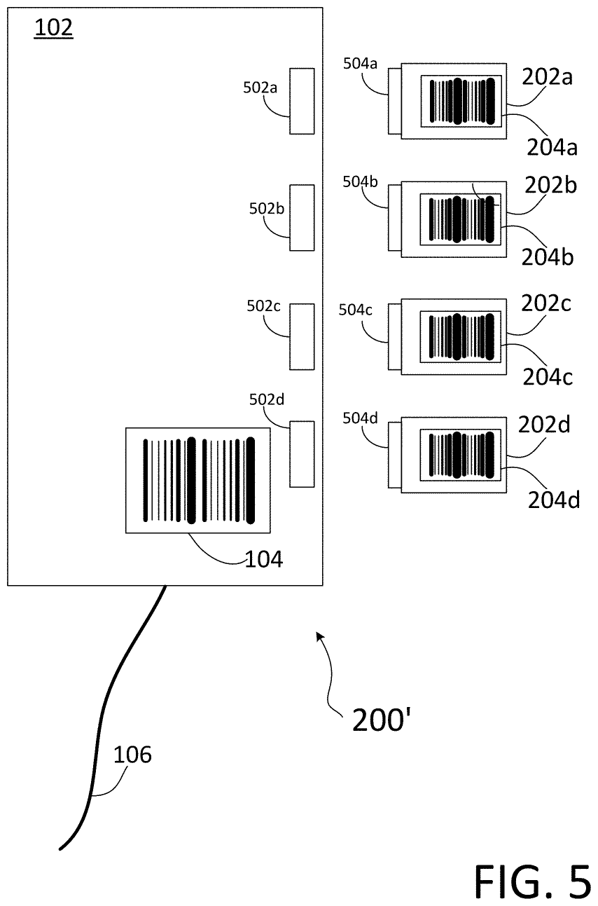

FIG. 5 illustrates un-assembled modularly assembled medicament container 200′ in which a plurality of containers 202 containing prescribed medicaments can be attached/connected to IV bag 102 according to aspects of the embodiments. The end-user can assemble vials 202 at the point-of-care. Scanner 206 (not shown) can scan the respective barcodes 204 of each of the plurality of vials 202 such that the medicament information can be forwarded to a system, software, processing device, and/or processor for processing, as described above in regard to FIG. 4. The system, software, processing device, and/or processor is also further adapted to receive patient specific data as stored or referenced in barcode 104; if the latter, i.e., barcode 104 merely contains referring information, e.g., not the prescription, then the system, software, processing device, processor, and/or methods according to aspects of the embodiments can then use the identifier stored in barcode 104 to retrieve patient specific information, such as a patient specific profile, a patient specific medication list, a patient specific medication administration record (MAR), a prescription, a patient specific prescription, a patient specific storage element, and/or a patient specific memory element, among other types of information. Cross-checking and verification then occurs, as described above.

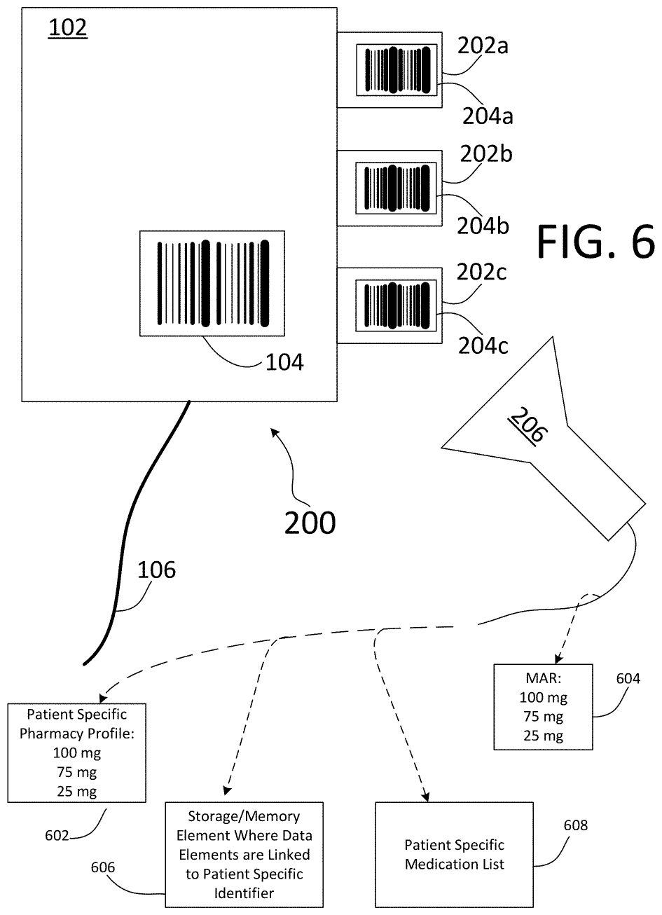

FIG. 6 illustrates a further embodiment of MAM container 200 with three vials 202a-c attached thereto, and two destinations of data that can be obtained by scanner 206 according to aspects of the embodiments. As those of skill in the art can now appreciate, the examples shown in FIG. 6 are non-limiting examples only. Vial 202a contains 100 mg of medicament A, vial 202b contains 75 mg of medicament B, and vial 202c contains 25 mg of medicament C. Scanner 206 scans barcodes 204a-c on vials 202a-c, respectively, and optionally barcode 104 on bag 102, and the data can be sent to one or more of several different locations. According to a first aspects of the embodiments, the “amount” data (i.e., the data that was scanned that reflects the amount in the scanned vial) as scanned can be sent to a patient specific pharmacy profile 602 where a list of the container amounts (100 mg, 75 mg, 25 mg) can be stored/displayed. According to further aspects of the embodiments, the same “amount” data can also, or in the alternative, be sent to a patient specific MAR 604, where a list of the container amounts (100 mg, 75 mg, 25 mg) can be stored/displayed. According to further aspects of the embodiments, the same “amount” data can also, or in the alternative, be sent to storage/memory element 606, and patient specific medication list 608. According to further aspects of the embodiments, in any or all of these records (602,604, 606, 608), a total amount of medicaments contained in vials 202a-c can be calculated, and also stored/displayed in any or all of records 602, 604, 606, and 608 (e.g., the total amount listed or stored/displayed would be 200 mg since 100 mg from vial 202a+75 mg from vial 202b+25 mg from vial 202c=200 mg). According to still further aspects of the embodiments, a list of the container amounts as scanned by scanner 206 can be stored/displayed under any or all of records 602, 604, 606, 608. Additionally, or optionally, a list of container amounts and/or a summed/total amount obtained from barcodes 204a-204c by acquisition device/scanner 206 can be stored in a storage element and/or a memory element of a computer, a server, a handheld device (such as a smartphone), software, an application (app), in the cloud, and combinations thereof. The list of container amounts and/or summed/total amount that has been stored in the storage element and/or memory element may be retrieved and displayed on/under a patient specific display such as (but not limited to) a patient specific profile, a patient specific medication administration record, a patient specific medication list, and combinations thereof. According to aspects of the embodiments, the list of container amounts and/or summed/total amount is linked (electronically linked) to a patient specific identifier and stored in/on a storage element and/or memory element along with the patient specific identifier. The patient specific identifier may be (but is not limited to) the patient's name, date of birth, weight, height, room number, unit number, medical record number, identification code, social security number, and combinations thereof. The patient specific pharmacy profile 602 may in some embodiments be any patient specific profile, a patient specific medication list 608, a patient profile, a nursing profile, a patient specific nursing profile, and combinations thereof.

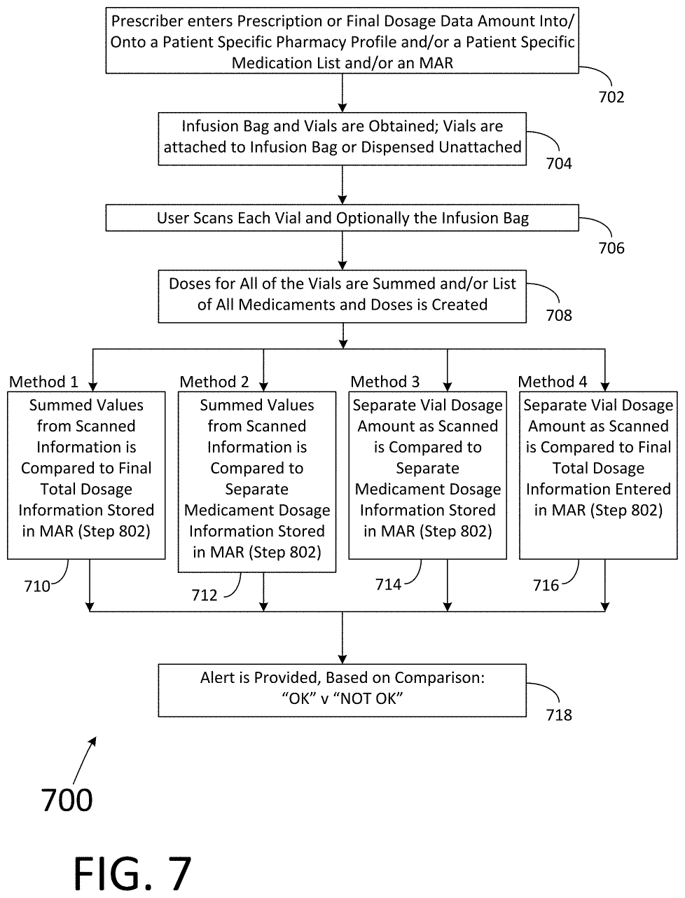

FIG. 7 illustrates a flow chart of method 700 for comparing data elements obtained from two or more containers comprising part of the modularly assembled medicament container (modular format medicament assembly) as shown in FIGS. 2A and 2B against a medication administration record prescription list according to aspects of the embodiments. According to aspects of the embodiments, the data elements obtained from the two or more containers comprising part of the modularly assembled medicament container (modular format medicament assembly) as shown in FIGS. 2A and 2B may also be compared against at least one of a patient specific profile, a patient specific medication list, a prescription, a patient specific prescription, and patient specific stored information (not shown). FIG. 7 may be, in one or more embodiments, a computer implemented method. The computer implemented method may comprise/require the use of a processor, a server, a memory/storage device, and other devices/peripherals typically contained and/or used by a computer when implementing the method. Optionally, or additionally, in one or more embodiments FIG. 7 may comprise a software implemented method. FIG. 7 may also, in one or more embodiments, comprise a computer implemented method and a software implemented method. According to aspects of the embodiments, the patient specific medication list and/or the medication administration record MAR may be a patient specific medication administration record, a patient specific prescription list, and/or any other patient specific display and/or storage/memory element that contains a patient specific list of medications the patient is prescribed/receiving. According to aspects of the embodiments, FIG. 7 illustrates in greater detail several steps of method 400 as shown and described in regard to FIG. 4. Since several of the steps of method 700 have already been discussed before, either in the context of FIG. 4 and method 400 or elsewhere, then a detailed discussion of the same shall be omitted in fulfillment of the dual purposes of clarity and brevity.

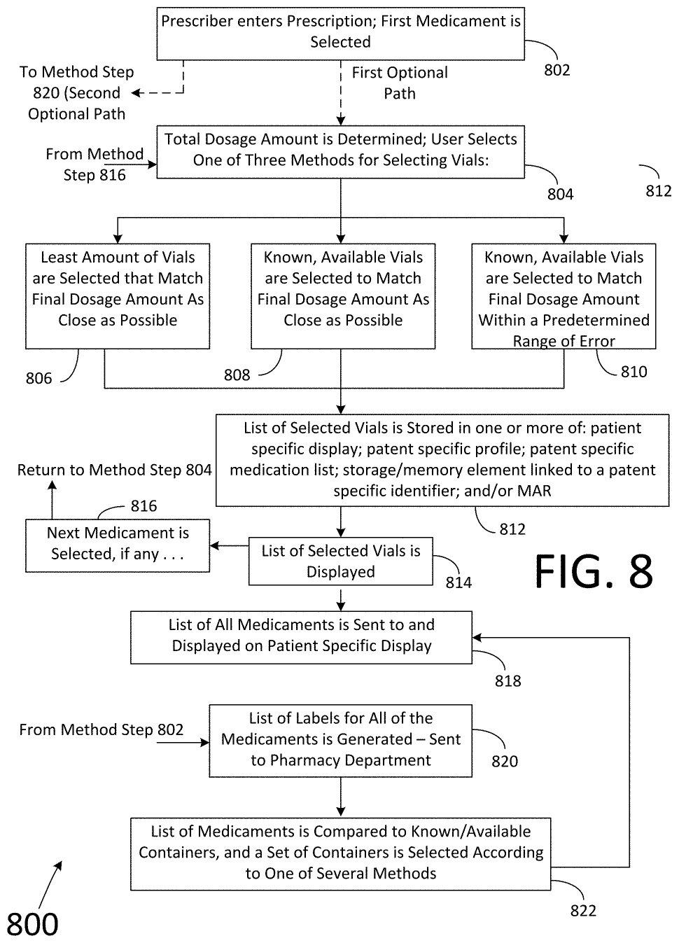

Method 700 begins with step 702 in which a prescriber enters prescription and/or final dosage data amount into/onto a patient specific pharmacy profile and/or a patient specific medication list and/or into the MAR of a patient and/or a prescription for a patient, and/or a prescription entry screen/display for a patient. It is explicitly stated here, that according to aspects of the embodiments, the prescriber is not limited to entering the prescription information into the MAR of a patient but may alternately, or optionally, or additionally, enter prescription information into a patient profile, a patient specific profile, a patient medication list, a patient specific medication list, and combinations thereof. In step 702 the prescriber may enter the prescription in a final dosage amount form (for example, Penicillin 10 million units administered intravenously every 8 hours) or in a list form (for example, Penicillin one 5 million unit vial+Penicillin one 5 million unit vial both vials administered intravenously every 8 hours). MAV system 900 updates and/or stores the new prescription information as either or both a list of medicaments and respective dosages, as well as a total dosage amount for all of the medicaments. In step 704 a nurse/pharmacist/caretaker obtains IV bag 102 and attaches to it the prescribed medicaments 202 with the prescribed dosages to IV bag 102, optionally nurse/pharmacist/caretaker obtains IV bag 102 and supplies prescribed medicaments 202 with the prescribed dosages along with bag 102 to an end user. Vials 202 may be dispensed unattached to IV bag 102, wherein vials 202 will be assembled/attached to IV bag 102 at the point of care by the patient and/or caregiver, according to aspects of the embodiments. According to aspects of the embodiments, the system and method described herein can ascertain which medicament containers to obtain, and this method is discussed in greater detail in regard to FIG. 8.

In method step 706 the user/caretaker uses scanner 206 to scan each barcode 204 of each attached vial 202, and optionally scans barcode 104, and in method step 708, MAV system 900 creates a list of scanned medicaments and dosages and/or a total sum of the medicament dosages. In other words, in step 708, MAV system 900 creates at least one of a list of scanned medicaments/dosages and a total sum (summed) of scanned medicaments dosages.

In decision step 404, of method 400, it was determined whether all of the data matches—i.e., a comparison was made between the scanned data from vials 202 and the prescription as maintained by the MAR (and/or patient profile, and/or patient specific profile, and/or patient specific medication list, and/or prescription, and/or patient specific prescription). The step of matching determined whether one or more of a drug type or quantity was different, or a drug was missing, or redundant (e.g., one or more vials 202 were missed or one or more vials 202 were multiplicatively scanned). According to aspects of the embodiments, there are at least four methods of matching and/or comparing data elements 304 (which were obtained by the scanner 206 from the modular medicament assembly/MAM) with that of the medication administration record MAR and/or patient specific pharmacy profile and/or patient specific MAR and/or patient profile and/or patient specific medication list and/or patient specific storage/memory element and/or storage/memory element linked to a patient specific identifier as shown in 302 of FIG. 3B; these are shown a steps 710, 712, 714, and 716. Each of these will now be discussed in detail, and are referred to as method 1, method 2, method 3, and method 4.

According to aspects of the embodiments, the processing device/processor/software/computer/method of MAV system 900 can perform a mathematical calculation that may be (but is not limited to) addition and/or multiplication on at least one of data elements 302 of FIG. 3B and data elements 304 of FIG. 3B. In regard to the four methods of steps 710, 712, 714, and 716, a list is generated of the medicaments and their dosage quantities/amounts of stored data elements 304 and MMPL 302, and optionally, or alternately, a final total dosage amount.

In optional method step 710, the summed values of the scanned information, data elements 304, is compared to the total dosage amount of MMPL 302. That is, if four medicaments were provided and their vials scanned, as described in regard to FIGS. 2A and 2B, the four vial amounts are added (SUMMED) and their total dosage amount of 250 mgs is compared to the total final dosage of MMPL 302. If the totals match then the alert is provided, in step 718, that it is safe for the user/caretaker to proceed to administer the medicaments, as described above in regard to method 400 and step 406. If, however, the totals do not match, then an alert is provided, again in step 718, that it is not safe to proceed to administer the medicaments, as described above in regard to method 400 and step 410, and either a rescan is performed or no medicaments are administered until the prescription and/or medicaments can be verified.

In optional method step 712, the summed values of the scanned information, stored data elements 304, is compared to the respective individual amounts of the medicaments of MMPL 302 (in this non-limiting example, there are four medicaments). That is, if four medicaments were provided and their vials scanned, as described in regard to FIG. 2, the four scanned medicament amounts are added/calculated (SUMMED) and their total dosage amount of 250 mgs is compared to each separate dosage of each separate medicament of MMPL 302. Certain mathematical operations are performed to verify the medicaments and dosages. For example, for each medicament and dosage, the medicament can be matched, (e.g., medicaments A are both present in each list), and if okay, then the individual dosage from MMPL 302 is subtracted from the total of the summed values of stored data elements 304. Other mathematical operations can also be used to achieve substantially identical results, according to aspects of the embodiments. If a match occurs then the alert is provided, in step 718, that it is safe for the user/caretaker to proceed to administer the medicaments, as described above in regard to method 400 and step 406. If, however, the totals do not match, then an alert is provided, again in step 718, that it is not safe to proceed to administer the medicaments, as described above in regard to method 400 and step 410, and either a rescan is performed or no medicaments are administered until the prescription and/or medicaments can be verified.

In optional method step 714, the individual amounts/values and/or types of medicaments of the scanned information from scanner 206, data elements 304, is compared to the respective individual amounts of the medicaments of MMPL 302 (in this non-limiting example, there are four medicaments). That is, if four medicaments were provided and their vials scanned, as described in regard to FIGS. 2A and 2B, their individual dosage amounts of 25 mg of drug A, 50 mg of drug B, 100 mg of drug C, and 75 mg of drug D is compared to each separate dosage of each separate medicament of MMPL 302. In this case, a simple comparison (cross-check) can be performed to verify the medicaments and/or dosages. Other mathematical operations means of matching can also be used to achieve substantially identical results, according to aspects of the embodiments. If a match occurs then the alert is provided, in step 718, that it is safe for the user/caretaker to proceed to administer the medicaments, as described above in regard to method 400 and step 406. If, however, if any of the medicaments of 302 and 304 do not match, then an alert is provided, again in step 718, that it is not safe to proceed to administer the medicaments, as described above in regard to method 400 and step 410, and either a rescan is performed or no medicaments are administered until the prescription and/or medicaments can be verified.

In optional method step 716, the individual values and/or types of medicaments of the scanned information from scanner 206, data elements 304, is compared to the total summed dosage amount of MMPL 302 (in this non-limiting example, there are four medicaments). That is, if four medicaments were provided and their vials scanned, as described in regard to FIGS. 2A and 2B, their individual dosage amounts of 25 mg of drug A, 50 mg of drug B, 100 mg of drug C, and 75 mg of drug D is compared to the total dosage of medicaments of MMPL 302 of 250 mg, and certain mathematical operations are performed to verify the medicaments and dosages. For example, for each medicament and dosage, the medicament identity/name can be matched (e.g., medicaments A are both present in each list), and if okay, then the individual dosage from stored data elements 304 are subtracted from the total of the summed values of MMPL 302. Other mathematical operations can also be used to achieve substantially identical results, according to aspects of the embodiments. If a match occurs then the alert is provided, in step 718, that it is safe for the user/caretaker to proceed to administer the medicaments, as described above in regard to method 400 and step 406. If, however, the totals do not match, then an alert is provided, again in step 718, that it is not safe to proceed to administer the medicaments, as described above in regard to method 400 and step 410, and either a rescan is performed or no medicaments are administered until the prescription and/or medicaments can be verified.

FIG. 8 illustrates a flow chart of method 800 for selecting one or more medicament vials 202 for use with MAM container 200 shown in FIGS. 2A and 2B according to aspects of the embodiments. According to aspects of the embodiments, method 800 may be a computer implemented method. According to aspects of the embodiments, method 800 may be a software implemented method. According to aspects of the embodiments, method 800 may be a computer and a software implemented method. Method 800 begins with step 802 in which a prescriber enters a prescription. The prescription can be referred to as an order, an electronic order, or an electronic prescription, among other describers. The prescriber enters the prescription as a final dosage amount of 175 mg of a first medicament. According to aspects of the embodiments, there can be multiple medicaments, or just a single medicament. By way of non-limiting example, the dosage amount in this case is 175 mg. There are two optional paths method 800 can proceed via: the first optional path can be referred to as the automated path, and the second optional path can be referred to as the manual path. Each shall be described in turn, beginning with the first optional, or automated path.

Method 800 then proceeds to step 804 from step 802. In method step 804, method 800 may optionally store the prescription/prescription total dosage amount/prescription list of medicament amounts, and then one of three methods are selected to determine how vials 202 are to be selected.

In a first option, method step 806, method 800 selects the least amount of vials 202 that matches the final dosage amount/quantity either exactly and/or as close as possible. That is, in the example, the prescription is for 175 mg of medicament A. System 900 and method 800 know that there is, for example, a single vial containing 175 mg of medicament A, and eight vials of medicament A containing 25 mg each. In method step 806, system 900 and method 800 select the least amount of vials that match 175 mg, which is the first vial 202 containing the 175 mg. If, by way of non-limiting example, the 175 mg vial did not exist, but there were an additional two 100 mg vials of medicament A, one 100 mg vial 202 and three 25 mg vials 202 would be selected. Method step 806 selected the least amount of vials that can be selected that match as close as possible the prescribed final dosage for the medicament. If it cannot match the final dosage as close as possible, then method step 806 indicates a failure and stops. Once the final prescribed dosage is obtained as close as possible, however, method 800 proceeds to method step 812, discussed below.

In a second option, method step 808, method 800 selects a number of vials 202 that matches the final dosage quantity as closely as possible. That is, in the example, the prescription is for 175 mg of medicament A. System 900 and method 800 know that there is, for example, a single vial containing 175 mg of medicament A, and eight vials of medicament A containing 25 mg each. In method step 808, system 900 and method 800 select a number of vials that match 175 mg as close as possible, which is the first vial 202 containing the 175 mg. If, by way of non-limiting example, the 175 mg vial did not exist, but there were an additional two 100 mg vials of medicament A, one 100 mg vial 202 and three 25 mg vials 202 would be selected. Method step 808 selected the least amount of vials that can be selected that match the prescribed dosage for the medicament as closely as possible—in this case, an exact match. If it cannot match the dosage as close as possible with the least number of vials, then method step 808 will match the prescribed dosage as closely as possible regardless of the number of vials. In a further example, there are the following vials 202 of the prescribed medicament: two 100 mg vials, three 50 mg vial, and three 20 mg vial. A first group that is close to the prescribed amount, 175 mg, is the two 100 mg vials, which equals 200 mg, a difference of 25 mg. But a second group can be selected, consisting of one 100 mg vial, one 50 mg vial and one 20 mg vial, which produces 170 mg. As those of skill in the art can appreciate, an overdosage of a prescribed medicament can be problematic, or it can be innocuous, depending on the circumstances of the patient and prescribed medicament. The aspects of the embodiments in regard to method 800 is concerned with matching a dosage, in one of several different means, and not for the propriety of administering a dosage that is not as prescribed; such decisions are the province of the user/caretaker/physician. Thus, in this example, system 900 and method 800 and step 808 would select the group consisting of the 100 mg, 50 mg and 20 mg vials, as such dosage amounts to 170 mg, which amounts to a difference of 5 mg, versus 25 mg with the two 100 mg vials. Once the prescribed dosage is matched as close as possible in regard to the vials known to system 900 and method 800, method 800 proceeds to method step 812, discussed below.

In a third option, method step 810, method 800 selects the least amount of vials 202 that matches the final dosage quantity within a known, predetermined, range of error. That is, in the example, the prescription is for 175 mg of medicament A. Such range of error can be 10%, 15%, 20%, or whatever range of error is determined to be appropriate for the medicament; as such, the range of errors can be different for different medicaments, or it can be a universally applied range of errors. According to still a further aspect of the embodiments, system 900 and method 800 can select the set of vials that are within a range of error, and also minimizes that error. According to aspects of the embodiments, the predetermined range of error/variance from the prescribed dose may be set at 10% as a default error/variance since 10% variance is typically acceptable in the healthcare/pharmacy setting. Optionally, or additionally, the predetermined range of error/variance may be adjusted/preset by a superuser of the software/computer, a healthcare institution, or a healthcare provider in a management/decision-making role. By way of example, if in a first group of medicaments, there are four vials, one 150 mg, and three 10 mg vials, and the prescribed dosage of the medicament is 175, and the range of error is 5%, then this group can be selected (175-170=5 mg, 5/175=2.8% error, which is within 5% error). If, however, the group also included an additional 3 mg vial, then the selected group would be the 150 mg vial, two 10 mg vials, and the 3 mg vial, and the total would be 173 mg, and that error would be 1.14%, which is again within the 5% predetermined error range. The software may be configured/programmed to select the 173 mg dose over the instead of the 170 mg dose since the 173 mg dose has a smaller percentage error/variance from prescribed dose. However, the software may also in some embodiments be configured/programmed to select either the 170 mg or the 173 mg dose without any preference of one dose over the other dose since both doses are within the 5% predetermined range/variance. The predetermined range/variance may be±meaning a ±5% predetermined range/variance may mean a final dose is supplied that is either 5% above or 5% below the prescribed final dose.

As those of skill in the art can appreciate, an overdosage of a prescribed medicament can be problematic, or it can be innocuous, depending on the circumstances of the patient and prescribed medicament. The aspects of the embodiments in regard to method 800 are concerned with matching a dosage, by one of several different means, and not for the propriety of administering a dosage that is not as prescribed; such decisions are the province of the user/caretaker/physician. If system 900 and method 800 cannot match the dosage within the range of error, then method step 810 indicates a failure and stops. Once the prescribed dosage is matched as close as possible in regard to the vials known to system 900 and method 800 within the predetermined, known range of error, method 800 proceeds to method step 812, discussed below.

According to aspects of the embodiments, the systems and methods described herein can be adapted to optimize the selection of vials to provide a final dosage amount using the smallest/lowest number of vials 202 and to provide a final dosage amount as close as possible to the prescribed final dosage amount (or within a predetermined range). In one non-limiting example, a dose of drug A in the amount of 255 mg is prescribed and there are the following vials/containers of drug A that are commercially available or are in pharmacy stock available to be dispensed: three 100 mg, ten 50 mg, five 25 mg, five 5 mg, and ten 1 mg, the processing device/computer/handheld device/software/processor may be programmed to select two 100 mg vials+one 50 mg vial+one 5 mg vial=final dose of 255 mg which is the least number of vials/containers that will provide a final dose as close as possible to the prescribed dose of 255 mg. According to aspects of the embodiments, combinations of steps 806, 808, and 810 are also contemplated. According to further aspects of the embodiments, system 900 and the methods described herein can be adapted to scan and/or choose vials 202 from a list of commercially available vials 202. According to further aspects of the embodiments, system 900 and the methods described herein can be adapted to scan and/or choose vials 202 from a list of vials 202 that are in stock in the pharmacy, in a pyxis machine, in an automated dispensing machine, or other healthcare setting in which vials 202 will be used. According to aspects of the embodiments, the list of vials 202 that are in stock needs to be periodically and/or continuously updated by the staff to ensure that an adequate selection of vials 202 in varying dosage amounts is in stock for optimal selection and delivery of the appropriate vials.

Following each of steps 806, 808, and 810, method 800 proceeds to method step 812. Once one or more vials 202 has been selected, the selected vials 202 are added to a list and/or a list is created of the selected vials 202 and/or selected vials 202 are stored in/on one or more of a patient specific display, a patient specific profile, a patient specific medication list, a storage/memory element (not shown), a storage/memory element linked to a patient specific identifier, a medication administration record, a patient specific medication administration record, a prescription (not shown), a patient specific prescription (not shown), in method step 812. It is explicitly stated that the one or more vials 202 instead or in addition to being added and/or stored on the MAR in step 812 may also, or optionally/alternately/or additionally, be added and/or stored on a patient profile, on a patient specific profile, on a patient specific medication list, on a patient specific display, on/in a storage element, on/in a memory element, on/in a patient specific storage/memory element, on/in a storage/memory element linked to a patient specific identifier, on a MAR, and combinations thereof. The list of selected vials 202 can be displayed as shown in step 814, and then the next medicament (step 816), if any, is selected, and method 800 repeats steps 804-814 until all of the medicaments and vials 202 have been selected, and displayed on a list, in step 814. List of selected vials displayed on step 814 may be displayed on a patient profile, on a patient specific profile, on a patient medication list, on a patient specific medication list, on an order entry screen, on a patient specific order entry screen, on a patient specific display, on a non-patient specific display, on a prescription, on a patient specific prescription, and combinations thereof. Following the final occurrence of method step 814, method 800 proceeds to method step 818, in which a list of all of the medicaments and respective vials 202 is stored and/or displayed in/on a patient profile, a patient specific profile, a patient medication list, a patient specific medication list, a patient specific display, a non-patient specific display, a storage element, a memory element, the MAR of the patient, a prescription, a patient specific prescription, and combinations thereof.

According to further aspects of the embodiments, system 900 and method 800 can produce an output element (list), which can then be displayed and/or stored on a patient specific display such as a patient profile, a patient specific profile, a medication administration record MAR, a patient specific medication administration record MAR, a patient's medication list, and/or any other patient specific display, storage, a prescription, and/or memory element.

Returning back to method step 802, the second optional path will now be discussed; this is the “manual path.” In step 802 a prescriber enters a prescription. The prescription can be referred to as an order, an electronic order, a digital prescription, and/or an electronic prescription, among other describers. The prescriber enters the prescription as a final dosage amount of 175 mg of a first medicament. According to aspects of the embodiments, there can be multiple medicaments, or just a single medicament. In method step 820 any or any combination of a computer, an application (app), pharmacy software, optionally a processor, generates at least one and/or a list of labels (a plurality of labels) for the pharmacy staff, wherein the label(s) are used by the pharmacy staff to collect and dispense a plurality of vials 202 for the modular dosage assembly (MAM). Following method step 820, in method step 822, the prescription list entered is compared against a list of vials 202 available to be dispensed. The list of vials available to be dispensed is then selected as per the methods previously described in steps 806, 808, and 810.

System 900 and method 800 can then, optionally choose one or more vials 202 amounts that will provide a final dosage amount within a certain percentage of the prescribed dosage, as discussed above in method step 810. According to still further aspects of the embodiments, system 900 and method 800 can select one more vials 202 based on various criteria including but not limited to a minimum number of vials 202 necessary to provide the final dosage amount, providing a final dosage amount as close as possible to the prescribed dosage amount and combinations thereof.

System 900 and method 800 can then generate an output element consisting of the final dosage amount and/or a list of vials 202 dispensed. The output element can be displayed and/or stored on a patient specific display, patient specific profile, patient specific MAR medication administration record, a prescription (not shown) and combinations thereof.

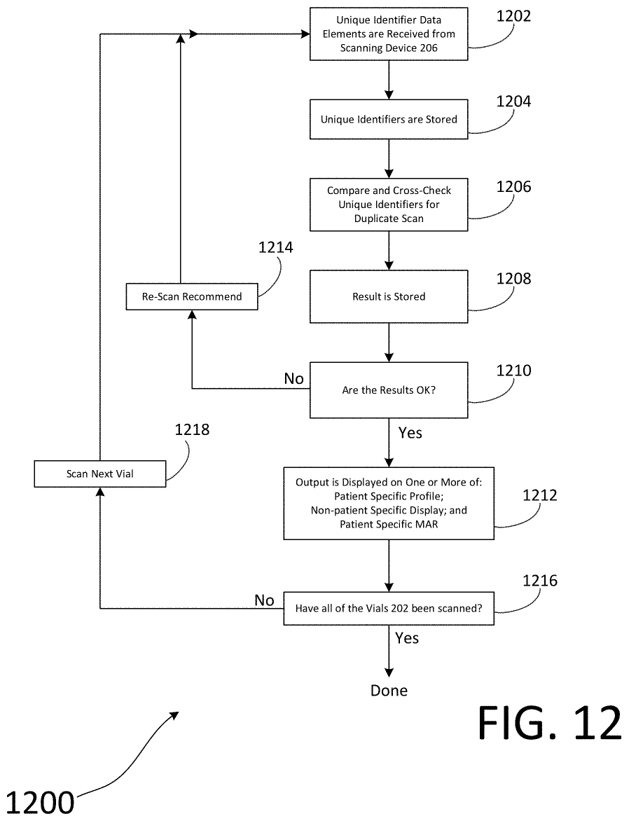

FIG. 12 illustrates a flow chart of method 1200 for comparing data elements obtained from two or more containers comprising part of the modularly assembled medicament container as shown in FIGS. 2A and 2B against each other to eliminate duplicate scanning of the same container/vial 202 (method 1200) according to aspects of the embodiments.

Method 1200 begins with method step 1202 in which an operator scans using scanning device 206 two or more barcodes 204 from at least two different vials 202 that can be attached to, or separate from IV bag 102; in method step 1024, the data associated with each barcode (including unique identifiers), or encoded therein, is retrieved and then stored. As barcode 204 for each new vial 202 is scanned, the information (including/comprising unique identifiers) is compared/cross-checked to those that have already been scanned and stored (step 1206), and the result of the comparison action is also stored (step 1208). The unique identifier information obtained/received and/or stored in any of steps 1202, 1204, and 1206 is not predetermined (meaning the processing and/or storage device implementing method 1200 is not aware of the unique identifier information received prior to processing and/or storing the unique identifier information. In decision step 1210, a determination is made: is the newly scanned barcode 204 data different as one that has already been scanned and/or stored? If the new data (unique identifiers) are the same as ones previously scanned and/or stored, meaning duplicate scans of unique identifiers on barcodes 204 have occurred (“No” path from decision step 1210), then method 1200 proceeds to step 1214 and informs the operator that a re-scan is in order, or some other corrective step needs to occur, and method 1200 proceeds again to method 1200 step 1202.

If, however, the scanned data does not represent a repeat of another, previously scanned vial 202, or other type of duplicate medicament, then method 1200 proceeds to method step 1212 (“Yes” path from decision step 1210). In method step 1212, method 1200 displays the output (e.g. the list of scanned vials 202) on one or more of a patient specific profile, non-patient specific display, and patient specific MAR, according to aspects of the embodiments. Then, following method step 1212, method 1200 proceeds to method step 1216, and questions the operator as to whether all of the vials have been scanned; if all of the vials have been scanned (“Yes” path from decision step 1216), then method 1200 is complete. If, however, the operator indicates that additional vials 202 need to be scanned, then method 1200 proceeds from decisions step 1216 to step 1218 (“No” path from decisions step 1216), in which method 1200 requests that the next vial 202 be scanned, and returns to method step 1202. This process continues until all of the vials 202 have been scanned, its information stored and compared, and verified as being non-redundant, according to aspects of the embodiments.

FIG. 9 illustrates a block diagram of medication administration verification (MAV) system 900 adapted to verify that delivered medicaments match prescribed medicaments according to aspects of the embodiments. Each of the methods described herein can be performed with system 900, as they can be embodied in one or more software programs that can be stored in memory associated with system 900 and/or the one or more processors and/or processing devices as discussed herein.

MAV system 900 comprises one or more scanners 206, one or more MAM containers 200, display 902, computer 904, processor 906, internal data/communications bus (bus) 907, memory 908, program 910, and communications interface(s) 912. In regard to the communications interface 912, each of scanner 206, computer 904, and display 902 has one or more such communications interface 912, although in not all devices are the interfaces 912 the same; that is, in some cases a device's communication interface 912 can be wired and/or wireless, and in some cases a particular device can have one or more of each type. As those of skill in the art can appreciate, such communication devices are well known, and a detailed discussion thereof is both beyond the scope of this discussion, and not needed in order to understand the aspects of the embodiments.

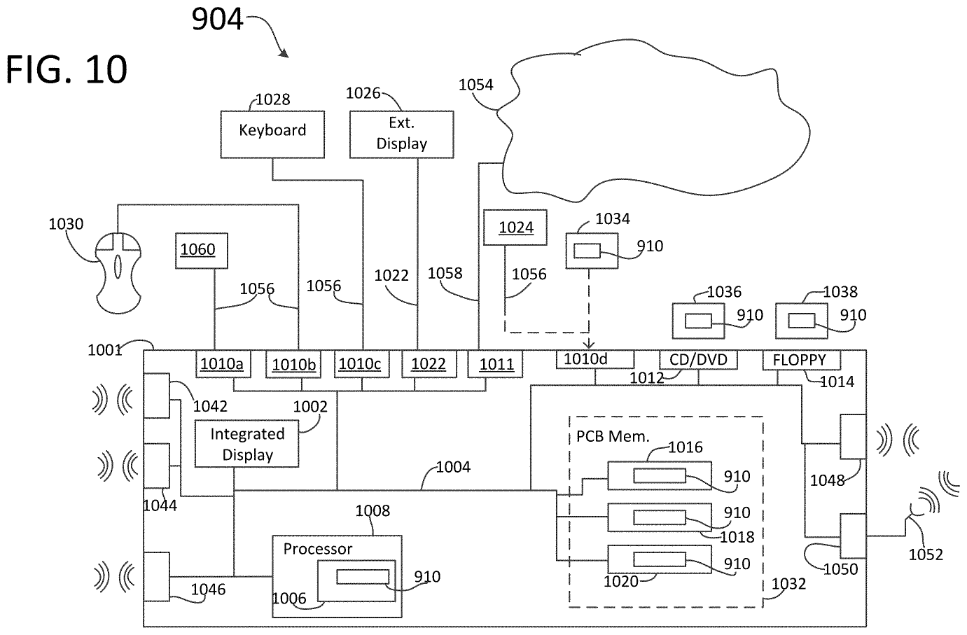

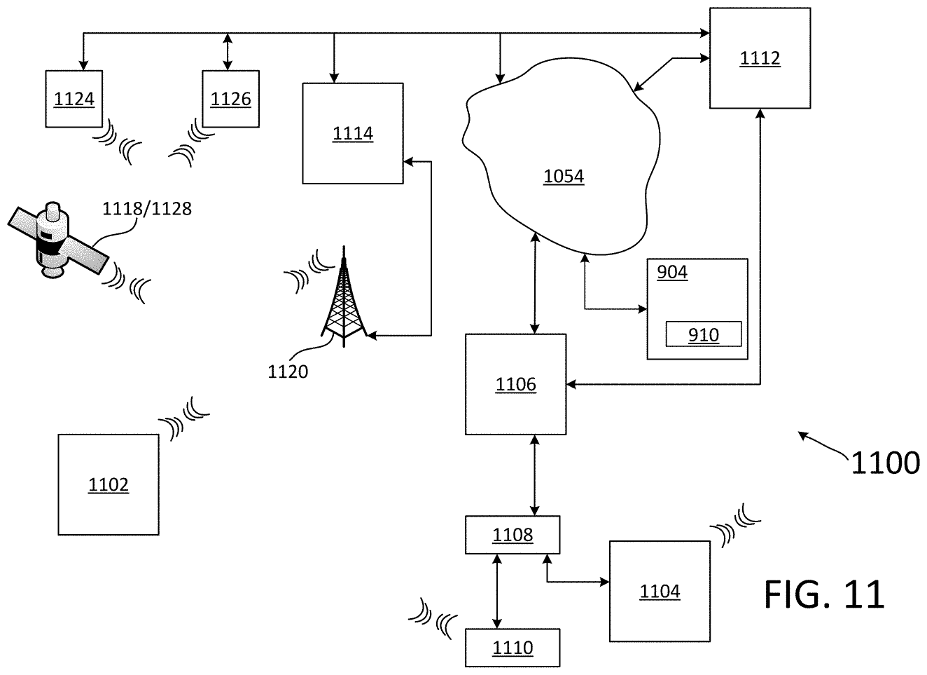

One or more software programs, or applications (Apps) 910 can be stored in memory 908 that is interconnected to processor (may be microprocessor/central processing unit) 906 through bus 907 as those of skill in the art can appreciate. The one or more programs/Apps 910 can be “run” or executed by processor 906 in a manner known to those of skill in the art. The one or more programs/Apps 910 can be executed to perform one or more of the methods as described in detail herein according to aspects of the embodiments. In addition to the one or more programs/Apps 910, there are one or more additional programs or Apps that facilitate communications with scanner 206 and display 902, in a manner known to those of skill in the art. FIGS. 10 and 11, discussed in detail below, illustrate a detailed block diagram of computer 904, and one or more networks within which the aspects of the embodiments can be used within. As such, computer 904 and its operation in one or more networks is discussed in greater detail in regard to FIGS. 10 and 11.

FIG. 10 illustrates a block diagram of components of computer or server 904 suitable for use to implement one or more of the methods 400, 700, and 800 described in FIGS. 4, 7, and 8 according to aspects of the embodiments. MAV computer 904

FIG. 10 illustrates a block diagram of components of computer 904 or server (herein after, “medication administration verification (MAV) computer 904”) suitable for use to implement one or more of the methods 100-600 described in FIGS. 1-6 according to aspects of the embodiments. MAV computer 904 as illustrated in FIG. 10 is suitable for use to implement methods 400, 700, and 800 for various aspects of a medication administration verification system according to aspects of the embodiments. Although MAC computer 904 was briefly discussed in regard to FIG. 9, and several components were described therein, the discussion of FIG. 10 provides additional detail and in some cases similar components have been given new element numbers as shown in FIG. 10. MAV computer 904 includes, among other items, shell/box 1001, internal data/command bus (bus) 1004, processor(s) 1008 (those of ordinary skill in the art can appreciate that in modern server systems, parallel processing is becoming increasingly prevalent, and whereas a single processor would have been used in the past to implement many or at least several functions, it is more common currently to have a single dedicated processor for certain functions (e.g., digital signal processors) and therefore could be several processors, acting in serial and/or parallel, as required by the specific application), universal serial bus (USB) port 1010, compact disk (CD)/digital video disk (DVD) read/write (R/W) drive 1012, floppy diskette drive 1014 (though less used currently, many servers still include this device), and data storage unit 1032. According to further embodiments, a controller can be used in place or, or in conjunction with processor 1008, wherein the controller can include one or more hardware components designed and/or fabricated to replicate the functionality of processor 1008. According to still further embodiments, processor 1008 and a controller can be used interchangeably or in combination to perform the processing functions described herein.

Data storage unit 1032 itself can comprise hard disk drive (HDD) 716 (these can include conventional magnetic storage media, but, as is becoming increasingly more prevalent, can include flash drive-type mass storage devices 1034, among other types), read-only memory (ROM) device(s) 1018 (these can include electrically erasable (EE) programmable ROM (EEPROM) devices, ultra-violet erasable PROM devices (UVPROMs), among other types), and random access memory (RAM) devices 1020. Usable with USB port 1010 is flash drive device 1034, and usable with CD/DVD R/W device 1012 are CD/DVD disks 1036 (which can be both read and write-able). Usable with floppy diskette drive device 1014 are floppy diskettes 1038. Each of the memory storage devices, or the memory storage media (1016, 1018, 1020, 1034, 1036, and 1038, among other types), can contain parts or components, or in its entirety, executable software programming code or application (application, or “App”), referred to as medication administration verification program/application (MAV App) 910, which can implement part or all of the portions of methods 400, 700, and 800 described herein according to aspects of the embodiments. Further, processor 1008 itself can contain one or different types of memory storage devices (most probably, but not in a limiting manner, RAM memory storage media 1020) that can store all or some of the components of MAV App 910.

In addition to the above described components, MAV computer 904 also comprises user console 1024, which can include keyboard 1028, display 1026, and mouse 1030. All of these components are known to those of ordinary skill in the art, and this description includes all known and future variants of these types of devices. Display 1026 can be any type of known display or presentation screen, such as liquid crystal displays (LCDs), light emitting diode displays (LEDs), plasma displays, cathode ray tubes (CRTs), among others. User console 1024 can include one or more user interface mechanisms such as a mouse, keyboard, microphone, touch pad, touch screen, voice-recognition system, among other inter-active inter-communicative devices.

User console 1024, and its components if separately provided, interface with MAV computer 904 via server input/output (I/O) interface 1022, which can be an RS232, Ethernet, USB or other type of communications port, or can include all or some of these, and further includes any other type of communications means, presently known or further developed. MAV computer 904 can further include communications satellite/global positioning system (satellite) transceiver device 1050 to which is electrically connected at least one antenna 1052 (according to an embodiment, there can be at least one GPS receive-only antenna, and at least one separate satellite bi-directional communications antenna). MAV computer 904 can access internet 1054, either through a hard wired connection, via I/O interface 1022 directly, or wirelessly via Wi-Fi transceiver 1042, 3G/4G transceiver 1048 and/or satellite transceiver device 1050 (and their respective antennas) according to an embodiment. MAV computer 904 can also be part of a larger network configuration as in a global area network (GAN) (e.g., internet 1054), which ultimately allows connection to various landlines.

According to further embodiments, user console 1024 provides a means for personnel to enter commands and configuration into MAV computer 904 (e.g., via a keyboard, buttons, switches, touch screen and/or joy stick). Display device 1026 can be used to show visual representations of acquired data, and the status of applications that can be running, among other things.

Bus 1004 provides a data/command pathway for items such as: the transfer and storage of data/commands between processor 1008, Wi-Fi transceiver 1042, BT transceiver 1044, NFC transceiver 1046, internal display 1002, I/O port 1022, USB port 1010, CD/DVD drive 1012, floppy diskette drive 1014, memory 1032, 3G/4G transceiver 1048 and satellite transceiver device 1050. Through bus 1004, data can be accessed that is stored in data storage unit memory 1032. Processor 1008 can send information for visual display to display 1026, and the user can send commands to system operating programs/software/Apps that might reside in either processor 1008.

MAV computer 904, and either memory 1006 or memory 1032, can be used to implement methods 400, 700, and 800 for verifying the administration of the correct types and dosages of medicaments to a patient according to aspects of the embodiments. Hardware, firmware, software or a combination thereof can be used to perform the various steps and operations described herein. According to aspects of the embodiments, MAV App 910, which can carry out the above discussed steps, can be stored and distributed on multi-media storage devices such as devices 1016, 1018, 1020, 1034, 1036 and/or 1038 (described above) or other form of media capable of portably storing information, and storage media 1034, 1036 and/or 1038 can be inserted into, and read by, devices such as USB port 1010, CD-ROM drive 1012, and disk drives 1014, 1016, among other types of software storage devices.

As also will be appreciated by one skilled in the art, the various functional aspects of the embodiments can be embodied in a computer. Accordingly, the embodiments can take the form of an entirely hardware embodiment or an embodiment combining hardware and software aspects. Further, the embodiments can take the form of a non-transitory computer program product stored on a computer-readable storage medium having computer-readable instructions embodied in the medium. Any suitable computer-readable medium can be utilized, including hard disks, CD-ROMs, digital versatile discs (DVDs), optical storage devices, or magnetic storage devices such a floppy disk or magnetic tape. Other non-limiting examples of computer-readable media include flash-type memories or other known types of memories.

Further, those of ordinary skill in the art in the field of the embodiments can appreciate that such functionality can be designed into various types of circuitry, including, but not limited to field programmable gate array structures (FPGAs), application specific integrated circuitry (ASICs), microprocessor based systems, among other types. A detailed discussion of the various types of physical circuit implementations does not substantively aid in an understanding of the embodiments, and as such has been omitted for the dual purposes of brevity and clarity. However, as well known to those of ordinary skill in the art, the systems and methods discussed herein can be implemented as discussed, and can further include programmable devices.

Such programmable devices and/or other types of circuitry as previously discussed can include a processing unit, a system memory, and a system bus that couples various system components including the system memory to the processing unit. The system bus can be any of several types of bus structures including a memory bus or memory controller, a peripheral bus, and a local bus using any of a variety of bus architectures. Furthermore, various types of computer readable media can be used to store programmable instructions. Computer readable media can be any available media that can be accessed by the processing unit. By way of example, and not limitation, computer readable media can comprise computer storage media and communication media. Computer storage media includes volatile and nonvolatile as well as removable and non-removable media implemented in any method or technology for storage of information such as computer readable instructions, data structures, program modules or other data. Computer storage media includes, but is not limited to, RAM, ROM, EEPROM, flash memory or other memory technology, CDROM, digital versatile disks (DVD) or other optical disk storage, magnetic cassettes, magnetic tape, magnetic disk storage or other magnetic storage devices, or any other medium which can be used to store the desired information and which can be accessed by the processing unit. Communication media can embody computer readable instructions, data structures, program modules or other data in a modulated data signal such as a carrier wave or other transport mechanism and can include any suitable information delivery media.

The system memory can include computer storage media in the form of volatile and/or nonvolatile memory such as read only memory (ROM) and/or random access memory (RAM). A basic input/output system (BIOS), containing the basic routines that help to transfer information between elements connected to and between the processor, such as during start-up, can be stored in memory. The memory can also contain data and/or program modules that are immediately accessible to and/or presently being operated on by the processing unit. By way of non-limiting example, the memory can also include an operating system, application programs, other program modules, and program data.

The processor and/or processing device can also include other removable/non-removable, volatile/nonvolatile, and transitory/non-transitory computer storage media. For example, the processor can access a hard disk drive that reads from or writes to non-removable, nonvolatile, and non-transitory magnetic media, a magnetic disk drive that reads from or writes to a removable, nonvolatile, and non-transitory magnetic disk, and/or an optical disk drive that reads from or writes to a removable, nonvolatile, and non-transitory optical disk, such as a CD-ROM or other optical media. Other removable/non-removable, volatile/nonvolatile, and non-transitory computer storage media that can be used in the operating environment include, but are not limited to, magnetic tape cassettes, flash memory cards, digital versatile disks, digital video tape, solid state RAM, solid state ROM and the like. A hard disk drive can be connected to the system bus through a non-removable memory interface such as an interface, and a magnetic disk drive or optical disk drive can be connected to the system bus by a removable memory interface, such as an interface.

The embodiments discussed herein can also be embodied as computer-readable codes on a computer-readable medium. The computer-readable medium can include a computer-readable recording medium and a computer-readable transmission medium. The computer-readable recording medium is any data storage device that can store data which can be thereafter read by a computer system. Examples of the computer-readable recording medium include read-only memory (ROM), random-access memory (RAM), CD-ROMs and generally optical data storage devices, magnetic tapes, flash drives, and floppy disks. The computer-readable recording medium can also be distributed over network coupled computer systems so that the computer-readable code is stored and executed in a distributed fashion. The computer-readable transmission medium can transmit carrier waves or signals (e.g., wired or wireless data transmission through the Internet). Also, functional programs, codes, and code segments to, when implemented in suitable electronic hardware, accomplish or support exercising certain elements of the appended claims can be readily construed by programmers skilled in the art to which the embodiments pertains.

FIG. 11 illustrates network system 1100 within which MAV system 900 can be implemented according to aspects of the embodiments. Much of the network system infrastructure shown in FIG. 11 is or should be known to those of skill in the art, and therefore, in fulfillment of the dual purposes of clarity and brevity, a detailed discussion thereof shall be omitted.

According to aspects of the embodiments, a user of the above described system and method can have MAV App 910 on their mobile device 1102; mobile devices 1102 can include, but are not limited to, so-called smart phones, tablets, personal digital assistants, notebook and laptop computers, and essentially any device that can access the internet and/or cellular phone service or can facilitate transfer of the same type of data in either a wired or wireless manner. For purposes of this discussion, the user shall be discussed as using only mobile device 1102, i.e., a smartphone, though such discussion should be understood to be in a non-limiting manner in view of the discussion above about the other types of devices that can access, use, and provide such information.

In FIG. 11, the user has mobile device 1102, which can access cellular service provider 1114, either through a wireless connection (cellular tower 1120) or via a wireless/wired interconnection (a “Wi-Fi” system that comprises, e.g., modulator/demodulator (modem) 1108, wireless router 1110, personal computer (PC)/server 1104, internet service provider (ISP) 1106, and internet 1054). Further, mobile device 1102 can include near field communications (NFC), “Wi-Fi,” and Bluetooth (BT) communications capabilities as well, all of which are known to those of skill in the art. To that end, network system 1100 further includes, as many homes (and businesses) do, one or more PCs/servers 1104 that can be connected to wireless router 1110 via a wired connection (e.g., modem 1108) or via a wireless connection (e.g., Bluetooth). Modem 1108 can be connected to ISP 1106 to provide internet based communications in the appropriate format to end users (e.g., PC 1104), and which takes signals from the end users and forwards them to ISP 1106. Such communication pathways are well known and understand by those of skill in the art, and a further detailed discussion thereof is therefore unnecessary.

Mobile device 1102 can also access global positioning system (GPS) satellite 1128, which is controlled by GPS station 1124, to obtain positioning information (which can be useful for different aspects of the embodiments), or mobile device 1102 can obtain positioning information via cellular service provider 1114 using cell tower(s) 1120 according to one or more well-known methods of position determination. Some mobile devices 1102 can also access communication satellites 1118 and their respective satellite communication systems control stations 1126 (the satellite in FIG. 11 is shown common to both communications and GPS functions) for near-universal communications capabilities, albeit at a much higher cost than convention “terrestrial” cellular services. Mobile device 1102 can also obtain positioning information when near or internal to a building (or arena/stadium) through the use of one or more of NFC/BT devices, the details of which are known to those of skill in the art. FIG. 11 also illustrates other components of network system 1100 such as plain old telephone service (POTS) provider 1112.

According to further aspects of the embodiments, and as described above, network system 1100 also contains MAV computer 904 that includes MAV App 910, wherein one or more processors, using known and understood technology, such as memory, data and instruction buses, and other electronic devices, can store and implement code that can implement the system and method for verifying administration of the correct medications as prescribed by a physician in a hospital, or other healthcare setting according to aspects of the embodiments. As those of skill in the art can appreciate, in general MAV computer 904 would also use one or more modems 1108, or equivalent devices, in perhaps a distributed fashion, as part of the Cloud, but such devices have been omitted from FIG. 11 in fulfillment of the dual purposes of clarity and brevity.

As described above, one or more encoding processes are discussed in reference to FIGS. 4, 7, and 8. The encoding processes are not meant to limit the aspects of the embodiments, or to suggest that the aspects of the embodiments should be implemented following the encoding processes. The purpose of the encoding processes described herein is to facilitate the understanding of one or more aspects of the embodiments and to provide the reader with one or many possible implementations of the processed discussed herein. FIGS. 4, 7, and 8 illustrate a plurality of flowcharts of various steps performed during the encoding processes. The steps of FIGS. 4, 7, and 8 are not intended to completely describe the encoding process but only to illustrate some of the aspects discussed above.

FIG. 12 illustrates a flow chart/method showing how unique identifiers are used to prevent double scanning of the same barcode 204 of container 202. FIG. 12 may in one or more embodiments, be/represent a computer-implemented method. FIG. 12 may in one or more embodiments, be/represent a software-implemented method. FIG. 12 may in one or more embodiments, be both a computer and software implemented method. FIG. 12 may in one or more embodiments, be implemented on a processing device. FIG. 12 may in one or more embodiments, be implemented on a processing device and a storage device. Step 1202 shows software and/or computer and or processing/storage device receiving unique identifier data elements from scanning device 206. Step 1204 may follow step 1202, in step 1204 the software and/or computer stores the unique identifier information in/on a memory and/or a storage element. After step 1204 the method may proceed to step 1206 (optionally, step 1202 may proceed directly to step 1206 bypassing step 1204), wherein in step 1206 the software and/or computer compares/cross-checks/looks for a match of at least two (scanned) unique identifiers obtained from barcodes 204a-204d in FIG. 2 (and optionally or additionally barcode 104). The software and/or computer may then proceed to step 1208 where the result of the unique identifier comparison/cross-check/match is stored on a computer and/or software and/or server memory and/or storage element and/or memory element. Step 1208 may then proceed to step 1210 (optionally step 1206 may bypass step 1208 and may proceed directly step 1210). In step 1210 the software and/or computer may issue an output of “OK to proceed” or “NOT OK to proceed” with administration of the modular medicament assembly. The output may be displayed 1212, 1214 on a patient specific screen such as a patient profile, a patient medication profile, a patient medication list, a medication administration record MAR, a patient specific medication administration record MAR, a patient specific prescription, and combinations thereof. Optionally, alternately, or additionally, the output may be displayed on a non-patient specific display. If the output is “OK to proceed” with administration of the modular medicament assembly a positive output may be displayed in the form of a check mark. The check mark may be green in color. An audible sound may also be associated and/or produced with the positive output. If the output is negative, meaning “NOT OK to proceed with administration of the modular medicament assembly and “X” may be displayed. The “X” may be red in color and may also be associated with an audible sound. If the output is negative a “RESCAN” 1214 warning may be displayed on a patient specific and/or on a non-patient specific screen/display and the method goes back to step 1202 where the entire method needs to be repeated. Other alerts using different symbols and colors are also contemplated that may alert a user if it is ok or not ok to proceed with administration of the modularly assembled medicament.

The unique identifiers provided by the invention are unique and are intended to provide an extra layer of safety for modularly assembled medicaments (and/or a plurality of containers for a modular medicament assembly). The unique identifiers may be embedded in and/or on barcodes of containers for easy of use, so a scanner may easily scan the barcodes to acquire the unique identifier information stored in the barcodes. Unique identifiers in other embodiments may be visually displayed on containers so a user would need to manually type in the unique identifier located on each container into a keyboard and computer/software.

The unique identifiers disclosed and discussed in the herein specification may be used with any of the systems, devices, methods, containers discussed/disclosed and/or in any combinations. The unique identifiers and methods of disclosed pertaining to the unique identifiers may be used for computer implemented methods, software implemented methods, computer and software implemented methods, processing devices, and combinations thereof. Applications (Apps), handheld devices, smartphones, and the similar devices and software may by used in systems that will accommodate the use of unique identifiers for determining if a container of a beneficial substance has been duplicately (double) scanned.

The unique identifiers disclosed and/or discussed in the herein specification may be applied, in one or more embodiments, to oral dosage forms and/or to medicaments not intended for a modular medicament assembly. In one non-limiting example, a patient is prescribed 75 milligrams (mg) of a Benadryl to be given orally. The pharmacy dispenses three tablets, each tablet containing Benadryl 25 mg for a final dosage amount of 75 mg. Each tablet of Benadryl is packaged in a blister pack (although any type of packaging may be used) with a barcode provided for each tablet. Each barcode has a unique identifier. Therefore, when a nurse or other healthcare provider scans with a scanner each of the three barcodes associated with each of the three Benadryl 25 mg tablets a scanner (acquisition device) acquires three separate unique identifiers for each of the Benadryl 25 mg tables (even though each tablet is the same drug at the same and/or at about the same dose of 25 mg). The computer and/or software implemented method discussed in FIG. 12 will then compare each of the unique identifiers against each other, will determine that each unique identifier is different, and will subsequently issue an output that it is “OK to proceed” with administration of the drug because there were no double scans and the final dosage amount is the same as the prescribed dosage amount of 75 mg. This is just one non-limiting example of how unique identifiers for medicaments may be used in non-modularly assembled scenarios such as for oral dosage forms such as tablets, capsules, pills, and the such.

In one or more embodiments, method 700 may be implemented concurrently/simultaneously with the method of FIG. 12. In accordance with this embodiment, a user scans each barcode 204a-204d of vials 202a-202d and optionally scans barcode 104 of container 102 with acquisition device (scanner) 206. The barcodes 204a-204d contain the identity (name) and quantity of drug in each of vials 202a-202d and also a unique identifier (code) for each of vials 202a-202d. This is shown in FIG. 7 element 706. Next step 708 (of FIG. 7) shows that doses for all the vials 202a-202d are summed (added) and/or a list of all medicaments in containers 202a-202d and doses is created, additionally a list of all of the unique identifiers obtained by the scanning device 206 from barcodes 204a-204d is also created. In FIG. 7, the next step in the method is any one, optionally and/or alternately any combination, of Method 1 (step 710), Method 2 (step 712), Method 3 (step 714), and/or Method 4 (step 716). Any of Methods 1-4 may additionally comprise (further comprise) a software and/or a computer implemented method of comparing and/or cross-checking and/or matching the unique identifiers obtained from barcodes 204a-204d to determine if a double (duplicate) scan of any of vials 202a-202d occurred. The method of comparing/cross-checking/matching the unique identifiers may occur prior to, at about the time, at the time, and/or after any of step 710, 712, 714, and/or 716 of method 700. FIG. 7 step 718 shows an alert being provided if it is OK or NOT OK to proceed with administration of the modular medicament assembly. The Alert (alert is an output) may comprise two alerts, a first alert informing the user if the final dosage amount provided is the same as the prescribed dosage amount and a second alert informing the user if a duplicate scan (double scan of any of vials 202a-202d) has occurred. These two alerts may be displayed simultaneously, at about the same time, and/or sequentially. Optionally, or alternately, if either (or both) of the alerts is negative (if either, or both, the final dosage amount provided is not the same as the prescribed amount or a duplicate scan has occurred) then a negative output of NOT OK to proceed with administration of drug will be provided. Optionally, or alternately, if both alerts are positive (both the final dosage amount provided is the same as the prescribed amount and no duplicate scans have occurred) then a positive output of OK to proceed with administration of drug will be provided. Therefore, as has been discussed in detail in this paragraph, FIG. 7 method 700 is operable with the method of FIG. 12, in one or more embodiments of the invention. In one or more embodiments, at least one of method 700 and method of FIG. 12 may be a computer implemented method, a software implemented method, and combinations thereof.

The disclosed embodiments provide a source array, computer software, and a method for verifying administration of the correct medications as prescribed by a physician in a hospital, or other healthcare setting according to aspects of the embodiments. It should be understood that this description is not intended to limit the embodiments. On the contrary, the embodiments are intended to cover alternatives, modifications, and equivalents, which are included in the spirit and scope of the embodiments. Further, in the detailed description of the embodiments, numerous specific details are set forth to provide a comprehensive understanding of the claimed embodiments. However, one skilled in the art would understand that various embodiments can be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter can include other examples that occur to those skilled in the art.

The above-described embodiments are intended to be illustrative in all respects, rather than restrictive, of the embodiments. Thus the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

1. A method for verifying contents of modularly assembled medicament containers, comprising:
providing at least two containers for a modularly assembled medicament, the at least two containers containing a medicament, each container having a data-containing tag, the data-containing tag comprising information pertaining to an amount and type of the medicament in the container;
scanning data-containing tags on at least two of the medicament containers; and
verifying that the type and amount of the medicaments in the at least two containers matches the prescribed type and amount of the medicament.

2. The method of claim 1, wherein the step of verifying comprises:
obtaining prescription information from at least one of a memory element and a storage element.
obtaining information from the data-containing tag of the medicament container based on the scanning step of the data-containing tag of the medicament container; and
matching the information from the medicament container data-containing tag with prescription information.

3. The method of claim 1, wherein the method is a computer implemented method.

4. The method of claim 2, wherein the method is a computer implemented method.

5. The method of claim 1, wherein the prescribed type and amount of the medicament is for a specific patient.

6. The method of claim 1, wherein the prescribed type and amount of the medicament is linked to a patient specific identifier.

7. The method of claim 2, wherein the prescribed type and amount of the medicament is for a specific patient.

8. The method of claim 2, wherein the prescribed type and amount of the medicament is linked to a patient specific identifier.

9. The method of claim 2, wherein the step of matching comprises:
summing dosage amount information of the at least two medicament containers (summed dosage of vial container medicaments);
obtaining a final dosage amount information from prescription list of medicaments; and
determining that the summed dosage amount of the at least two medicament containers is equal or not equal to the final prescribed dosage amount.

10. The method of claim 2, wherein the step of matching comprises:
summing dosage amount information of the at least two medicament containers (summed dosage of vial container medicaments); and
summing dosage amount information of the at least two medicaments listed in a patient specific prescription list (summed dosage of prescription list medicaments);
determining that the summed dosage of prescription list medicaments is equal to the summed dosage of vial container medicaments.

11. The method of claim 2, wherein the step of matching comprises:
obtaining a list of the dosage amounts from the at least two medicament containers (list of dosages of vial container medicaments);
obtaining a list of the dosage amount information of the at least two medicaments listed in a patient specific profile (list of dosage amounts from prescription list of medicaments); and
determining that the list of dosages of from the prescription list of medicaments matches the list of dosages of vial container medicaments.

12. The method of claim 2, wherein the step of matching comprises:
obtaining a list of dosage amount information of the at least two medicament containers (list of dosages of vial container medicaments);
obtaining a final total dosage amount of the at least two medicaments listed in a patient specific profile (total final dosage amount); and
comparing the list of dosages of the vial container medicaments to the total final dosage amount.

13. The method of claim 2, wherein the step of matching comprises:
obtaining a list of dosage amount information of the at least two medicament containers (list of dosages of vial container medicaments);
summing dosage amount information of the at least two medicaments listed in a patient specific profile (summed dosage of prescription list medicaments); and
determining that the summed dosage of prescription list medicaments is equal to the list of dosages of vial container medicaments.

14. The method of claim 1, further comprising issuing a first alert if a no match condition exists.

15. The method of claim 1, further comprising issuing a second alert if a match condition exists.

16. The method of claim 1, wherein the method is a computer implemented method.

17. The method of claim 1, the data-containing tag further comprising a unique identifier.

18. The method of claim 2, the data-containing tag further comprising a unique identifier, the unique identifier characterized in that it differentiates any two containers from each other thus preventing duplicate (double) scanning of the same container.

19. The method of claim 2, the method further comprising generating an output, the output characterized in that it alerts a user if a duplicate scan has occurred or if a duplicate scan has not occurred.
